(19) United States
(12) Patent Application Publication
(10) Pub. No.: US 2024/0299450 A1
(43) Pub. Date: Sep. 12, 2024

(54) USES OF AMPHIPHILES IN IMMUNE CELL THERAPY AND COMPOSITIONS THEREFOR

(71) Applicant: Elicio Therapeutics, Inc., Boston, MA (US)

(72) Inventors: Dylan DRAKES, Boston, MA (US); Peter C. DEMUTH, Boston, MA (US)

(21) Appl. No.: 18/281,112

(22) PCT Filed: Mar. 10, 2022
(86) PCT No.: PCT/US2022/019723

§ 371 Date: Sep. 8, 2023

Related U.S. Application Data
(60) Provisional application No. 63/306,247, filed on Feb. 3, 2022.
(60) Provisional application No. 63/286,854, filed on Dec. 7, 2021.
(60) Provisional application No. 63/255,829, filed on Oct. 14, 2021.
(60) Provisional application No. 63/159,237, filed on Mar. 10, 2021.

(51) Int. Cl. A61K 35/17; A61K 39/00; A61K 39/39; A61K 47/54; A61P 35/00; A61P 37/04

(52) U.S. Cl. CPC A61K 35/17; A61K 39/39; A61K 39/4611; A61K 39/4632; A61K 39/464464; A61K 39/464488; A61K 39/464838; A61K 47/544; A61P 35/00; A61P 37/04

(57) ABSTRACT

The disclosure features amphiphilic ligand conjugates including a peptide or a ligand for a mucosal-associated invariant T-cell and a lipid and T cell receptor modified immune cells. The disclosure also features compositions and methods of using the same, for example, to stimulate proliferation of T cell receptor expressing cells.

### CROSS-REFERENCE TO RELATED APPLICATIONS

The present application claims benefit of the filing dates of U.S. Provisional Application No. 63/159,237, filed Mar. 10, 2021, U.S. Provisional Application No. 63/255,829, filed Oct. 14, 2021, U.S. Provisional Application No. 63/286,854, filed Dec. 7, 2021, and U.S. Provisional Application No. 63/306,247, filed Feb. 3, 2022, each of which is hereby incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 2, 2022, is named 51026-040WO5_Sequence_Listing_3_1_22_ST25 and is 980,231 bytes in size.

### BACKGROUND OF THE INVENTION

Cancer is one of the leading causes of death in the world, with over 14 million new cancer cases diagnosed and over eight million cancer deaths occurring each year. The American Cancer Society estimates 1,762,450 new cases of cancer and 606,880 cancer deaths in the United States in 2019. While several treatments for cancer have been developed, the disease still remains a significant problem.

There thus exists a need for improved treatments for cancer.

### SUMMARY OF THE INVENTION

The invention provides, inter alia, use of amphiphilic ligand conjugates including a lipid, a peptide, and optionally a linker, in combination with a T cell receptor (TCR) modified immune cell for stimulating an immune response in a subject.

In one aspect, the invention provides a method of stimulating an immune response to a target cell population or target tissue in a subject including administering to the subject (1) an amphiphilic ligand conjugate including a lipid, a peptide, and, optionally, a linker, and (2) a T-cell receptor (TCR) modified immune cell, wherein the TCR binds the peptide of the amphiphilic ligand conjugate.

In another aspect, the invention provides use of (1) an amphiphilic ligand conjugate including a lipid, a peptide, and, optionally, a linker, and (2) a T-cell receptor (TCR) modified immune cell, wherein the TCR binds the peptide of the amphiphilic ligand conjugate, for stimulating an immune response to a target cell population or target tissue in a subject.

In another aspect, the invention provides use of (1) an amphiphilic ligand conjugate including a lipid, a peptide, and, optionally, a linker, and (2) a T-cell receptor (TCR) modified immune cell, wherein the TCR binds the peptide of the amphiphilic ligand conjugate, for the manufacture of a medicament for stimulating an immune response to a target cell population or target tissue in a subject.

In another aspect, the invention provides (1) an amphiphilic ligand conjugate including a lipid, a peptide, and, optionally, a linker, and (2) a T-cell receptor (TCR) modified immune cell, wherein the TCR binds the peptide of the amphiphilic ligand conjugate, for use in stimulating an immune response to a target cell population or target tissue in a subject.

In another aspect, the invention provides a method of stimulating an immune response to a target cell population or target tissue in a subject including administering to the subject (1) an amphiphilic ligand conjugate comprising a lipid, a ligand for a mucosal-associated invariant T-cell (MAIT), and, optionally, a linker, and (2) a T-cell receptor (TCR) modified immune cell, wherein the TCR binds the ligand of the amphiphilic ligand conjugate.

In another aspect, the invention provides use of (1) an amphiphilic ligand conjugate including a lipid, a peptide, and, optionally, a linker, and (2) a T-cell receptor (TCR) modified immune cell, wherein the TCR binds the peptide of the amphiphilic ligand conjugate, for stimulating an immune response to a target cell population or target tissue in a subject.

In another aspect, the invention provides use of (1) an amphiphilic ligand conjugate including a lipid, a ligand for a MAIT cell, and, optionally, a linker, and (2) a T-cell receptor (TCR) modified immune cell, wherein the TCR binds the ligand of the amphiphilic ligand conjugate, for the manufacture of a medicament for stimulating an immune response to a target cell population or target tissue in a subject.

In another aspect, the invention provides (1) an amphiphilic ligand conjugate including a lipid, a ligand for a MAIT cell, and, optionally, a linker, and (2) a T-cell receptor (TCR) modified immune cell, wherein the TCR binds the ligand of the amphiphilic ligand conjugate, for use in stimulating an immune response to a target cell population or target tissue in a subject.

In another aspect, the invention provides a method of stimulating an immune response to a target cell population or target tissue in a subject including administering to the subject an amphiphilic ligand conjugate including a lipid, a ligand for a MAIT cell, and, optionally, a linker.

In another aspect, the invention provides use of an amphiphilic ligand conjugate including a lipid, a ligand for a MAIT cell, and, optionally, a linker, for stimulating an immune response to a target cell population or target tissue in a subject.

In another aspect, the invention provides use of an amphiphilic ligand conjugate including a lipid, a ligand for a MAIT cell, and, optionally, a linker, and for the manufacture of a medicament for stimulating an immune response to a target cell population or target tissue in a subject.

In some embodiments, the method further includes administering an adjuvant to the subject. In some embodiments, the adjuvant is an amphiphilic oligonucleotide conjugate including an immunostimulatory oligonucleotide conjugated to a lipid, with or without a linker.

In some embodiments, the lipid of the amphiphilic ligand conjugate inserts into a cell membrane under physiological conditions, binds albumin under physiological conditions, or both. In some embodiments, the lipid of the amphiphilic ligand conjugate is a diacyl lipid. In some embodiments, the diacyl lipid of the amphiphilic ligand conjugate includes acyl chains including 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29 or 30 hydrocarbon units. In some embodiments, the lipid is 1,2-distearoyl-sn-glycero-3-phosphoethanolamine (DSPE). In some embodiments, the linker is selected from the group consisting of a hydrophilic polymer, a string of hydrophilic amino acids, a polysaccharide, and an oligonucleotide, or a combination thereof. In some embodiments, the linker includes “N” polyethylene glycol units, wherein N is between 24-50 (e.g., 24-30, 30-35, 35-40, 40-45, 45-50, 24-40, 35-50, or 30-40). In some embodiments, the linker includes PEG24-amido-PEG24. In some embodiments, the “N” polyethylene glycol units are consecutive.

In some embodiments, the peptide is an antigen, or a fragment thereof. In some embodiments, the fragment is an immunogenic fragment. In some embodiments, the antigen, or fragment thereof, is a tumor-associated antigen. In some embodiments, the antigen, or fragment thereof, includes between 3 amino acids and 50 amino acids (e.g., 3-10 amino acids, 10-20 amino acids, 20-30 amino acids, 30-40 amino acids, 40-50 amino acids, 5-20 amino acids, 30-50 amino acids, or 20-40 amino acids). In some embodiments, the antigen includes any one of Human papillomavirus (HPV) E6 protein (e.g., HPV-6 E6 protein (SEQ ID NO: 1172), HPV-11 E6 protein (SEQ ID NO: 1171), HPV-16 E6 protein (SEQ ID NO: 1169), or an HPV-18 E6 protein (SEQ ID NO: 1170)), HPV E7 protein (e.g., HPV-6 E7 protein (SEQ ID NO: 1173), HPV-11 E7 protein (SEQ ID NO: 1174), HPV-16 E7 protein (SEQ ID NO: 1175), or an HPV-18 E7 protein (SEQ ID NO: 1176)), Kirsten rat sarcoma (mKRAS) (SEQ ID NO: 1177) (e.g., G12A, G12C, G12D, G12E, G12F, G12H, G121, G12K, G12L, G12M, G12N, G12P, G12Q, G12R, G12S, G12T, G12V, G12W, G12Y, G13C, G13D, Q61A, Q61C, Q61 E, Q61F, Q61G, Q61H, Q611, Q61K, Q61L, Q61M, Q61N, Q61N, Q61P, Q61R, Q61T, Q61V, and Q61W variants), Wilms tumor 1 (WT-1) (SEQ ID NO: 19), New York Esophageal Squamous Cell Carcinoma (NYESO) (SEQ ID NO: 9), Mucin 1 (MUC1) (SEQ ID NO: 67), Epidermal growth factor receptor (EGFR) (SEQ ID NO: 1125), Epidermal growth factor receptor variant III (EGFRviii), Phosphoinositide 3-kinase (PI3K) (SEQ ID NO: 1126), Latent membrane protein 2 (LMP2) (SEQ ID NO: 17), Receptor tyrosine-protein kinase erbB-2 (HER-2/neu) SEQ ID NO: 70), Melanoma antigen A3 (MAGE A3) (SEQ ID NO: 12), p53 wild-type (SEQ ID NOS: 86 and 89), p53 mutant, Prostate-specific membrane antigen (PSMA) (SEQ ID NO: 1127), Ganglioside G2 (GD2), Ganglioside G3 (GD3), Carcinoembryonic antigen (CEA) (SEQ ID NO: 1128), Melanoma antigen recognized by T cells (MelanA/MART-1) (SEQ ID NO: 8), Glycoprotein 100 (gp100) (SEQ ID NO: 1129), Proteinase3 (SEQ ID NO: 1130), Breakpoint cluster region protein-Tyrosine protein kinase (bcr-abl) (SEQ ID NO: 1131), Tyrosinase (SEQ ID NOS: 11 and 25), Survivin (SEQ ID NO: 1132), Prostate-specific antigen (PSA) (SEQ ID NO: 1133), human Telomerase reverse transcriptase (hTERT) (SEQ ID NO: 1134), Ephrin type-A receptor 2 (EphA2) (SEQ ID NO: 1135), Pancreatitis associated protein (PAP) (SEQ ID NO:1136), Mucolipidaryl hydrocarbon receptor-interacting protein (ML-AIP) (SEQ ID NO: 1178), Alpha fetoprotein (AFP) (SEQ ID NO: 1137), Epithelial cell adhesion molecule (EpCAM) (SEQ ID NO: 1138), ETS-related gene (ERG) (SEQ ID NO: 1139) (e.g., TMOPRSS2 ETS fusion), NA17 (SEQ ID NO: 1140), Paired Box 3 (PAX3) (SEQ ID NO: 1141), Anaplastic lymphoma kinase (ALK) (SEQ ID NO: 1142), androgen receptor (SEQ ID NO: 1143), Cyclin B (SEQ ID NO: 1144), N-myc proto-oncogene protein (MYCN) (SEQ ID NO: 1145), Rho protein coding (RhoC) (SEQ ID NO: 1146), Tyrosinase-related protein-2 (TRP-2) (SEQ ID NO: 1147), Mesothelin (SEQ ID NO: 1148), Prostate stem cell antigen (PSCA) (SEQ ID NO:1149), Melanoma antigen A1 (MAGE A1) (SEQ ID NO: 15), Cytochrome P450 Family 1 Subfamily B Member 1 (CYP1 B1) (SEQ ID NO: 1150), Placenta-specific protein 1 precursor (PLAC1) (SEQ ID NO: 1151), Monosialodihexosylganglioside (GM3), Brother of regulator of imprinted sites (BORIS) (SEQ ID NO: 1152), Tenascin (Tn) (SEQ ID NO: 1153), Globohexasylceraminde (GloboH), Translocation-Ets-leukemia virus protein-6—acute myeloid leukemia 1 protein (ETV6-AML) (SEQ ID NO: 1154), NY breast cancer antigen 1 (NY-BR-1) (SEQ ID NO: 1179), Regulator of G protein signaling 5 (RGS5) (SEQ ID NO: 1155), Squamous cell carcinoma antigen recognized by T cells 3 (SART3) (SEQ ID NOS: 1156), Salmonella enterotoxin (STn) (SEQ ID NO: 1157), Carbonic Anhydrase IX (SEQ ID NO: 42), Paired box gene 5 (PAXS) (SEQ ID NO: 1158), Cancer testis antigen (OY-TES1) (SEQ ID NO: 1159), Tyrosine-protein kinase Lck (LCK) (SEQ ID NO: 1160), human high molecular weight-melanoma associated antigen (HMWMAA) (SEQ ID NO: 1180), A-kinase anchoring protein 4 (AKAP-4) (SEQ ID NO: 1161), Protein SSX2 (SSX2) (SEQ ID NO: 88), X-antigen family member 1 (XAGE-1) (SEQ ID NO: 1162), B7 homolog 3 (B7H3) (SEQ ID NO: 1163), Legumain (SEQ ID NO: 1164), Tyrosine-protein kinase receptor (Tie 2) (SEQ ID NO: 1165), P antigen family member 4 (Page4) (SEQ ID NO: 1166), Vascular endothelial growth factor receptor 2 (VEGFR2) (SEQ ID NO: 1167), Melanoma-cancer testis antigen 1 (MAD-CT-1) (SEQ ID NO: 1182), Fibroblast activation protein (FAP) (SEQ ID NO: 1181), Platelet derived growth factor receptor beta (PDGFR-B) (SEQ ID NO: 1168), Melanoma-cancer testis antigen 2 (MAD-CT-2) (SEQ ID NO: 1183). In some embodiments, the antigen includes a fragment of the sequence of any one of SEQ ID NOs: 1-97 or 1125-1183, or includes Ganglioside G2 or Ganglioside G3. In some embodiments, the peptide includes an amino acid sequence of any one of SEQ ID NOs: 98-1123. In some embodiments, the ligand for a MAIT cell is a small molecule metabolite ligand. In some embodiments, the ligand for a MAIT cell is a valine-citrulline-p-aminobenzyl carbamate modified ligand. In some embodiments, the valine-citrulline-p-aminobenzyl carbamate modified ligand is a valine-citrulline-p-aminobenzyl carbamate modified 5-amino-6-D-ribityl prodrug. In some embodiments, the ligand for a MAIT cell is a riboflavin metabolite or a drug metabolite. In some embodiments, the riboflavin metabolite is 5-(2-oxopropylideneamino)-6-d-ribitylaminouracil, 5-(2-oxoethylideneamino)-6-D-ribitylaminouracil, 6,7-dimethyl-8-D-ribityllumazine, 7-hydroxy-6-methyl-8-D-ribityllumazine, 6-hydroxymethyl-8-D-ribityl-lumazine, 6-(1H-indol-3-yl)-7-hydroxy-8-ribityllumazine, or 6-(2-carboxyethyl)-7-hydroxy8-ribityllumazine. In some embodiments, the drug metabolite is benzbromarone, chloroxine, diclofenac, 5-hydroxy diclofenac, 4-hydroxy diclofenac, floxuridine, galangin, menadione sodium bisulfate, mercaptopurine, or tetrahydroxy-1,4-quinone hydrate.

In some embodiments, the amphiphilic ligand conjugate is trafficked to a lymph node. In some embodiments, the amphiphilic ligand conjugate is trafficked to an inguinal lymph node or an axillary lymph node. In some embodiments, the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

In some embodiments, the immune cell is a T cell, a B cell, a natural killer (NK) cell, a macrophage, a neutrophil, a dendritic cell, a mast cell, an eosinophil, or a basophil. In some embodiments, the immune cell is a T cell. In some embodiments, the immune cell is a human mucosal-associated T (MAIT) cell.

In some embodiments, the immune response is an anti-tumor immune response. In some embodiments, the target cell population or the target tissue is a tumor cell population or a tumor tissue. In some embodiments, the method includes reducing or decreasing the size of the tumor tissue or inhibiting growth of the tumor cell population or the tumor tissue in the subject. In some embodiments, the method includes activating the immune cell, expanding the immune cell, and/or increasing proliferation of the immune cell. In some embodiments, activating the immune cell, expanding the immune cell, and/or increasing proliferation of the immune cell is performed ex vivo. In some embodiments, activating the immune cell, expanding the immune cell, and/or increasing proliferation of the immune cell is performed in vivo.

In some embodiments, the subject has a disease, a disorder, or a condition associated with expression or elevated expression of the antigen. In some embodiments, the subject is lymphodepleted prior to the administration of the amphiphilic ligand conjugate and TCR modified immune cell. In some embodiments, the lymphodepletion is by sublethal irradiation. In some embodiments, the subject is administered the amphiphilic ligand conjugate prior to receiving the immune cell including the TCR. In some embodiments, the subject is administered the amphiphilic ligand conjugate after receiving the immune cell including the TCR. In some embodiments, the amphiphilic ligand conjugate and the immune cell including the TCR are administered simultaneously.

In some embodiments, the amphiphilic ligand conjugate and/or the TCR modified immune cell are administered in a composition including a pharmaceutically acceptable carrier. In some embodiments, the composition further includes an adjuvant. In some embodiments, the adjuvant is an amphiphilic oligonucleotide conjugate including an immunostimulatory oligonucleotide conjugated to a lipid, with or without a linker.

In another aspect, the invention provides a method of activating, proliferating, phenotypically maturing, or inducing acquisition of cytotoxic function of a TCR modified T-cell in vitro, including culturing the TCR modified T-cell in the presence of a dendritic cell including an amphiphilic ligand conjugate including a lipid, a peptide, and, optionally, a linker.

In a further aspect, the invention provides an amphiphilic ligand conjugate including a lipid, a peptide, and, optionally, a linker, where peptide includes an amino acid sequence of any one of SEQ ID NOs: 98-1123.

In another aspect, the invention provides an amphiphilic ligand conjugate including a lipid, a peptide, and, optionally, a linker, where peptide includes a fragment of the sequence of any one of SEQ ID NOs: 1-97 or 1125-1183, or includes Ganglioside G2 or Ganglioside G3.

In another aspect, the invention provides a method of activating, proliferating, phenotypically maturing, or inducing acquisition of cytotoxic function of a TCR modified T-cell in vitro, including culturing the TCR modified T-cell in the presence of a dendritic cell including an amphiphilic ligand conjugate including a lipid, a ligand for a MAIT cell, and, optionally, a linker.

### BRIEF DESCRIPTION OF THE DRAWINGS

FIG. 1A-FIG. 1B are graphs showing the number of pmel T cells in the peripheral blood of mice, that were administered 5×10⁵ B16F110 melanoma tumor cells on day −7, 5 days (FIG. 1A) and 19 days (FIG. 11B) after being administered PBS (leftmost, circles), soluble (sol) gp100 (middle, circles), amphiphilic (amph) gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 5 mice (n=5).

FIG. 2A is a graph showing the percentage of mouse survival over time after being injected with 5×10⁵ B16F110 melanoma tumor cells on day −7 for mice who were administered PBS (middle, dotted line), soluble gp100 (leftmost, dotted line), amphiphilic gp100 (rightmost, dotted line), 1×10⁶ pmel T cells (leftmost, dashed line), 1×10⁶ pmel T cells and soluble gp100 (middle dashed line), 1×10⁶ pmel T cells and amphiphilic gp100 (rightmost, dashed line), 5×10⁶ pmel T cells (leftmost, solid line), 5×10⁶ pmel T cells and soluble gp100 (middle, solid line), or 5×10⁶ pmel T cells and amphiphilic gp100 (rightmost, solid line), where 10 mice were observed for each group (n=10).

FIG. 2B is a graph showing the tumor volume in mice over time after being injected with 5×10⁵ B16F110 melanoma tumor cells on day −7 for mice who were administered PBS (middle, dotted line and circles), soluble gp100 (leftmost, dotted line and circles), amphiphilic gp100 (rightmost, dotted line and circles), 1×10⁶ pmel T cells (leftmost, dashed line and squares), 1×10⁶ pmel T cells and soluble gp100 (middle, dashed line and squares), 1×10⁶ pmel T cells and amphiphilic gp100 (rightmost, dashed line and squares), 5×10⁶ pmel T cells (leftmost, solid line and triangles), 5×10⁶ pmel T cells and soluble gp100 (middle, solid line and triangles), or 5×10⁶ pmel T cells and amphiphilic gp100 (rightmost, solid line and triangles), where 5 or 10 mice were observed for each group (n=5 or 10).

FIG. 3A-FIG. 3D are graphs showing the number of pmel T cells in the peripheral blood of mice which had previously rejected a tumor following adoptive T cell transfer and administration of 1×10⁶ pmel T cells and 10 μg of amphiphilic gp100 (squares) or 5×10⁶ pmel T cells and 10 μg of amphiphilic gp100 vaccine (triangles) in comparison to a tumor naïve control group (circles) that were challenged with a second 5×10⁵ dose of B16F10 melanoma tumor cells on day 75 post initial adoptive T cell transfer after 0 days (FIG. 3A), 7 days (FIG. 3B), 14 days (FIG. 3C), and 21 days (FIG. 3D), where 4 or 7 mice were observed for each group (n=4 or 7).

FIG. 4A is a graph showing the survival rate of mice over time that had previously rejected a tumor following adoptive T cell transfer and administration of 1×10⁶ pmel T cells and 10 μg of amphiphilic gp100 (dashed line) or 5×10⁶ pmel T cells and 10 μg of amphiphilic gp100 vaccine (solid line staying at 100% survival) in comparison to a tumor naïve control group (solid line dropping towards 0% survival past 20 days) that were challenged with a second 5×10⁵ dose of B116F110 melanoma tumor cells on day 75 post initial adoptive T cell transfer, where 4 or 7 mice were observed for each group (n=4 or 7).

FIG. 4B is a graph showing the tumor volume in mice over time that had previously rejected a tumor following adoptive T cell transfer and administration of 1×10⁶ pmel T cells and 10 μg of amphiphilic gp100 (dashed line) or 5×10⁶ pmel T cells and 10 μg of amphiphilic gp100 vaccine (flat solid line at 0) in comparison to a tumor naïve control group (solid lines, left of dashed line) that were challenged with a second 5×10⁵ dose of B16F10 melanoma tumor cells on day 75 post initial adoptive T cell transfer, where 4 or 7 mice were observed for each group (n=4 or 7).

FIG. 5A and FIG. 5B are graphs showing the amount of pmel T cells in peripheral blood from mice that previously rejected a tumor following adoptive T cell transfer and administration of 1×10⁶ pmel T cells and 10 μg of amphiphilic gp100 (squares) or 5×10⁶ pmel T cells and 10 μg of amphiphilic gp100 vaccine (triangles) in comparison to a tumor naïve control group (circles) that were challenged with a second 5×10⁵ dose of B16F10 melanoma tumor cells on day 75 post initial adoptive T cell transfer 7 days after re-challenge (FIG. 5A) and 14 days after re-challenge (FIG. 5B), where 4 or 5 mice were observed for each group (n=4 or 5).

FIG. 5C is a graph showing the number of CD8+ T cells with intracellular cytokine levels of IFN+ (bottom of each column), TNF+ (middle of each column), and both IFN+ and TNF+ (top of each column) for cells that were pulsed with Trp1 and Trp2 peptides before staining and were collected from mice 14 days after the mice were challenged with a second 5×10⁵ dose of B16F110 melanoma tumor cells on day 75 post initial adoptive T cell transfer with 1×10⁶ pmel T cells and 10 μg of amphiphilic gp100 or 5×10⁶ pmel T cells and 10 μg of amphiphilic gp100 vaccine in comparison to a tumor naïve control group, where 4 or 5 mice were observed for each group (n=4 or 5).

FIG. 6A is a graph showing the survival of mice that had previously rejected a tumor following adoptive T cell transfer of 1×10⁶ pmel T cells (dashed line) or 5×10⁶ pmel T cells (rightmost, solid line) vaccinated with 10 μg of amphiphilic gp100 in comparison to a control (leftmost, solid line dropping to 0% survival before day 20) after the mice had been challenged 2× higher dose of 1×10⁶ B16F110 melanoma tumor cells 75 days after initial adoptive transfer and 82 days after first being injected with tumor cells, where 4 or 5 mice were observed for each group (n=4 or 5).

FIG. 6B is a graph showing the tumor volume in mice over time that had previously rejected tumor following adoptive T cell transfer of 1×10⁶ pmel T cells (dashed lines) or 5×10⁶ pmel T cells (rightmost, solid lines) vaccinated with 10 μg of amphiphilic gp100 in comparison to a control (solid lines to the left of the dashed lines) after the mice had been challenged 2× higher doses of 1×10⁶ B116F110 melanoma tumor cells 75 days after initial adoptive transfer and 82 days after first being injected with tumor cells, where 4 or 5 mice were observed for each group (n=4 or 5).

FIG. 7 is a graph showing the number of pmel T cells in the tumor cells of mice, who were administered 5×10⁵ B116F10 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 8A and FIG. 8B are graphs showing the number of CD8+ T cells (FIG. 8A) and the ratio of CD8+:CD4+ T cells (FIG. 8B) in the tumor cells of mice, who were administered 5×10⁵ B116F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 9 is a graph showing the number of CD8+, CD25+ T cells in the tumor cells of mice, who were administered 5×10⁵ B116F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 10 is a graph showing the number of PD-1+(bottom of each column); PD-1+ and TIM3+ or LAG3+(middle of each column); and PD-1+, TIM3+, and LAG3+(top of each column) in the tumor cells of mice, who were administered 5×10⁵ B116F10 melanoma tumor cells on day −7, 7 days after being administered PBS, soluble gp100, amphiphilic gp100, 1×10⁶ pmel T cells (leftmost, squares), 1×10⁶ pmel T cells and soluble gp100, 1×10⁶ pmel T cells and amphiphilic gp100, 5×10⁶ pmel T cells, 5×10⁶ pmel T cells and soluble gp100, or 5×10⁶ pmel T cells and amphiphilic gp100, where each was observed in 9 or 10 mice (n=9 or 10).

FIG. 11 is a graph showing the number of pmel T cells that are naïve, central memory (CM), or effector (Eff) T cells in the tumor cells of mice, who were administered 5×10⁵ B16F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 12A is graph showing number of Ki67+ and CD8+ pmel T cells in the tumor cells of mice, who were administered 5×10⁵ B116F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 12B is graph showing the number of CD8+ pmel T cells that show IFN+(bottom of each column), GrzB+ (middle of each column), and IFN+ and GrzB+ (top of each column) cytokine secretion in the tumor cells of mice, who were administered 5×10⁵ B16F110 melanoma tumor cells on day −7, 7 days after being administered PBS, soluble gp100, amphiphilic gp100, 1×10⁶ pmel T cells, 1×10⁶ pmel T cells and soluble gp100, 1×10⁶ pmel T cells and amphiphilic gp100, 5×10⁶ pmel T cells, 5×10⁶ pmel T cells and soluble gp100, or 5×10⁶ pmel T cells and amphiphilic gp100, where each was observed in 9 or 10 mice (n=9 or 10).

FIG. 13 is graph showing the number of CD8+ pmel T cells, that were pulsed with EGP peptides, that show IFN+(bottom of each column), GrzB+ (middle of each column), and IFN+ and GrzB+ (top of each column) cytokine secretion in the tumor cells of mice, who were administered 5×10⁵ B16F10 melanoma tumor cells on day −7, 7 days after being administered PBS, soluble gp100, amphiphilic gp100, 1×10⁶ pmel T cells, 1×10⁶ pmel T cells and soluble gp100, 1×10⁶ pmel T cells and amphiphilic gp100, 5×10⁶ pmel T cells, 5×10⁶ pmel T cells and soluble gp100, or 5×10⁶ pmel T cells and amphiphilic gp100, where each was observed in 9 or 10 mice (n=9 or 10).

FIG. 14A and FIG. 14B are graphs showing the number of CD8+ pmel T cells, that were pulsed with Trp1 peptides (FIG. 14A) or Trp2 peptides (FIG. 14B), that show IFN+ secretion (bottom of each column), GrzB+ (middle of each column), and IFN+ and GrzB+ (top of each column) cytokine secretion in the tumor cells of mice, who were administered 5×10⁵ B16F110 melanoma tumor cells on day −7, 7 days after being administered PBS, soluble gp100, amphiphilic gp100, 1×10⁶ pmel T cells, 1×10⁶ pmel T cells and soluble gp100, 1×106 pmel T cells and amphiphilic gp100, 5×106 pmel T cells, 5×106 pmel T cells and soluble gp100, or 5×106 pmel T cells and amphiphilic gp100, where each was observed in 9 or 10 mice (n=9 or 10).

FIG. 15 is a graph showing the number of pmel T cells in the peripheral blood cells of mice, who were administered 5×10⁵ B16F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 16 is a graph showing the number of CD8+ T cells in the peripheral blood cells of mice, who were administered 5×10⁵ B16F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 17 is graph showing the number of CD8+ pmel T cells, that were pulsed with EGP peptides, that show IFN+(bottom of each column), GrzB+ (middle of each column), and IFN+ and GrzB+ (top of each column) cytokine secretion in the peripheral blood cells of mice, who were administered 5×10⁵ B16F10 melanoma tumor cells on day −7, 7 days after being administered PBS, soluble gp100, amphiphilic gp100, 1×10⁶ pmel T cells, 1×10⁶ pmel T cells and soluble gp100, 1×10⁶ pmel T cells and amphiphilic gp100, 5×10⁶ pmel T cells, 5×10⁶ pmel T cells and soluble gp100, or 5×10⁶ pmel T cells and amphiphilic gp100, where each was observed in 9 or 10 mice (n=9 or 10).

FIG. 18A and FIG. 18B are graphs showing the number of CD8+ pmel T cells, that were pulsed with Trp1 peptides (FIG. 18A) or Trp2 peptides (FIG. 18B), that show IFN+(bottom of each column), TNF+ (middle of each column), and IFN+ and TNF+ (top of each column) cytokine secretion in the peripheral blood cells of mice, who were administered 5×10⁵ B116F110 melanoma tumor cells on day −7, 7 days after being administered PBS, soluble gp100, amphiphilic gp100, 1×10⁶ pmel T cells, 1×10⁶ pmel T cells and soluble gp100, 1×10⁶ pmel T cells and amphiphilic gp100, 5×10⁶ pmel T cells, 5×10⁶ pmel T cells and soluble gp100, or 5×10⁶ pmel T cells and amphiphilic gp100, where each was observed in 9 or 10 mice (n=9 or 10).

FIG. 19 is a graph showing the number of CD8+ T cells in the lymph nodes (LN) of mice, who were administered 5×10⁵ B116F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 20 is a graph showing the number of pmel T cells in the lymph nodes of mice, who were administered 5×10⁵ B116F110 melanoma tumor cells on day −7, 7 days after being administered PBS (leftmost, circles), soluble gp100 (middle, circles), amphiphilic gp100 (rightmost, circles), 1×10⁶ pmel T cells (leftmost, squares), 1×10⁶ pmel T cells and soluble gp100 (middle squares), 1×10⁶ pmel T cells and amphiphilic gp100 (rightmost, squares), 5×10⁶ pmel T cells (leftmost, triangles), 5×10⁶ pmel T cells and soluble gp100 (middle, triangles), or 5×10⁶ pmel T cells and amphiphilic gp100 (rightmost, triangles), where each was observed in 9 or 10 mice (n=9 or 10).

FIG. 21A is a graph showing percentage of CD25+, CD69+, and CD25+ and CD69+ T cells that were also CD8+ T cells in naïve pmel T cells that were isolated from splenocytes of mice and cocultured with dendritic cells (DC2.4) that were labelled with nothing (leftmost in % CD25, % CD69, and % CD25, CD69 groups), labelled with soluble gp100 (middle in % CD25, % CD69, and % CD25, CD69 groups), or labelled with amphiphilic gp100 (leftmost in % CD25, % CD69, and % CD25, CD69 groups).

FIG. 21B is a graph showing percent lysis of tumor cells expressing firefly luciferase when co-cultured with adoptively transferred T cells at various effector-to-target (E:T) ratios in triplicate, where, from bottom of the graph to top of the graph, the T cells were unactivated, the T cells were co-cultured with DC2.4 wild-type cells, the T cells were cocultured with DC2.4 cells labeled with soluble gp100, or the T cells were cocultured with DC2.4 cells labeled with amphiphilic gp100.

FIG. 22A is a graph showing the percentage of T cells spinoculated with viral supernatant collected from mCherry transfected Phoenix-ECO cells that were activated with CD25, CD69, or CD25 and CD69 after being cocultured with wild-type DC 2.4, DC 2.4 cells labeled with soluble gp100, or DC 2.4 cells labeled with amphiphilic gp100. For the mCherry, pmel T cells on day 0 (leftmost column) the percentage of T cells with CD25 is on the bottom of the column and the percentage with CD69 is on top. For the mCherry, pmel T cells cocultured with DC 2.4 labeled with soluble gp100, the percentage of T cells with CD25 is on the bottom of the column and the percentage with both CD25 and CD69 is on the top of the column. For the mCherry, pmel T cells coculture with DC 2.4 labeled with amphiphilic gp100, the percentage of T cells with CD25 is on the bottom of the column, the percentage of cells with CD69 is in the middle of the column, and the percentage of T cells with both CD25 and CD69 is on the top of the column.

FIG. 22B is a graph showing the percent lysis of tumor cells expressing firefly luciferase when co-cultured with adoptively transferred T cells spinoculated with viral supernatant collected from mCherry at various effector-to-target (E:T) ratios in triplicate, where the T cells were unactivated (grey circles), the T cells were co-cultured with DC2.4 wild-type cells (black circles), the T cells were cocultured with DC2.4 cells labeled with soluble gp100 (squares), the T cells were cocultured with DC2.4 cells labeled with amphiphilic gp100 (triangles), or the T cells were cocultured with DC2.4 cells labeled with amphiphilic fluorescein isothiocyanate (FITC) (diamonds).

FIG. 23 is a graph showing dendritic cell activation in the lymph nodes of mice vaccinated with PBS (leftmost set of data), soluble (sol) gp100 (middle set of data), or amphiphilic (amp) gp100 (rightmost set of data) by measuring the mean fluorescence intensity (MFI) to analyze the activation for, from left to right in each set of data, CD40+, CD80+, CD86+, and MHC II+.

FIG. 24 is a graph of the number of pmel T cells that were isolated from splenocytes of mice and cultured in a 1:1 ratio with lymph node homogenate of mice vaccinated with PBS (black circles), soluble gp100 (squares), or amphiphilic gp100 (triangles) in comparison to pmel T cells alone (gray circles) over a period of 0 to 6 days.

FIG. 25A is graph showing the amount of IFNγ produced by unstimulated pmel T cells, pmel T cells cocultured with lymph node homogenate of mice vaccinated with PBS, soluble gp100, or amphiphilic gp100, after the pmel T cells with cocultured for 24 hours.

FIG. 25B is a graph showing the pmel T cell activation with CD25+, CD69+, or CD25+ and CD69+, in pmel T cell that were cocultured with lymph node homogenate of mice vaccinated with PBS, soluble gp100, or amphiphilic gp100 for 1, 3, or 6 days in comparison to an unstimulated pmel T cell control. The bottom section of each column indicates the percentage of CD25+ pmel T cells, the middle section indicates the percentage of CD69+ pmel Tcells, and the top section indicates the percentage of CD25+ and CD69+ pmel Tcells.

FIG. 26A is a graph showing the percent lysis of tumor cells expressing firefly luciferase when co cultured with adoptively transferred T cells at various effector-to-target (E:T) ratios in triplicate, where the T cells were unstimulated (grey circles) or the T cells were co-cultured with lymph node homogenate of mice vaccinated with PBS (black circles), soluble gp100 (squares), or amphiphilic gp100 (triangles) after the cells were cocultured for 1 day.

FIG. 26B is a series of graphs showing the amount of cytokines produced, including IFNγ, IL-2, and TNFα, as a result of co-culturing pmel T cells with lymph node homogenate of mice vaccinated with PBS (black circles), soluble gp100 (squares), or amphiphilic gp100 (triangles), in comparison to unstimulated pmel T cells (gray circles) after the cells were cocultured for 1 day.

FIG. 27A is a graph showing the percent lysis of tumor cells expressing firefly luciferase when co cultured with adoptively transferred T cells at various effector-to-target (E:T) ratios in triplicate, where the T cells were unstimulated (grey circles) or the T cells were co-cultured with lymph node homogenate of mice vaccinated with PBS (black circles), soluble gp100 (squares), or amphiphilic gp100 (triangles) after the cells were cocultured for 7 days.

FIG. 27B is a series of graphs showing the amount of cytokines produced, including IFNγ, IL-2, and TNFα, as a result of co-culturing pmel T cells with lymph node homogenate of mice vaccinated with PBS (black circles), soluble gp100 (squares), or amphiphilic gp100 (triangles), in comparison to unstimulated pmel T cells (gray circles), after the cells were cocultured for 7 days.

FIG. 28 is a graph showing the number of pmel T cells spinoculated with viral supernatant collected from mCherry transfected Phoenix-ECO cells in mice that were administered 10 μg of soluble gp100 (squares) or 10 μg of amphiphilic gp100 (triangles) in comparison to an untreated control (circles) either 5 days of 19 days after the T cell infusion, where red blood cells were collected from between 1 and 9 mice for each group (n=1-n=9).

FIG. 29A is a graph showing tumor volume in mice over time after treatment with 1×10⁶ T cells spinoculated with viral supernatant collected from mCherry transfected Phoenix-ECO cells (solid lines labelled A), 1×10⁶ T cells and soluble gp100 (solid lines labelled B), or 1×10⁶ T cells and amphiphilic gp100 (solid lines labelled C), where 10 mice were observed for each (n=10).

FIG. 29B is a graph showing the percentage of mouse survival over time after being injected with 5×10⁵ B16F10 melanoma tumor cells for mice who were administered 1×10⁶ mCherry transduced pmel T cells and PBS (leftmost, solid line), 1×10⁶ mCherry transduced pmel T cells and 10 μg of soluble gp100 (middle, solid line), or 1×10⁶ mCherry transduced pmel T cells and 10 μg of amphiphilic gp100 (rightmost, solid line), where 10 mice were observed for each group (n=10).

FIG. 29C is a graph showing the percentage of survival in mice over time after treatment with 1×10⁶ T cells spinoculated with viral supernatant collected from mCherry transfected Phoenix-ECO cells (solid lines labelled A), 1×10⁶ T cells and soluble gp100 (solid lines labelled B), or 1×10⁶ T cells and amphiphilic gp100 (solid lines labelled C), where 10 mice were observed for each (n=10).

FIG. 30 is a graph showing the number of Thy1.1+ and CD8+ T cells in mice that were administered PBS (leftmost circles), 10 μg of soluble gp100 (middle, circles), 10 μg of amphiphilic gp100 (rightmost, circles), 1×10⁵ pmel T cells (leftmost, squares), 1×10⁵ pmel T cells and 10 μg of soluble gp100 (middle, squares), 1×10⁵ pmel T cells and 10 μg of amphiphilic gp100 (rightmost, squares), 1×10⁶ pmel T cells (leftmost, triangles), 1×10⁶ pmel T cells and 10 μg of soluble gp100 (middle, triangles), or 1×10⁶ pmel T cells and 10 μg of amphiphilic gp100 (leftmost, triangles), where red blood cells were collected from 5 mice for each group (n=5).

FIG. 31 is a graph showing the number of Thy1.1+ T cells in mice that were administered PBS (leftmost circles), 10 μg of soluble gp100 (middle, circles), 10 μg of amphiphilic gp100 (rightmost, circles), 1×10⁵ pmel T cells (leftmost, squares), 1×10⁵ pmel T cells and 10 μg of soluble gp100 (middle, squares), 1×10⁵ pmel T cells and 10 μg of amphiphilic gp100 (rightmost, squares), 1×10⁶ pmel T cells (leftmost, triangles), 1×10⁶ pmel T cells and 10 μg of soluble gp100 (middle, triangles), or 1×10⁶ pmel T cells and 10 μg of amphiphilic gp100 (leftmost, triangles), where red blood cells were collected from 5 mice for each group (n=5).

FIG. 32A is a graph showing the tumor volume in mice over time after receiving after vaccination with PBS (dotted lines and circles), soluble gp100 (dotted lines and squares), amphiphilic gp100 (dotted lines and triangles), 1×10⁵ pmel T cells (dashed lines and circles), 1×10⁵ T cells and soluble gp100 (dashed lines and squares), 1×10⁵ T cells and amphiphilic gp100 (dashed lines and triangles), 1×10⁶ pmel T cells (solid lines and circles), 1×10⁶ T cells and soluble gp100 (solid lines and squares), 1×10⁶ T cells and amphiphilic gp100 (solid lines and triangles), where 10 mice were observed for each (n=10) and where the mice were treated with whole body irradiation prior to vaccination.

FIG. 32B is a graph showing the percentage of mouse survival over time after being injected with 5×10⁵ B16F10 melanoma tumor cells for mice who were administered PBS (leftmost, dotted line), 10 μg of soluble gp100 (rightmost, dotted line), 10 μg of amphiphilic gp100 (middle, dotted line), 1×10⁵ pmel T cells (rightmost, dashed line), 1×10⁵ pmel T cells and 10 μg of soluble gp100 (middle, dashed line), 1×10⁵ pmel T cells and 10 μg of amphiphilic gp100 (rightmost, dashed line), 1×10⁶ pmel T cells (leftmost, solid line), 1×10⁶ pmel T cells and 10 μg of soluble gp100 (leftmost, solid line), or 1×10⁶ pmel T cells and 10 μg of amphiphilic gp100 (rightmost, solid line), where 10 mice were observed for each group (n=10) and where the mice were treated with whole body irradiation prior to vaccination.

FIG. 32C is a graph showing the percentage mouse body weight change over time after receiving vaccination with PBS (dotted lines and circles), soluble gp100 (dotted lines and squares), amphiphilic gp100 (dotted lines and triangles), 1×10⁵ pmel T cells (dashed lines and circles), 1×10⁵T cells and soluble gp100 (dashed lines and squares), 1×10⁵ T cells and amphiphilic gp100 (dashed lines and triangles), 1×10⁶ pmel T cells (solid lines and circles), 1×10⁶T cells and soluble gp100 (solid lines and squares), 1×10⁶ T cells and amphiphilic gp100 (solid lines and triangles), where 10 mice were observed for each (n=10) and where the mice were treated with whole body irradiation prior to vaccination.

FIG. 33A is a graph showing the percentage of mouse survival over time after being injected with 5×10⁵ B16F10 melanoma tumor cells on D-10 followed by DO administration of 5×10⁶ mCherry transduced pmel T cells and a PBS subcutaneous vaccination regimen (leftmost, solid line), 5×10⁶ mCherry transduced pmel T cells and 10 μg of a soluble gp100/soluble CpG subcutaneous vaccination regimen (middle, solid line), or 5×10⁶ mCherry transduced pmel T cells and 10 μg of a amphiphilic gp100/amphiphilic CpG subcutaneous vaccination regimen (rightmost, solid line), where 10 mice (n=10), 20 mice (n=20), and 24 mice (n=24) were observed for each respective group.

FIG. 33B is a series of graphs showing tumor volume in mice over time after being injected with 5×10⁵ B16F10 melanoma tumor cells on D-10 followed by DO administration of 5×10⁶ mCherry transduced pmel T cells and a PBS subcutaneous vaccination regimen (leftmost graph), 5×10⁶ mCherry transduced pmel T cells and 10 μg of a soluble gp100/soluble CpG subcutaneous vaccination regimen (middle graph), or 5×10⁶ mCherry transduced pmel T cells and 10 μg of an amphiphilic gp100/amphiphilic CpG subcutaneous vaccination regimen (rightmost graph), where 10 mice (n=10), 20 mice (n=20), and 24 mice (n=24) were observed for each respective group.

FIG. 34A is a graph showing the number of mCherry transduced pmel T cells in the peripheral blood cells of mice 5 days following administration of T cells and a PBS vaccination regimen following a 10 day 5×10⁵ B16F10 melanoma tumor implantation (leftmost, circles), 5 days following administration of T cells and a 10 μg amphiphilic gp100/amphiphilic CpG vaccination regimen following a 10 day 5×10⁵ B16F10 melanoma tumor implantation (middle, circles), or 75 days following administration of T cells and a 10 μg amphiphilic gp100/amphiphilic CpG vaccination regimen following a 10 day 5×10⁵ B16F10 melanoma tumor implantation (rightmost, circles).

FIG. 34B is a graph showing the tumor volume in untreated mice challenged with a 5×10⁵ dose of B16F10 melanoma tumor cells as a control for secondary tumor rechallenge on day 75 post initial adoptive T cell transfer.

FIG. 34C is a graph showing the tumor volume in mice which had previously rejected tumor following adoptive T cell transfer were challenged with a second 5×10⁵ dose of B16F10 melanoma tumor cells on day 75 post initial adoptive T cell transfer with 5×10⁶ mCherry transduced pmel T cells and 10 μg of amphiphilic gp100/amphiphilic CpG vaccination regimen where doses of vaccine were given two times a week for two weeks via subcutaneous tail base injection on days 3, 7, 10, and 14. 7 mice were observed for this group (n=7).

FIG. 34D is a graph showing the percentage survival of mice which had previously rejected tumor following adoptive T cell transfer were challenged with a second 5×10⁵ dose of B16F10 melanoma tumor cells on day 75 post initial adoptive T cell transfer with 5×10⁶ mCherry transduced pmel T cells and 10 μg of amphiphilic gp100/amphiphilic CpG vaccination regimen where doses of vaccine were given two times a week for two weeks via subcutaneous tail base injection on days 3, 7, 10, and 14 (top line) in comparison to a tumor naïve control group (bottom line), where 7 or 10 mice were observed for each group (n=7 or 10).

FIG. 35A is a graph showing the number of pmel T cells in the peripheral blood cells of B16F10 tumor bearing mice 5 days after T cell injection who were administered on day −1 subcutaneously PBS (leftmost circles), 10 μg soluble gp100 peptide/soluble CpG (middle circles), or 10 μg of amphiphilic gp100 peptide/amphiphilic CpG (rightmost circles) in addition to, on day 0, 5×10⁶ mCherry transduced T cells previously isolated from splenocytes of 6-8 week old pmel-1 mice, and who were administered a subsequent booster dose of vaccine given via subcutaneous tail base injection on day 3.

FIG. 35B is a graph showing the number of pmel T cells in the lymph nodes of B16F10 tumor bearing mice 7 days after T cell injection who were administered on day −1 subcutaneously PBS (leftmost circles), 10 μg soluble gp100 peptide/soluble CpG (middle circles), or 10 μg of amphiphilic gp100 peptide/amphiphilic CpG (rightmost circles) in addition to, on day 0, 5×10⁶ mCherry transduced T cells previously isolated from splenocytes of 6-8 week old pmel-1 mice, and who were administered a subsequent booster dose of vaccine given via subcutaneous tail base injection on day 3.

FIG. 35C a graph showing the number of pmel T cells in the tumor cells of mice 5 days after T cell injection who were administered on day −1 subcutaneously PBS (leftmost circles), 10 μg soluble gp100 peptide/soluble CpG (middle circles), or 10 μg of amphiphilic gp100 peptide/amphiphilic CpG (rightmost circles) in addition to, on day 0, 5×10⁶ T cells previously isolated from splenocytes of 6-8 week old pmel-1 mice, and who were administered a subsequent booster dose of vaccine given via subcutaneous tail base injection on day 3.

FIG. 36A is a graph showing the number of dendritic cells in the lymph nodes of B16F10 tumor bearing mice 7 days after T cell injection who were administered on day −1 subcutaneously PBS (leftmost circles), 10 μg soluble gp100 peptide/soluble CpG (middle circles), or 10 μg of amphiphilic gp100 peptide/amphiphilic CpG (rightmost circles) in addition to, on day 0, 5×10⁶ mCherry transduced T cells previously isolated from splenocytes of 6-8 week old pmel-1 mice, and who were administered a subsequent booster dose of vaccine given via subcutaneous tail base injection on day 3.

FIG. 36B is a graph showing the number of CD40 positive dendritic cells in the lymph nodes of B16F10 tumor bearing mice 7 days after T cell injection who were administered on day −1 subcutaneously PBS (leftmost circles), 10 μg soluble gp100 peptide/soluble CpG (middle circles), or 10 μg of amphiphilic gp100 peptide/amphiphilic CpG (rightmost circles) in addition to, on day 0, 5×10⁶ mCherry transduced T cells previously isolated from splenocytes of 6-8 week old pmel-1 mice, and who were administered a subsequent booster dose of vaccine given via subcutaneous tail base injection on day 3.

FIG. 36C is a graph showing the number of MHCII positive dendritic cells in the lymph nodes of B16F10 tumor bearing mice 7 days after T cell injection who were administered on day −1 subcutaneously PBS (leftmost circles), 10 μg soluble gp100 peptide/soluble CpG (middle circles), or 10 μg of amphiphilic gp100 peptide/amphiphilic CpG (rightmost circles) in addition to, on day 0, 5×10⁶ mCherry transduced T cells previously isolated from splenocytes of 6-8 week old pmel-1 mice, and who were administered a subsequent booster dose of vaccine given via subcutaneous tail base injection on day 3.

FIG. 36D is a graph showing the number of dendritic cells in the lymph nodes of B16F10 tumor bearing mice that were CD80 positive and CD86 negative (bottom of each column), CD80 negative and CD86 positive (middle of each column), and CD80 positive and CD86 positive (top of each column), 7 days after being administered a T cell injection on day −1 subcutaneously PBS, 10 μg soluble gp100 peptide/soluble CpG, or 10 μg of amphiphilic gp100 peptide/amphiphilic CpG, in addition to, on day 0, 5×10⁶ mCherry transduced T cells previously isolated from splenocytes of 6-8 week old pmel-1 mice, and after being administered a subsequent booster dose of vaccine given via subcutaneous tail base injection on day 3.

FIG. 37 is an image of the 561 gene immunology nanostring panel showing the RNA sequencing analysis from the lymph nodes of mice harvested on day 1 after being administered on day −1 subcutaneously 10 μg soluble gp100 peptide/soluble CpG alone, 10 μg of amphiphilic gp100 peptide/amphiphilic CpG alone, 5×10⁶ mCherry transduced T cells previously isolated from splenocytes of 6-8 week old pmel-1 mice on day 0, 5×10⁶ mCherry transduced T cells on day 0 and 10 μg soluble gp100/soluble CpG on day −1, or 5×10⁶ mCherry transduced T cells on day 0 and 10 μg of amphiphilic gp100 peptide/amphiphilic CpG on day −1.

FIG. 38A is a graph showing the number of CD25+ and CD8+ T cells per mg of tumor that were found in the tumors of B16F10 tumor bearing mice 7 days after they were administered 5×10⁶ mCherry transduced pmel T cells previously isolated from splenocytes of 6-8 week old pmel-1 mice combined with a day −1 and day 3 PBS subcutaneous vaccination (leftmost circles), day −1 and day 3 10 μg soluble gp100 peptide/soluble CpG vaccination (middle circles), or day −1 and day 3 10 μg amphiphilic gp100 peptide/amphiphilic CpG vaccination (rightmost circles).

FIG. 38B is a graph showing the number of Ki67+ and CD8+ T cells per mg of tumor that were found in the tumors of B16F10 tumor bearing mice 7 days after they were administered 5×10⁶ mCherry transduced pmel T cells previously isolated from splenocytes of 6-8 week old pmel-1 mice combined with a day −1 and day 3 PBS subcutaneous vaccination (leftmost circles), day −1 and day 3 10 μg soluble gp100 peptide/soluble CpG vaccination (middle circles), or day −1 and day 3 10 μg amphiphilic gp100 peptide/amphiphilic CpG vaccination (rightmost circles).

FIG. 38C is a graph showing the number of CD8 T cells per mg of tumor that were found in the tumors of B16F10 tumor bearing mice that were IFNγ+(bottom of each column), TNFα⁺ (middle of each column), or IFNγ+ and TNFα⁺ (top of each column) 7 days after they were administered 5×10⁶ mCherry transduced pmel T cells previously isolated from splenocytes of 6-8 week old pmel-1 mice combined with a day −1 and day 3 PBS subcutaneous vaccination (leftmost bar), day −1 and day 3 10 μg soluble gp100 peptide/soluble CpG vaccination (middle bar), or day −1 and day 3 10 μg amphiphilic gp100 peptide/amphiphilic CpG vaccination (rightmost bar).

FIG. 39 is an image of the 561 gene immunology nanostring panel showing the RNA sequencing analysis from the B16F10 tumors of mice on day 7 after DO administration of 5×10⁶ mCherry transduced pmel T cells previously isolated from splenocytes of 6-8 week old pmel-1 mice, or after DO administration of 5×10⁶ mCherry transduced pmel T cells following combination with D-1 subcutaneous vaccination with 10 μg amphiphilic gp100 peptide/amphiphilic CpG.

FIG. 40A is a graph showing the number of TRP1 specific CD8 T cells per mg of tumor that were found in the tumors of B16F10 tumor bearing mice that were IFNγ⁺ (bottom of each column), TNFα⁺ (middle of each column), or IFNγ⁺ and TNFα⁺ (top of each column), 7 days after they were administered 5×10⁶ mCherry transduced pmel T cells previously isolated from splenocytes of 6-8 week old pmel-1 mice combined with a day −1 and day 3 PBS subcutaneous vaccination (leftmost bar), day −1 and day 3 10 μg soluble gp100 peptide/soluble CpG vaccination (middle bar), or day −1 and day 3 10 μg amphiphilic gp100 peptide/amphiphilic CpG vaccination (rightmost bar).

FIG. 40B is a graph showing the number of TRP2 specific CD8 T cells per mg of tumor that were found in the tumors of mice that were IFNγ⁺ (bottom of each column), TNFα⁺ (middle of each column), or IFNγ⁺ and TNFα⁺ (top of each column), 7 days after they were administered 5×10⁶ mCherry transduced pmel T cells previously isolated from splenocytes of 6-8 week old pmel-1 mice combined with a day −1 and day 3 PBS subcutaneous vaccination (leftmost bar), day −1 and day 3 10 μg soluble gp100 peptide/soluble CpG vaccination (middle bar), or day −1 and day 3 10 μg amphiphilic gp100 peptide/amphiphilic CpG vaccination (rightmost bar).

FIG. 40C is a graph showing the number of B16 TAA specific CD8 T cells per mg of tumor that were found in the tumors of mice that were IFNγ+(bottom of each column), TNFα⁺ (middle of each column), or IFNγ⁺ and TNFα⁺ (top of each column), 7 days after they were administered 5×10⁶ mCherry transduced pmel T cells previously isolated from splenocytes of 6-8 week old pmel-1 mice combined with a day −1 and day 3 PBS subcutaneous vaccination (leftmost bar), day −1 and day 3 10 μg soluble gp100 peptide/soluble CpG vaccination (middle bar), or day −1 and day 3 10 μg amphiphilic gp100 peptide/amphiphilic CpG vaccination (rightmost bar).

FIG. 41 is a graph showing the number of mCherry transduced pmel T cells, generated from previously isolated splenocytes of 6-8 week old pmel-1 mice, over time following activation by a 1:1 24 hour culture with a lymph node homogenate from mice which were euthanized 48 hours after being administered subcutaneous vaccination with PBS (second from bottom circles), 10 μg soluble gp100 (second from top circles), or 10 μg amphiphilic gp100 (top circles). mCherry transduced pmel T cells that were not cultured with lymph node homogenate were used as a control (bottom circles).

FIG. 42 is a graph showing the percent lysis of tumor cells at various effector-to-target (E:T) ratios of mCherry transduced pmel T cells, generated from previously isolated splenocytes of 6-8 week old pmel-1 mice, activated for 24 hours at a 1:1 ratio with lymph node homogenate generated from mice subcutaneously vaccinated 48 hours prior with PBS (second from the bottom circles), 10 μg soluble gp100 (second from the top circles), or 10 μg amphiphilic gp100 (top circles). mCherry transduced pmel T cells that were not cultured with lymph node homogenate were used as a control (bottom circles).

FIG. 43A is a graph showing the amount of IFNγ produced in supernatant liquid of mCherry transduced pmel T cells, generated from previously isolated splenocytes of 6-8 week old pmel-1 mice, 1 Day following activation by a 24 hour, 1:1 culture with a lymph node homogenate from mice which were euthanized 48 hours after being administered subcutaneous vaccination with PBS (middle-left circles), 10 μg soluble gp100 (middle-right circles), or 10 μg amphiphilic gp100 (rightmost circles). mCherry transduced pmel T cells that were not cultured with lymph node homogenate were used as a control (leftmost circles).

FIG. 43B is a graph showing the amount of IFNγ produced in supernatant liquid of mCherry transduced pmel T cells, generated from previously isolated splenocytes of 6-8 week old pmel-1 mice, 7 Days following activation by a 24 hour, 1:1 culture with a lymph node homogenate from mice which were euthanized 48 hours after being administered subcutaneous vaccination with PBS (middle-left circles), 10 μg soluble gp100 (middle-right circles), or 10 μg amphiphilic gp100 (rightmost circles). mCherry transduced pmel T cells that were not cultured with lymph node homogenate were used as a control (leftmost circles).

FIG. 44 is a graph showing the number of CD25+ T cells (bottom of each column), CD69+ T cells (middle of each column), and CD25+ and CD69+ T cells (top of each column) that were found from co-culture of mCherry transduced pmel T cells, generated from previously isolated splenocytes of 6-8 week old pmel-1 mice, following activation by a 24 hour, 1:1 culture with a lymph node homogenate from mice which were euthanized 48 hours after being administered subcutaneous vaccination with PBS, 10 μg soluble gp100, or 10 μg amphiphilic gp100. mCherry transduced pmel T cells that were not cultured with lymph node homogenate were used as a control.

FIG. 45 is a graph showing the number of CD25+ T cells (bottom of each column), CD69+ T cells (middle of each column), and CD25+ and CD69+ T cells (top of each column) that were found either 1 day or 4 days following 2:1 co-culture of human T cells retrovirally transduced to express a KRAS G1 2D specific TCR (TCR701) with mature autologous dendritic cells that were previously labeled overnight for 18 hours with PBS, or amphiphilic KRAS G1 2D peptide. T cells isolated from human peripheral blood mononuclear cells were spinoculated with viral supernatant collected from Phoenix-Ampho cells transfected with the TCR 701 KRAS G12D specific TCR T Cell construct or an mCherry control construct to generate KRAS specific TCR T cells and rested for 5 days prior to culture.

FIG. 46A is a graph showing the amount of IFNγ secreted from cell cultures following 2:1 co-culture of human T cells retrovirally transduced to express a KRAS G12D specific TCR (TCR701) alone (left bars) or with mature autologous dendritic cells that were previously labeled overnight for 18 hours with PBS (middle bars), or amphiphilic KRAS G1 2D peptide (right bars). T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 46B is a graph showing the amount of IL-2 secreted from cell cultures following 2:1 co-culture of human T cells retrovirally transduced to express a KRAS G12D specific TCR (TCR701) alone (left bars) or with mature autologous dendritic cells that were previously labeled overnight for 18 hours with PBS (middle bars), or amphiphilic KRAS G1 2D peptide (right bars). T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 46C is a graph showing the amount of TNFα secreted from cell cultures following 2:1 co-culture of human T cells retrovirally transduced to express a KRAS G12D specific TCR (TCR701) alone (left bars) or with mature autologous dendritic cells that were previously labeled overnight for 18 hours with PBS (middle bars), or amphiphilic KRAS G1 2D peptide (right bars). T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 47A is a graph showing the percent lysis of Cos-7 target cells expressing luciferase gene, HLA A*11:01, and the KRAS G12D mutation at various effector to target ratios after culture with a KRAS G12D specific TCR (TCR701) alone (bottom circles) or following overnight 2:1 culture with mature autologous dendritic cells that were previously labeled overnight for 18 hours with PBS (middle circles), or amphiphilic KRAS G12D peptide (top circles). T cells isolated from human peripheral blood mononuclear cells were spinoculated with viral supernatant collected from Phoenix-Ampho cells transfected with the TCR 701 KRAS G12D specific TCR T Cell construct or an mCherry control construct to generate KRAS specific TCR T cells and rested for 5 days prior to culture.

FIG. 47B is a graph showing the percent lysis of Panc-1 human derived tumor line that also expresses a luciferase gene, HLA A*11:01, and the KRAS G12D mutation at various effector to target ratios after culture with a KRAS G12D specific TCR (TCR701) alone (bottom circles) or following overnight 2:1 culture with mature autologous dendritic cells that were previously labeled overnight for 18 hours with PBS (middle circles), or amphiphilic KRAS G12D peptide (top circles). T cells isolated from human peripheral blood mononuclear cells were spinoculated with viral supernatant collected from Phoenix-Ampho cells transfected with the TCR 701 KRAS G12D specific TCR T Cell construct or an mCherry control construct to generate KRAS specific TCR T cells and rested for 5 days prior to culture.

FIG. 48A is a graph showing the fold change of IFNγ secreted from cell cultures following 2:1 co-culture of human T cells retrovirally transduced to express a KRAS G12D specific TCR (TCR701) with mature autologous dendritic cells that were previously labeled overnight for 18 hours with freshly made soluble or amphiphilic KRAS G12D peptide (left circles), soluble KRAS G12D peptide that had been prepared 24 hours prior to labeling of dendritic cells in human serum and incubated at 37 degrees overnight to mimic in vivo conditions (bottom two lines and circles), or amphiphilic KRAS G12D peptide that had been prepared 24 hours prior to labeling of dendritic cells in human serum and incubated at 37 degrees overnight to mimic in vivo conditions (top two lines and circles). T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 48B is a graph showing the fold change in TNFα secreted from cell cultures following 2:1 co-culture of human T cells retrovirally transduced to express a KRAS G12D specific TCR (TCR701) with mature autologous dendritic cells that were previously labeled overnight for 18 hours with freshly made soluble or amphiphilic KRAS G12D peptide (left circles), soluble KRAS G12D peptide that had been prepared 24 hours prior to labeling of dendritic cells in human serum and incubated at 37 degrees overnight to mimic in vivo conditions (bottom two lines and circles), or amphiphilic KRAS G12D peptide that had been prepared 24 hours prior to labeling of dendritic cells in human serum and incubated at 37 degrees overnight to mimic in vivo conditions (top two lines and circles). T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 48C is a graph showing the fold change in IL2 secreted from cell cultures following 2:1 co-culture of human T cells retrovirally transduced to express a KRAS G12D specific TCR (TCR701) with mature autologous dendritic cells that were previously labeled overnight for 18 hours with freshly made soluble or amphiphilic KRAS G12D peptide (left circles), soluble KRAS G12D peptide that had been prepared 24 hours prior to labeling of dendritic cells in human serum and incubated at 37 degrees overnight to mimic in vivo conditions (bottom two lines and circles), or amphiphilic KRAS G12D peptide that had been prepared 24 hours prior to labeling of dendritic cells in human serum and incubated at 37 degrees overnight to mimic in vivo conditions (top two lines and circles). T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 48D is a graph showing the percent change of CD69+ and CD25+ T cells in cell cultures following 2:1 co-culture of human T cells retrovirally transduced to express a KRAS G12D specific TCR (TCR701) with mature autologous dendritic cells that were previously labeled overnight for 18 hours with soluble KRAS G12D peptide that had been prepared 24 hours prior to labeling of dendritic cells in human serum and incubated at 37 degrees overnight to mimic in vivo conditions in comparison to freshly made soluble KRAS G12D peptide (left bar), or amphiphilic KRAS G12D peptide that had been prepared 24 hours prior to labeling of dendritic cells in human serum and incubated at 37 degrees overnight to mimic in vivo conditions compared to freshly prepared amphiphilic KRAS G12D peptide (right bar). T cells isolated from human peripheral blood mononuclear cells were spinoculated with viral supernatant collected from Phoenix-Ampho cells transfected with the TCR 701 KRAS G12D specific TCR T Cell construct or an mCherry control construct to generate KRAS specific TCR T cells and rested for 5 days prior to culture.

FIG. 48E is a graph showing the fold change in specific lysis of cell cultures following 2:1 co-culture of human T cells retrovirally transduced to express a KRAS G12D specific TCR (TCR701) with mature autologous dendritic cells that were previously labeled overnight for 18 hours with freshly made soluble or amphiphilic KRAS G12D peptide (left circles), soluble KRAS G12D peptide that had been prepared 24 hours prior to labeling of dendritic cells in human serum and incubated at 37 degrees overnight to mimic in vivo conditions (bottom two lines and circles), or amphiphilic KRAS G12D peptide that had been prepared 24 hours prior to labeling of dendritic cells in human serum and incubated at 37 degrees overnight to mimic in vivo conditions (top two lines and circles). T cells isolated from human peripheral blood mononuclear cells were spinoculated with viral supernatant collected from Phoenix-Ampho cells transfected with the TCR 701 KRAS G12D specific TCR T Cell construct or an mCherry control construct to generate KRAS specific TCR T cells and rested for 5 days prior to culture.

FIG. 49A is a graph showing the number of transduced T cells in the peripheral blood cells of tumor naïve mice that were administered a 5×10⁵ dose of B16F10 melanoma tumor cells after previously being administered an intraperitoneal injection of an anti-thy1.1 antibody one week prior (leftmost circles), mice that were administered a 5×10⁵ dose of B16F10 melanoma tumor cells 68 day prior (middle circles), and mice that were administered a second 5×10⁵ dose of B16F10 melanoma tumor cells on day 75 post initial adoptive transfer and had been administered an intraperitoneal injection of an anti-thy1.1 antibody one week prior (rightmost circles).

FIG. 49B is a graph showing the percentage survival of mice which had previously rejected tumor following adoptive T cell transfer and were challenged with a second 5×10⁵ dose of B16F10 melanoma tumor cells on day 75 post initial adoptive T cell transfer with 5×10⁶ transduced pmel T cells and 10 μg of amphiphilic gp100. Doses of anti-thy1.1 antibody were given by tail base injection on days −7, 6, 13, 20, 27, and 34 (top line; n=3) in comparison to a control group that had not been administered an initial 5×10⁵ dose of B16F10 melanoma tumor cells (bottom line; n=5).

FIG. 49C is a graph showing the tumor volume in mice which had previously rejected tumor following adoptive T cell transfer and were challenged with a second 5×10⁵ dose of B16F10 melanoma tumor cells on day 75 post initial adoptive T cell transfer with 5×10⁶ transduced pmel T cells and 10 μg of amphiphilic gp100/amphiphilic CpG vaccination regimen. Doses of anti-thy1.1 antibody were given by tail base injection on days −7, 6, 13, 20, 27, and 34.

FIG. 49D is a graph showing the tumor volume in untreated mice challenged with a 5×10⁵ dose of B16F10 melanoma tumor cells as a control for secondary tumor rechallenge on day 75 post initial adoptive T cell transfer.

FIG. 50A is a graph showing the number of mCherry+ CD3+ T cells isolated from the peripheral blood of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 50B is a graph showing the number of mCherry+CD3+ T cells that were CD25+(bottom of each column), CD69+(middle of each column), and CD25+ and CD69+(top of each column) that were isolated from the peripheral blood of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 51A is a graph showing the number of spot forming cells (SFC) per 1×10⁶ splenocytes in C57BL/6 HLA A1101 mice 16 days after they were administered PBS, or a soluble or amphiphilic G12D KRAS peptide on day −1 as well as 3×10⁶ T cells previously isolated from splenocytes of 6-8 week old C57BL/6 HLA A1101 mice and retrovirally transduced with a G12D KRAS specific TCR construct (TCR6) on day 0. The mice were also administered a subsequent booster dose of vaccine on days 3, 7, 10 and 14.

FIG. 51B is a graph showing the number of SFC per 1×10⁶ splenocytes splenocytes in C57BL/6 HLA A1101 mice 16 days after they were administered PBS, or a soluble or amphiphilic G12V KRAS peptide on day −1 as well as 3×10⁶ T cells previously isolated from splenocytes of 6-8 week old C57BL/6 HLA A1101 mice and retrovirally transduced with a G12V KRAS specific TCR construct (TCR3) on day 0. The mice were also administered a subsequent booster dose of vaccine on days 3, 7, 10 and 14.

FIG. 52 is a graph showing the number of dendritic cells (DCs) isolated from the lymph nodes of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 53A is a graph showing the number of MHCII positive DCs isolated from the lymph nodes of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 53B is a graph showing the number of CD40 positive DCs isolated from the lymph nodes of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 53C is a graph showing the number of DCs isolated from the lymph nodes of mice that were CD80+ and CD86− T cells (bottom of each column), CD80− and CD86+ T cells (middle of each column), and CD80+ and CD86+ T cells (top of each column) that were found in C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 54A is a graph showing the number of mCherry+CD3+ T cells isolated from the lymph nodes of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G1 2D KRAS specific TCR construct (TCR6) or a G12V KRAS specific TCR construct (TCR3) on day 0. The mice were also administered a subsequent booster dose of vaccine on days 3, 7, 10 and 14.

FIG. 54B is a graph showing the number of mCherry+CD3+ T cells isolated from the lymph nodes of mice that were CD25+(bottom of each column), CD69+(middle of each column), and CD25+ and CD69+(top of each column) 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 55 is a graph showing the number of DCs isolated from the lungs of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 56A is a graph showing the number of CD40+ DCs isolated from the lungs of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 56B is a graph showing the number of MHCII+ DCs isolated from the lungs of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 56C is a graph showing the number of DCs isolated from the lungs of mice that were CD80+(bottom of each column), CD86+(middle of each column), and CD80+ and CD86+(top of each column) 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 57 is a graph showing the number of mCherry+CD3+ T cells isolated from the lungs of C57BL/6 HLA A1101 mice 16 days after they were administered PBS, or a soluble or amphiphilic G12D or G12V KRAS peptide on day −1 as well as 3×10⁶ T cells previously isolated from splenocytes of 6-8 week old C57BL/6 HLA A1101 mice and retrovirally transduced with either a G12D KRAS specific TCR construct (TCR6) or a G12V KRAS specific TCR construct (TCR3) on day 0. The mice were also administered a subsequent booster dose of vaccine on days 3, 7, 10 and 14.

FIG. 58 is a graph showing the number of CD25+ T cells (bottom of each column), CD69+ T cells (middle of each column), and CD25+ and CD69+ T cells (top of each column) that were found either 1 day or 4 days following 2:1 co-culture of human T cells retrovirally transduced to express a HLA C*08:02 Restricted KRAS G12D specific TCR (TCR4095) with mature autologous dendritic cells that were previously labeled overnight for 18 hours with PBS, or amphiphilic KRAS G12D peptide. T cells isolated from human peripheral blood mononuclear cells were spinoculated with viral supernatant collected from Phoenix-Ampho cells transfected with the TCR 4095 KRAS G12D specific TCR T Cell construct or an mCherry control construct to generate KRAS specific TCR T cells and rested for 5 days prior to culture.

FIG. 59 is a graph showing the number of CD25+ T cells (bottom of each column), CD69+ T cells (middle of each column), and CD25+ and CD69+ T cells (top of each column) that were found either 1 day or 4 days following 2:1 co-culture of human T cells retrovirally transduced to express a HLA A*11:01 restricted KRAS G12V specific TCR (TCR700) with mature autologous dendritic cells that were previously labeled overnight for 18 hours with PBS, or amphiphilic KRAS G12V peptide. T cells isolated from human peripheral blood mononuclear cells were spinoculated with viral supernatant collected from Phoenix-Ampho cells transfected with the TCR 700 KRAS G1 2V specific TCR T Cell construct or an mCherry control construct to generate KRAS specific TCR T cells and rested for 5 days prior to culture.

FIG. 60A is a graph showing the amount of IFNγ secreted from cell cultures following 2:1 co-culture of human T cells retrovirally transduced to express a HLA A*11:01 restricted KRAS G12V specific TCR (TCR700) alone (left bars) or with mature autologous dendritic cells that were previously labeled overnight for 18 hours with PBS (middle bars), or amphiphilic KRAS G1 2V peptide (right bars). T cells isolated from human peripheral blood mononuclear cells were spinoculated with viral supernatant collected from Phoenix-Ampho cells transfected with the TCR 700 KRAS G12V specific TCR T Cell construct or an mCherry control construct to generate KRAS specific TCR T cells and rested for 5 days prior to culture.

FIG. 60B is a graph showing the amount of IL-2 secreted from cell cultures following 2:1 co-culture of human T cells retrovirally transduced to express a HLA A*11:01 restricted KRAS G12V specific TCR (TCR700) alone (left bars) or with mature autologous dendritic cells that were previously labeled overnight for 18 hours with PBS (middle bars), or amphiphilic KRAS G1 2V peptide (right bars). T cells isolated from human peripheral blood mononuclear cells were spinoculated with viral supernatant collected from Phoenix-Ampho cells transfected with the TCR 700 KRAS G12V specific TCR T Cell construct or an mCherry control construct to generate KRAS specific TCR T cells and rested for 5 days prior to culture.

FIG. 60C is a graph showing the amount of TNFα secreted from cell cultures following 2:1 co-culture of human T cells retrovirally transduced to express a HLA A*11:01 restricted KRAS G12V specific TCR (TCR700) alone (left bars) or with mature autologous dendritic cells that were previously labeled overnight for 18 hours with PBS (middle bars), or amphiphilic KRAS G1 2V peptide (right bars). T cells isolated from human peripheral blood mononuclear cells were spinoculated with viral supernatant collected from Phoenix-Ampho cells transfected with the TCR 700 KRAS G12V specific TCR T Cell construct or an mCherry control construct to generate KRAS specific TCR T cells and rested for 5 days prior to culture.

FIG. 61 is a graph showing the percent lysis of Cos-7 target cells expressing luciferase gene, HLA A*11:01, and the KRAS G12V mutation at various effector to target ratios after culture with a KRAS G12V specific TCR (TCR700) alone (bottom circles) or following overnight 2:1 culture with mature autologous dendritic cells that were previously labeled overnight for 18 hours with PBS (middle circles), or amphiphilic KRAS G12V peptide (top circles). T cells isolated from human peripheral blood mononuclear cells were spinoculated with viral supernatant collected from Phoenix-Ampho cells transfected with the TCR 700 KRAS G12V specific TCR T Cell construct or an mCherry control construct to generate KRAS specific TCR T cells and rested for 5 days prior to culture.

FIG. 62A is a graph showing the fold change in the TCR-T activation from cell cultures following 2:1 co-culture of human T cells retrovirally transduced to express a HLA A*11:01 restricted KRAS G1 2V specific TCR (TCR700) with mature autologous dendritic cells that were previously labeled overnight for 18 hours with freshly made soluble or amphiphilic KRAS G12V peptide (left circles), soluble KRAS G12V peptide that had been prepared 24 hours prior to labeling of dendritic cells in human serum and incubated at 37 degrees overnight to mimic in vivo conditions (bottom line and circle), or amphiphilic KRAS G12V peptide that had been prepared 24 hours prior to labeling of dendritic cells in human serum and incubated at 37 degrees overnight to mimic in vivo conditions (top line and circle). T cells isolated from human peripheral blood mononuclear cells were spinoculated with viral supernatant collected from Phoenix-Ampho cells transfected with the TCR 700 KRAS G1 2V specific TCR T Cell construct or an mCherry control construct to generate KRAS specific TCR T cells and rested for 5 days prior to culture.

FIG. 62B is a graph showing the fold change in the TCR-T tumor lysis from cell cultures following 2:1 co-culture of human T cells retrovirally transduced to express a HLA A*11:01 restricted KRAS G12V specific TCR (TCR700) with mature autologous dendritic cells that were previously labeled overnight for 18 hours with freshly made soluble or amphiphilic KRAS G12V peptide (left circles), soluble KRAS G12V peptide that had been prepared 24 hours prior to labeling of dendritic cells in human serum and incubated at 37 degrees overnight to mimic in vivo conditions (bottom line and circle), or amphiphilic KRAS G12V peptide that had been prepared 24 hours prior to labeling of dendritic cells in human serum and incubated at 37 degrees overnight to mimic in vivo conditions (top line and circle). T cells isolated from human peripheral blood mononuclear cells were spinoculated with viral supernatant collected from Phoenix-Ampho cells transfected with the TCR 700 KRAS G1 2V specific TCR T Cell construct or an mCherry control construct to generate KRAS specific TCR T cells and rested for 5 days prior to culture.

FIG. 63 is a graph showing the number of CD25+ T cells (bottom of each column), CD69+ T cells (middle of each column), and CD25+ and CD69+ T cells (top of each column) that were found either 2 days or 5 days following 2:1 co-culture of human T cells retrovirally transduced to express a HLA A*02:01 restricted E7 specific TCR (TCR1 G4) with mature autologous dendritic cells that were previously labeled overnight for 18 hours with PBS, or amphiphilic E7 peptide. T cells isolated from human peripheral blood mononuclear cells were spinoculated with viral supernatant collected from Phoenix-Ampho cells transfected with the E7 specific TCR T Cell construct or an mCherry control construct to generate E7 specific TCR T cells and rested for 5 days prior to culture.

FIG. 64A is a graph showing the amount of IFNγ secreted from cell cultures following 2:1 co-culture of human T cells retrovirally transduced to express a E7 specific TCR (TCR1 G4) alone (left bars) or with mature autologous dendritic cells that were previously labeled overnight for 18 hours with PBS (middle bars), or amphiphilic E7 peptide (right bars). T cells isolated from human peripheral blood mononuclear cells were spinoculated with viral supernatant collected from Phoenix-Ampho cells transfected with the E7 specific TCR T Cell construct or an mCherry control construct to generate E7 specific TCR T cells and rested for 5 days prior to culture.

FIG. 64B is a graph showing the amount of IL-2 secreted from cell cultures following 2:1 co-culture of human T cells retrovirally transduced to express a E7 specific TCR (TCR1 G4) alone (left bars) or with mature autologous dendritic cells that were previously labeled overnight for 18 hours with PBS (middle bars), or amphiphilic E7 peptide (right bars). T cells isolated from human peripheral blood mononuclear cells were spinoculated with viral supernatant collected from Phoenix-Ampho cells transfected with the E7 specific TCR T Cell construct or an mCherry control construct to generate E7 specific TCR T cells and rested for 5 days prior to culture.

FIG. 65 is a graph showing the percent lysis of Ca Ski target cells expressing luciferase gene, HLA A*02:01, and the HPV16 E7 epitope at various effector to target ratios after culture with a E7 specific TCR (TCR1 G4) alone (bottom circles) or following overnight 2:1 culture with mature autologous dendritic cells that were previously labeled overnight for 18 hours with PBS (middle circles), or amphiphilic E7 peptide (top circles). T cells isolated from human peripheral blood mononuclear cells were spinoculated with viral supernatant collected from Phoenix-Ampho cells transfected with the E7 specific TCR T Cell construct or an mCherry control construct to generate E7 specific TCR T cells and rested for 5 days prior to culture.

FIG. 66 is a graph showing percent lysis of tumor isolated from the splenocytes of C57BL/6 HLA A1101 mice 15 days after they were administered splenocytes of 6-8 week old C57BL/6 HLA A1101 that were pulsed with soluble (SOL) KRAS G12V peptide, amphiphilic (AMP) KRAS G12V, or a PBS control and labeled fluorescent carboxyfluorescein succinimidyl ester (CFSE). Triple asterisks denote statistical significance at a level of p=0.0005, and double asterisks denote statistical significance at a level of p=0.0018.

FIG. 67 is a graph showing fold change in the number of T cells 1, 2, 5, and 8 days following 2:1 co-culture of human T cells retrovirally transduced to express a HLA A*11:01 Restricted KRAS G12D specific TCR (TCR701) with mature autologous dendritic cells that were previously labeled overnight for 18 hours with PBS, soluble (SOL) or amphiphilic (AMP) KRAS G1 2D peptide. T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture. Quadruple asterisks denote statistical significance at a level of p<0.0001.

FIG. 68 is a graph showing number of human TCR T cells in the peripheral blood of 10 day Panc-1 (HLA A11+, KRAS G12D+) tumor bearing NSG mice 3 days following infusion with human T cells retrovirally transduced to express a HLA A*11:01 Restricted KRAS G12D specific TCR (TCR701) that were co-cultured in a ratio of with mature autologous dendritic cells that were previously labeled overnight for 18 hours with PBS, or amphiphilic (AMP) KRAS G1 2D peptide. The T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 69A is a graph showing the tumor mass in 10 day Panc-1 (HLA A11+, KRAS G12D+) tumor bearing NSG mice 35 days following infusion with human T cells retrovirally transduced to express a HLA A*11:01 Restricted KRAS G12D specific TCR (TCR701) that were co-cultured in a ratio of with mature autologous dendritic cells that were previously labeled overnight for 18 hours with PBS, or amphiphilic (AMP) KRAS G12D peptide. The T cells isolated from human peripheral blood mononuclear cells were spinoculated with viral supernatant collected from Phoenix-Ampho cells transfected with the TCR 701 KRAS G12D specific TCR T Cell construct or an mCherry control construct to generate KRAS specific TCR T cells and rested for 5 days prior to culture.

FIG. 69B is a graph showing number human TCR T cells found in the tumors of 10 day Panc-1 (HLA A11+, KRAS G12D+) tumor bearing NSG mice 35 days following infusion with human T cells retrovirally transduced to express a HLA A*11:01 Restricted KRAS G1 2D specific TCR (TCR701) that were co-cultured in a ratio of with mature autologous dendritic cells that were previously labeled overnight for 18 hours with PBS, or amphiphilic (AMP) KRAS G1 2D peptide. The T cells isolated from human peripheral blood mononuclear cells were spinoculated with viral supernatant collected from Phoenix-Ampho cells transfected with the TCR 701 KRAS G1 2D specific TCR T Cell construct or an mCherry control construct to generate KRAS specific TCR T cells and rested for 5 days prior to culture.

FIG. 70A is a graph showing the number of CD25+(bottom of each column), CD69+(middle of each column), and CD25+ and CD69+(top of each column) human TCR T Cells found on day 35 post infusion within Panc-1 (HLA A11+, KRAS G12D+) tumors that were implanted in NSG mice 10 days prior to T cell therapy. Human T cells were retrovirally transduced to express a HLA A*11:01 Restricted KRAS G12D specific TCR (TCR701) and co-cultured in a ratio of 2:1 with mature autologous dendritic cells that were previously labeled overnight for 18 hours with PBS, or amphiphilic (AMP) KRAS G12D peptide. Following in vitro co-culture, human T cells were isolated by negative bead selection prior to infusion into the 10-day Panc-1 tumor bearing NSG mice. 35 days following T cell infusion, the mice were euthanized and tumors were mechanically dissociated and analyzed by flow cytometry.

FIG. 70B is a graph showing the number of PD-1 positive (left column of each quadrant), double positive (middle column of each quadrant), and PD-1, TIM3, and LAG3 positive (right column of each quadrant) human TCR T Cells found on day 35 post infusion within Panc-1 (HLA A11+, KRAS G12D+) tumors that were implanted in NSG mice 10 days prior to T cell therapy. Human T cells were retrovirally transduced to express a HLA A*11:01 Restricted KRAS G12D specific TCR (TCR701) and co-cultured in a ratio of 2:1 with mature autologous dendritic cells that were previously labeled overnight for 18 hours with PBS, or amphiphilic (AMP) KRAS G1 2D peptide. Following in vitro co-culture, human T cells were isolated by negative bead selection prior to infusion into the 10-day Panc-1 tumor bearing NSG mice. 35 days following T cell infusion, the mice were euthanized and tumors were mechanically dissociated and analyzed by flow cytometry.

FIG. 71 is a graph showing the percentage of total T cells that are CD25+ T cells (bottom of each column), CD69+ T cells (middle of each column), and CD25+ and CD69+ T cells (top of each column) that were found 2 days following 2:1 co-culture of human T cells retrovirally transduced to express a HLA A*02:01 restricted NY-ESO-1 specific TCR (TCR1 G4) with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 72A is a graph showing the amount of IFNγ secreted from cells 2 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 72B is a graph showing the amount of TNFα secreted from cells 2 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 72C is a graph showing the amount of IL-2 secreted from cells 2 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 72D is a graph showing the amount of GM-CSF (granulocyte-macrophage colony-stimulating factor) secreted from the cell 2 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 73A is a graph showing the amount of IFNγ secreted from cells 8 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 73B is a graph showing the amount of TNFα secreted from cells 8 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 73C is a graph showing the amount of IL-2 secreted from cells 8 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 73D is a graph showing the amount of GM-CSF secreted from the cell 8 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble or amphiphilic NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. SOL=soluble labeling and AMP=amphiphile labeling.

FIG. 74 is a graph showing the percent lysis of A375 human derived tumor line expressing a luciferase gene, HLA A*02:01, and the NY-ESO-1 tumor cells at various effector to target ratios after culture with a NY-ESO-1 specific TCR (TCR1 G4) alone or following overnight 2:1 culture with mature autologous dendritic cells that were previously labeled overnight for 18 hours with PBS, soluble (SOL) NY-ESO-1 peptide, or amphiphilic (AMP) NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture.

FIG. 75 is a graph showing fold change in the number of T cells 1, 2, 5, and 8 days following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble (SOL) or amphiphilic (AMP) NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture.

FIG. 76A is a graph showing the fold change in the TCR-T activation from cell cultures over 24 hours following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble (SOL) or amphiphilic (AMP) NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. Asterisk denotes statistical significance at a level of p=0.0286.

FIG. 76B is a graph showing the fold change in the TCR-T tumor lysis from cell cultures over 24 hours following transfer of human T cells retrovirally transduced to express a HLA A*02:01 restricted NY-ESO-1 specific TCR (TCR1 G4) co-cultured in a ratio of 2:1 with mature autologous dendritic cells that were previously labeled overnight for 18 hours with PBS, soluble (SOL) or amphiphilic (AMP) NY-ESO-1 peptide. T cells isolated from human peripheral blood mononuclear cells were spinoculated with viral supernatant collected from Phoenix-Ampho cells transfected with the NY-ESO-1 specific TCR T Cell construct or an mCherry or MART-1 DMF5 TCR T control construct to generate NY-ESO-1 specific TCR T cells and rested for 5 days prior to culture. Quadruple asterisks denote statistical significance at a level of p<0.0001.

FIG. 77 is a graph showing fold change in the number of T cells 1, 2, 5, and 8 days following 2:1 co-culture of human T cells retrovirally transduced to express a HLA A*02:01 restricted Human Papilloma Virus (HPV) 16 E7 specific TCR (TCRE7) alone or with mature autologous dendritic cells that were previously labeled overnight for 18 hours with PBS, soluble E7 peptide (solE7), or amphiphilic E7 peptide (ampE7). T cells isolated from human peripheral blood mononuclear cells were spinoculated with viral supernatant collected from Phoenix-Ampho cells transfected with the E7 specific TCR T Cell construct or an mCherry control construct to generate E7 specific TCR T cells and rested for 5 days prior to culture.

### DEFINITIONS

Terms used in the claims and specification are defined as set forth below unless otherwise specified.

It must be noted that, as used in the specification and the appended claims, the singular forms “a,” “an,” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide. In some embodiments, the adjuvant is an immunostimulatory oligonucleotide as described herein. In some embodiments, the adjuvant is administered prior to, concurrently, or after administration of an amphiphilic ligand conjugate, or composition comprising the conjugate. In some embodiments, the adjuvant is co-formulated in the same composition as an amphiphilic ligand conjugate.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g., by insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, “amphiphile” or “amphiphilic” refers to a conjugate comprising a hydrophilic head group and a hydrophobic tail, thereby forming an amphiphilic conjugate. In some embodiments, an amphiphile conjugate comprises a peptide or a ligand for a MAIT cell and one or more hydrophobic lipid tails, referred to herein as an “amphiphilic ligand conjugate.” In some embodiments, the amphiphile conjugate further comprises a polymer (e.g., polyethylene glycol), wherein the polymer is conjugated to the one or more lipids or the peptide or a ligand for a MAIT cell.

The term “ameliorating” refers to any therapeutically beneficial result in the treatment of a disease state, e.g., cancer, including prophylaxis, lessening in the severity or progression, remission, or cure thereof.

The term “antigen presenting cell” or “APC” is a cell that displays foreign antigen complexed with MHC on its surface. T cells recognize this complex using T cell receptor (TCR). Examples of APCs include, but are not limited to, dendritic cells (DCs), peripheral blood mononuclear cells (PBMC), monocytes (such as THP-1), B lymphoblastoid cells (such as CIR.A2 and 1518 B-LCL) and monocyte-derived dendritic cells (DCs). Some APCs internalize antigens either by phagocytosis or by receptor-mediated endocytosis.

As used herein, the term “antigenic formulation” or “antigenic composition” or “immunogenic composition” refers to a preparation which, when administered to a vertebrate, especially a mammal, will induce an immune response.

The “intracellular signaling domain” means any oligopeptide or polypeptide domain known to function to transmit a signal causing activation or inhibition of a biological process in a cell, for example, activation of an immune cell such as a T cell or a NK cell. Examples include ILR chain, CD28, and/or CD3E.

As used herein, “cancer antigen” refers to (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, “CG oligodeoxynucleotides (CG ODNs)”, also referred to as “CpG ODNs”, are short single-stranded synthetic DNA molecules that contain a cytosine nucleotide (C) followed by a guanine nucleotide (G). In certain embodiments, the immunostimulatory oligonucleotide is a CG ODN.

As used herein the term “co-stimulatory ligand” includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-I (CD80), B7-2 (CD86), PD-L1, PD-L2, 4-1 BBL, OX40L, inducible costimulatory ligand (ICOS-L), intercellular adhesion molecule (rCAM), CD30L, CD40, CD70, CD83, HLA-G, MICA, MICE, HVEM, lymphotoxin beta receptor, TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-11BB, OX40, CD30, CD40, PD-I, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to, an MHC class I molecule, BTLA, and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A polypeptide or amino acid sequence “derived from” a designated polypeptide or protein or a “polypeptide fragment” refers to the origin of the polypeptide. Preferably, the polypeptide or amino acid sequence which is derived or is a fragment of is from a particular sequence that has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from or that are fragments of another peptide may have one or more mutations relative to the starting polypeptide, e.g., one or more amino acid residues which have been substituted with another amino acid residue or which has one or more amino acid residue insertions or deletions.

As used herein, the term “drug metabolite” refers to a therapeutic drug molecule or its intermediary or resulting products formed through the break down of the drug molecule that is capable of binding to major histocompatibility complex class I-related protein.

A polypeptide can comprise an amino acid sequence which is not naturally occurring. Such variants necessarily have less than 100% sequence identity or similarity with the starting molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

As used herein, the term antigen “cross-presentation” refers to presentation of exogenous protein antigens to T cells via MHC class I and class II molecules on APCs.

As used herein, the term “cytotoxic T lymphocyte (CTL) response” refers to an immune response induced by cytotoxic T cells. CTL responses are mediated primarily by CD8+ T cells.

As used herein, the term “effective dose” or “effective dosage” is defined as an amount sufficient to achieve or at least partially achieve the desired effect.

The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

As used herein, the term “effector cell” or “effector immune cell” refers to a cell involved in an immune response, e.g., in the promotion of an immune effector response. In some embodiments, immune effector cells specifically recognize an antigen. Examples of immune effector cells include, but are not limited to, Natural Killer (NK) cells, B cells, monocytes, macrophages, T cells (e.g., cytotoxic T lymphocytes (CTLs). In some embodiments, the effector cell is a T cell.

As used herein, the term “immune effector function” or “immune effector response” refers to a function or response of an immune effector cell that promotes an immune response to a target.

As used herein, “immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes). In particular embodiments, the immune cell is T cell.

As used herein, “immune response” refers to a response made by the immune system of an organism to a substance, which includes but is not limited to foreign or self proteins. Three general types of “immune response” include mucosal, humoral, and cellular immune responses. For example, the immune response can include the activation, expansion, and/or increased proliferation of an immune cell. An immune response may also include at least one of the following: cytokine production, T cell activation and/or proliferation, granzyme or perforin production, activation of antigen presenting cells or dendritic cells, antibody production, inflammation, developing immunity, developing hypersensitivity to an antigen, the response of antigen-specific lymphocytes to antigen, clearance of an infectious agent, and transplant or graft rejection.

As used herein, an “immunostimulatory oligonucleotide” is an oligonucleotide that can stimulate (e.g., induce or enhance) an immune response.

The terms “inducing an immune response” and “enhancing an immune response” are used interchangeably and refer to the stimulation of an immune response (i.e., either passive or adaptive) to a particular antigen.

The term “induce” as used with respect to inducing complement dependent cytotoxicity (CDC) or antibody-dependent cellular cytotoxicity (ADCC) refer to the stimulation of particular direct cell killing mechanisms.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a composition comprising an amphiphilic ligand conjugate).

The term “in vivo” refers to processes that occur in a living organism.

The term “in vitro” refers to processes that occur outside a living organism, such as in a test tube, flask, or culture plate.

As used herein, the term “ligand for a mucosal-associated invariant T-cell (MAIT)” or “MAIT ligand” refers any natural or synthetic molecule (e.g., small molecule, protein, peptide, lipid, carbohydrate, nucleic acid) or part or fragment thereof that can specifically bind to the MAIT.

As used herein, the terms “linked,” “operably linked,” “fused,” or “fusion,” are used interchangeably. These terms refer to the joining together of two more elements or components or domains, by an appropriate means including chemical conjugation or recombinant DNA technology. Methods of chemical conjugation (e.g., using heterobifunctional crosslinking agents) are known in the art as are methods of recombinant DNA technology.

The term “lipid” refers to a biomolecule that is soluble in nonpolar solvents and insoluble in water. Lipids are often described as hydrophobic or amphiphilic molecules which allows them to form structures such as vesicles or membranes in aqueous environments. Lipids include fatty acids, glycerolipids, glycerophospholipids, sphingolipids, sterol lipids (including cholesterol), prenol lipids, saccharolipids, and polyketides. In some embodiments, the lipid suitable for the amphiphilic ligand conjugates of the disclosure binds to human serum albumin under physiological conditions. In some embodiments, the lipid suitable for the amphiphilic ligand conjugates of the disclosure inserts into a cell membrane under physiological conditions. In some embodiments, the lipid binds albumin and inserts into a cell membrane under physiological conditions. In some embodiments, the lipid is a diacyl lipid. In some embodiments, the diacyl lipid includes at least 12 carbons. In some embodiments, the diacyl lipid includes 12-30 hydrocarbon units, 14-25 hydrocarbon units, or 16-20 hydrocarbon units. In some embodiments, the diacyl lipid includes 12, 13, 14, 15, 16, 17, 18, 19, 20, 21, 22, 23, 24, 25, 26, 27, 28, 29 or 30 carbons.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mal. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. In some embodiments, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “physiological conditions” refers to the in vivo condition of a subject. In some embodiments, physiological condition refers to a neutral pH (e.g., pH between 6-8).

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “riboflavin metabolite” refers to the intermediary or resulting products of riboflavin metabolism which include a ribityl group and are capable of binding to major histocompatibility complex class I-related protein.

As used herein, a “small molecule” is a molecule with a molecular weight below about 500 Daltons.

As used herein, a “small metabolite ligand” refers to a small molecule that is capable of binding to a major histocompatibility complex class-I related protein and is produced or used during all the physical and chemical processes within the body that create and use energy and include, but are not limited to, lipids, steroids, amino acids, organic acids, bile acids, eicosanoids, peptides, trace elements, and pharmacophore and drug breakdown products

As used herein, the term “subject” or “mammal” or “patient” includes any human or non-human animal. For example, the methods and compositions of the present invention can be used to treat a subject with a cancer or infection. The term “non-human animal” includes all vertebrates, e.g., mammals and non-mammals, such as non-human primates, sheep, dogs, cats, mice, horses, pigs, cows, chickens, amphibians, reptiles, etc.

The term “sufficient amount” or “amount sufficient to” means an amount sufficient to produce a desired effect, e.g., an amount sufficient to reduce the diameter of a tumor.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}, T_{H}2, T_{H}3, T_{H}17, T_{H}9, and T_{FH} cells, cytotoxic T cells (i.e., Tc cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. Treg cells or suppressor T cells) and subtypes, including CD4⁺FOXP3⁺ T_{reg} cells, CD4⁺FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. Any one or more of the aforementioned or unmentioned T cells may be the target cell type for a method of use of the invention.

As used herein, the term “T cell activation” or “activation of T cells” refers to a cellular process in which mature T cells, which express antigen-specific T cell receptors on their surfaces, recognize their cognate antigens and respond by entering the cell cycle, secreting cytokines or lytic enzymes, and initiating or becoming competent to perform cell-based effector functions. T cell activation requires at least two signals to become fully activated. The first occurs after engagement of the T cell antigen-specific receptor (TCR) by the antigen-major histocompatibility complex (MHC), and the second by subsequent engagement of co-stimulatory molecules (e.g., CD28). These signals are transmitted to the nucleus and result in clonal expansion of T cells, upregulation of activation markers on the cell surface, differentiation into effector cells, induction of cytotoxicity or cytokine secretion, induction of apoptosis, or a combination thereof.

As used herein, the term “T cell-mediated response” refers to any response mediated by T cells, including, but not limited to, effector T cells (e.g., CD8⁺ cells) and helper T cells (e.g., CD4⁺ cells). T cell mediated responses include, for example, T cell cytotoxicity and proliferation.

The term “T cell cytotoxicity” includes any immune response that is mediated by CDS⁺ T cell activation. Exemplary immune responses include cytokine production, CD8⁺ T cell proliferation, granzyme or perforin production, and clearance of an infectious agent.

As used herein, the term “target-binding domain” of an extracellular domain refers to a polypeptide found on the outside of the cell that is sufficient to facilitate binding to a target. The target-binding domain will specifically bind to its binding partner, i.e., the target. As non-limiting examples, the target-binding domain can include an antigen-binding domain of an antibody, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway. In one embodiment, the ligand can be non-native to the genome. Optionally, the ligand has a conserved function across at least two species. In some embodiments, the ligand is a cancer antigen. In some embodiments, the ligand is a tumor-associated antigen.

A “therapeutic antibody” is an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fv fragments of antibodies, minibodies and diabodies. Any of the therapeutic antibodies known in the art to be useful for cancer therapy can be used in combination therapy with the compositions described herein. Therapeutic antibodies may be monoclonal antibodies or polyclonal antibodies. In preferred embodiments, the therapeutic antibodies target cancer antigens.

As used herein, “therapeutic protein” refers to any polypeptide, protein, protein variant, fusion protein and/or fragment thereof which may be administered to a subject as a medicament. The term “therapeutically effective amount” is an amount that is effective to ameliorate a symptom of a disease. A therapeutically effective amount can be a “prophylactically effective amount” as prophylaxis can be considered therapy.

The terms “treat,” “treating,” and “treatment,” as used herein, refer to therapeutic or preventative measures described herein. The methods of “treatment” employ administration to a subject, in need of such treatment, an amphiphilic ligand conjugate of the present disclosure, for example, a subject receiving T cell immunotherapy. In some embodiments, an amphiphilic ligand conjugate is administered to a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “tumor-associated antigen” refers to an antigen that is produced in a tumor and can be detected by the immune system to trigger an immune response. Tumor-associated antigens have been identified in many human cancers including lung, skin, hematologic, brain, liver, breast, rectal, bladder, and stomach cancers.

As used herein, “vaccine” refers to a formulation which contains an amphiphilic ligand conjugate and a TCR modified immune cell as described herein, optionally combined with an adjuvant, which is in a form that is capable of being administered to a vertebrate and which induces a protective immune response sufficient to induce immunity to prevent and/or ameliorate a disease or condition (e.g., cancer) and/or to reduce at least one symptom of a disease or condition (e.g., cancer) and/or to enhance the efficacy of a TCR modified immune cell, e.g., a TCR modified T cell. Typically, the vaccine comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the inducing a protective immune response to induce immunity to prevent and/or ameliorate a disease or condition (e.g., cancer) and/or to reduce at least one symptom of a disease or condition and/or to enhance the efficacy of a TCR modified immune cells, e.g., a TCR modified T cell.

### DETAILED DESCRIPTION

Described herein are methods for stimulating an immune response to a target cell population in a subject, where the methods include administering to the subject an amphiphilic lipid conjugate including a lipid, a peptide (e.g., a tumor associated antigen), and optionally a linker, and an immune cell modified with a T cell receptor (TCR) e.g., a TCR modified T cell, where the T cell receptor binds the peptide of the amphiphilic ligand conjugate. Such methods are useful for, e.g., treating a human subject with cancer.

### Amphiphilic Conjugates

In certain embodiments, amphiphilic conjugates are used with a T cell receptor expressing immune cell therapy. In some embodiments, the amphiphilic conjugate stimulates a specific immune response against a specific target, such as a tumor-associated antigen. In some embodiments, the amphiphilic conjugate induces activation, expansion, or proliferation of an immune cell expressing a T cell receptor in vivo. In some embodiments, the amphiphilic conjugate induces activation, proliferation, phenotypic maturation, or acquisition of cytotoxic function of a TCR T cell in vitro by culturing the TCR T cell in the presence of a dendritic cell including an amphiphilic ligand conjugate. In some embodiments, the amphiphilic conjugate includes a ligand, and is referred to herein as an “amphiphilic ligand conjugate.”

The structure of an amphiphilic ligand conjugate as described herein includes a lipophilic moiety, or “lipid tail”, (e.g., DSPE) covalently linked, optionally via a linker (e.g., PEG-2000), to one or more cargos. The amphiphilic ligand conjugate cargo can include a T cell receptor target. The modularity of this design allows for various ligands including, but not limited to, small molecules (e.g., fluorescein isothiocyanate (FITC)), short peptides (e.g., a linear peptide providing an epitope specific for a T cell receptor), ligands for MAIT cells (e.g. riboflavin metabolites and drug metabolites), or modular protein domains (e.g., folded polypeptide or polypeptide fragment providing a conformational epitope specific for a T cell receptor), or any one of the T cell receptor targets described herein, to be covalently linked to the lipid, resulting in amphiphilic ligand conjugates with tailored specificity.

Upon administration, without being bound by theory, the amphiphilic ligand conjugate is thought to be delivered to lymph nodes where the lipid tail portion is inserted into the membrane of antigen presenting cells (APCs), resulting in the decoration of the APC with ligands. The embedded ligands function as specific targets for an engineered receptor (i.e., a T cell receptor) expressed on the surface of prior, subsequent, or co-administered immune cells expressing said receptor, resulting in the recruitment of the immune cells to the ligand-decorated APCs. Interaction of the engineered receptor with the embedded ligand provides a stimulatory signal through the engineered receptor while the APC additionally presents other naturally occurring co-stimulatory signals, resulting in optimal immune cell activation, prolonged survival, and efficient memory formation.

Amphiphilic ligand conjugates can be generated using methods known in the art, such as those described in US 2013/0295129, which is hereby incorporated by reference in its entirety. For example, N-terminal cysteine modified peptides can be dissolved in DMF (dimethylformamide) and mixed with 2 equivalents Maleimide-PEG₂₀₀₀-DSPE (Laysan Bio, Inc.), and agitating the mixture at room temperature for 24 hours. Bioconjugation can be assessed by HPLC analysis.

### Lipid Conjugates

In certain embodiments, a lipid conjugate (e.g., an amphiphilic ligand conjugate), as described in US 2013/0295129, herein incorporated by reference in its entirety, is used in the methods disclosed herein. A lipid conjugate includes an albumin-binding lipid and a cargo to efficiently target the cargo to lymph nodes in vivo. Lipid conjugates bind to endogenous albumin, which targets them to lymphatics and draining lymph nodes where they accumulate due to the filtering of albumin by antigen presenting cells. In some embodiments, the lipid conjugate includes an antigenic peptide, molecular adjuvant, or ligand for a MAIT cell, and thereby induces or enhances a robust immune response. In some embodiments, the lipid conjugate includes a T cell receptor ligand, and thereby can induce or enhance expansion, proliferation, and/or activation of immune cells expressing a T cell receptor.

Lymph node-targeting conjugates typically include three domains: a highly lipophilic, albumin-binding domain (e.g., an albumin-binding lipid), a cargo such as a peptide, ligand for a MAIT cell, or molecular adjuvant, and a polar block linker, which promotes solubility of the conjugate and reduces the ability of the lipid to insert into cellular plasma membranes. Accordingly, in certain embodiments, the general structure of the conjugate is L-P-C, where “L” is an albumin-binding lipid, “P” is a polar block, and “C” is a cargo such as a peptide or a molecular adjuvant. In some embodiments, the cargo itself can also serve as the polar block domain, and a separate polar block domain is not required. Therefore, in certain embodiments the conjugate has only two domains: an albumin-binding lipid and a cargo, e.g., a peptide.

In some embodiments, the cargo of the conjugate is a peptide or a ligand for a MAIT cell, such as in an amphiphilic ligand conjugate. In other embodiments, is the peptide is an antigenic peptide. In further embodiments, the peptide is a tumor associated antigenic peptide. In yet other embodiments, the peptide is a T cell receptor target, e.g., an epitope. In some embodiments, the amphiphilic ligand conjugate is administered or formulated with an adjuvant, wherein the adjuvant is an amphiphilic conjugate including a molecular adjuvant such as an immunostimulatory oligonucleotide.

Optionally, the amphiphilic ligand conjugate is in the form of a pharmaceutically acceptable salt. The term “pharmaceutically acceptable salt,” as used herein, means any pharmaceutically acceptable salt of a conjugate, oligonucleotide, or peptide disclosed herein. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. References to conjugates (e.g., amphiphilic ligand conjugates), oligonucleotides, or peptides in the claims are to be interpreted to optionally include pharmaceutically acceptable salts thereof.

### Lipids

The amphiphilic ligand conjugates typically include a hydrophobic lipid. The lipid can be linear, branched, or cyclic. In certain embodiments, the activity relies, in part, on the ability of the conjugate to insert itself into a cell membrane. Therefore, lymph node-targeted conjugates typically include a lipid that undergo membrane insertion under physiological conditions. Lipids suitable for membrane insertion can be selected based on the ability of the lipid or a lipid conjugate including the lipid to bind to interact with a cell membrane. Suitable methods for testing the membrane insertion of the lipid or lipid conjugate are known in the art.

Examples of preferred lipids for use in lymph node targeting lipid conjugates include, but are not limited to, fatty acids with aliphatic tails of 3-30 carbons including, but not limited to, linear unsaturated and saturated fatty acids, branched saturated and unsaturated fatty acids, and fatty acids derivatives, such as fatty acid esters, fatty acid amides, and fatty acid thioesters, diacyl lipids, cholesterol, cholesterol derivatives, and steroid acids such as bile acids, Lipid A or combinations thereof.

In certain embodiments, the lipid is a diacyl lipid or two-tailed lipid. In some embodiments, the tails in the diacyl lipid contain from about 12 to about 30 carbons (e.g., 13 14, 15, 16, 17, 18, 19, 20, 21, 22, 23, 24, 25, 26, 27, 28, or 29). In some embodiments the tails in the diacyl lipid contain about 14 to about 25 carbons (e.g., 15, 16, 17, 18, 19, 20, 21, 22, 23, or 24). In some embodiments, the tails of the diacyl lipid contain from about 16 to about 20 carbons (e.g., 17, 18, or 19). In some embodiments, the diacyl lipid comprises 12, 13, 14, 15, 16, 17, 18, 19, 20, 21, 22, 23, 24, 25, 26, 27, 28, 29 or 30 carbons.

The carbon tails of the diacyl lipid can be saturated, unsaturated, or combinations thereof. The tails can be coupled to the head group via ester bond linkages, amide bond linkages, thioester bond linkages, or combinations thereof. In a particular embodiment, the diacyl lipids are phosphate lipids, glycolipids, sphingolipids, or combinations thereof.

Preferably, membrane-inserting conjugates include a lipid that is μ or fewer carbon units in length, as it is believed that increasing the number of lipid units can reduce insertion of the lipid into plasma membrane of cells, allowing the lipid conjugate to remain free to bind albumin and traffic to the lymph node.

### Molecular Adjuvants

In certain embodiments, amphiphilic oligonucleotide conjugates are used with the amphiphilic ligand conjugate. The oligonucleotide conjugates typically contain an immunostimulatory oligonucleotide.

In certain embodiments, the immunostimulatory oligonucleotide can serve as a ligand for pattern recognition receptors (PRRs). Examples of PRRs include the Toll-like family of signaling molecules that play a role in the initiation of innate immune responses and also influence the later and more antigen specific adaptive immune responses. Therefore, the oligonucleotide can serve as a ligand for a Toll-like family signaling molecule, such as Toll-Like Receptor 9 (TLR9).

For example, unmethylated CpG sites can be detected by TLR9 on plasmacytoid dendritic cells and B cells in humans (Zaida, et al., Infection and Immunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, as discussed in more detail below, some oligonucleotides including CG can have a modified backbone, for example a phosphorothioate (PS) backbone.

In certain embodiments, an immunostimulatory oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced drug delivery reviews 61(3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J of Immun. 164(3):1617-2 (2000), each of which is incorporated herein by reference).

According to some embodiments, a lipophilic-CpG oligonucleotide conjugate is used to enhance an immune response to an antigen. An exemplary lipophilic-CpG oligonucleotide conjugate includes the sequence 5′-TCGTCGTTTTGTCGTTTTGTCGTT-3′ (SEQ ID NO:1125). The CpG oligonucleotide sequence is linked, at its 5′ end, to a lipid, such as the following:

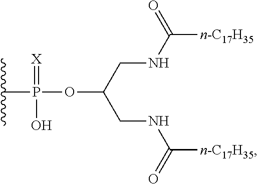

where X is O or S. Preferably, X is S. The CpG oligonucleotide may be directly bonded to the lipid. Alternatively, the CpG oligonucleotide may be linked to the lipid through a linker, such as GG. In the exemplary CpG oligonucleotide, all internucleoside groups are phosphorothioates (e.g., all internucleoside groups in the compound may be phosphorothioates).

Another exemplary lipophilic-CpG oligonucleotide is represented by the following, wherein “L” is a lipophilic compound, such as diacyl lipid, “Gₙ” is a guanine repeat linker and “n” represents 1, 2, 3, 4, or 5.
5′-L-GₙTCCATGACGTTCCTGACGTT-3′ (SEQ ID NO: 1124)

Other PRR Toll-like receptors include TLR3, and TLR7 which may recognize double-stranded RNA, single-stranded and short double-stranded RNAs, respectively, and retinoic acid-inducible gene I (RIG-I)-like receptors, namely RIG-1 and melanoma differentiation-associated gene 5 (MDA5), which are best known as RNA-sensing receptors in the cytosol. Therefore, in certain embodiments, the oligonucleotide contains a functional ligand for TLR3, TLR7, or RIG-1-like receptors, or combinations thereof.

Examples of immunostimulatory oligonucleotides, and methods of making them are known in the art, see for example, Bodera, P. Recent Pat Inflamm Allergy Drug Discov. 5(1):87-93 (2011), incorporated herein by reference.

In certain embodiments, the oligonucleotide cargo includes two or more immunostimulatory sequences.

The oligonucleotide can be between 2-100 nucleotide bases in length, including for example, 5 nucleotide bases in length, 10 nucleotide bases in length, 15 nucleotide bases in length, 20 nucleotide bases in length, 25 nucleotide bases in length, 30 nucleotide bases in length, 35 nucleotide bases in length, 40 nucleotide bases in length, 45 nucleotide bases in length, 50 nucleotide bases in length, 60 nucleotide bases in length, 70 nucleotide bases in length, 80 nucleotide bases in length, 90 nucleotide bases in length, 95 nucleotide bases in length, 98 nucleotide bases in length, 100 nucleotide bases in length or more.

The 3′ end or the 5′ end of the oligonucleotides can be conjugated to the polar block or the lipid. In certain embodiments the 5′ end of the oligonucleotide is linked to the polar block or the lipid.

The oligonucleotides can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds. In certain embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In certain embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In certain embodiments, the analogs have a substantially uncharged, phosphorus containing backbone.

### Mucosal-Associated Invariant T-cell (MAIT) Ligands

In some embodiments, the amphiphilic ligand conjugate cargo is a ligand for a MAIT cell. The ligand for a MAIT cell is capable of binding to a major histocompatibility complex class I-related protein.

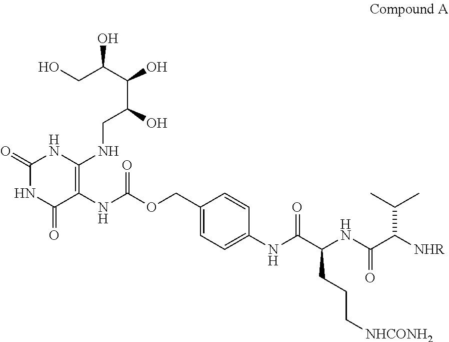

In some embodiments, the ligand for a MAIT cell is a small molecule metabolite. In some embodiments, the ligand for a MAIT cell is a vitamin B (e.g., riboflavin) metabolite. For example, the vitamin B metabolite may be 5-(2-oxopropylideneamino)-6-d-ribitylaminouracil (5-OP-RU), 5-(2-oxoethylideneamino)-6-D-ribitylaminouracil (5-OE-RU), 6,7-dimethyl-8-D-ribityllumazine (RL-6,7-diMe), 7-hydroxy-6-methyl-8-D-ribityllumazine (RL-6-Me-7-OH), 6-hydroxymethyl-8-D-ribityl-lumazine, 6-(1H-indol-3-yl)-7-hydroxy-8-ribityllumazine, 6-(2-carboxyethyl)-7-hydroxy8-ribityllumazine. In some embodiments, ligand for a MAIT cell is valine-citrulline-p-aminobenzyl carbamate modified ligand. In some embodiments, the MAIT ligand may be a 5-amino-6-D-ribityl aminouracil (5-A-RU) prodrug. For example, the MAIT ligand may be a valine-citrulline-p-aminobenzyl carbamate modified 5-A-RU. In some embodiments, the MAIT ligand has the structure of Compound A, wherein R is a fluorenylmethyloxycarbonyl protecting group.

In some embodiments, the ligand for a MAIT cell is a drug metabolite. For example, the drug metabolite may be benzobromarone, chloroxine, diclofenac, 5-hydroxy diclofenac, 4-hydroxy diclofenac, floxuridine, galangin, menadione sodium bisulfate, mercaptopurine, tetrahydroxy-1,4-quinone hydrate.

### Amphiphilic Ligand Conjugate Cargos

In some embodiments, the amphiphilic ligand conjugate cargo is an antigenic protein, polypeptide, peptide, or epitope, such as a tumor-associated antigen or portion thereof (e.g., an epitope). In some embodiments, the amphiphilic ligand conjugate binds to a T cell receptor. Accordingly, the methods and compositions described herein utilize an amphiphilic ligand conjugate that binds to a T cell receptor expressing cell.

The peptide can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids. In some embodiments, a peptide can be greater than 50 amino acids. In some embodiments, the peptide can be >100 amino acids.

A protein/peptide can be linear, branched, or cyclic. The peptide can include D amino acids, L amino acids, or a combination thereof. The peptide or protein can be conjugated to the polar block or lipid at the N-terminus or the C-terminus of the peptide or protein.

The protein or polypeptide can be any protein or peptide that can induce or increase the ability of the immune system to develop antibodies and T cell responses to the protein or peptide.

A T cell receptor ligand of an amphiphilic ligand conjugate described herein can be a tumor associated antigen.

### Cancer and Tumor-Associated Antigens

In some embodiments, the amphiphilic ligand conjugate described herein includes a protein, polypeptide or peptide. The protein, polypeptide or peptide may include a post-translational modification, for example, glycosylation, ubiquitination, phosphorylation, nitrosylation, methylation, acetylation, amidation, hydroxylation, sulfation, or lipidation. The peptide may be between 3 amino acids and 50 amino acids in length. For example, the peptide may be 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In some embodiments, the protein, polypeptide, or peptide is an antigen. For example, the antigen may be a cancer antigen e.g., a tumor-associated antigen. In some embodiments, the antigen is an antigen that has been identified to play a role in cancer. In particular embodiments, the antigen has a polypeptide sequence having at least 85% sequence identity to the sequence of any one of SEQ ID NOs: 1-97 or 1125-1183. In some embodiments, the antigen includes the sequence any one of SEQ ID NOs: 1-97 or 1125-1183, or includes Ganglioside G2 or Ganglioside G3. In other embodiments, the antigen consists of the sequence of any one of SEQ ID NOs: 1-97 or 1125-1183.

A cancer antigen is an antigen that is typically expressed preferentially by cancer cells (i.e., it is expressed at higher levels by cancer cells than by non-cancer cells) and in some instances it is expressed solely by cancer cells. In some embodiments, the cancer antigen is a tumor-associated antigen. The cancer antigen may be expressed within a cancer cell or on the surface of the cancer cell. The cancer antigen can be, but is not limited to, any one of Human papillomavirus (HPV) E6 protein (e.g., HPV-6 E6 protein (SEQ ID NO: 1172), HPV-11 E6 protein (SEQ ID NO: 1171), HPV-16 E6 protein (SEQ ID NO: 1169), or an HPV-18 E6 protein (SEQ ID NO: 1170)), HPV E7 protein (e.g., HPV-6 E7 protein (SEQ ID NO: 1173), HPV-11 E7 protein (SEQ ID NO: 1174), HPV-16 E7 protein (SEQ ID NO: 1175), or an HPV-18 E7 protein (SEQ ID NO: 1176)), Kirsten rat sarcoma (mKRAS) (SEQ ID NO: 1177) (e.g., G12A, G12C, G12D, G12E, G12F, G12H, G121, G12K, G12L, G12M, G12N, G12P, G12Q, G12R, G12S, G12T, G12V, G12W, G12Y, G13C, G13D, Q61A, Q61C, Q61E, Q61F, Q61G, Q61H, Q611, Q61K, Q61L, Q61M, Q61N, Q61N, Q61P, Q61R, Q61T, Q61V, and Q61W variants), Wilms tumor 1 (WT-1) (SEQ ID NO: 19), New York Esophageal Squamous Cell Carcinoma (NYESO) (SEQ ID NO: 9), Mucin 1 (MUC1) (SEQ ID NO: 67), Epidermal growth factor receptor (EGFR) (SEQ ID NO: 1125), Epidermal growth factor receptor variant III (EGFRviii), Phosphoinositide 3-kinase (PI3K) (SEQ ID NO: 1126), Latent membrane protein 2 (LMP2) (SEQ ID NO: 17), Receptor tyrosine-protein kinase erbB-2 (HER-2/neu) SEQ ID NO: 70), Melanoma antigen A3 (MAGE A3) (SEQ ID NO: 12), p53 wild-type (SEQ ID NOS: 86 and 89), p53 mutant, Prostate-specific membrane antigen (PSMA) (SEQ ID NO: 1127), Ganglioside G2 (GD2) (PubChem CID 6450346), Ganglioside G3 (GD3) (PubChem CID 20057323), Carcinoembryonic antigen (CEA) (SEQ ID NO: 1128), Melanoma antigen recognized by T cells (MelanA/MART-1) (SEQ ID NO: 8), Glycoprotein 100 (gp100) (SEQ ID NO: 1129), Proteinase3 (SEQ ID NO: 1130), Breakpoint cluster region protein-Tyrosine protein kinase (bcr-abl) (SEQ ID NO: 1131), Tyrosinase (SEQ ID NOS: 11 and 25), Survivin (SEQ ID NO: 1132), Prostate-specific antigen (PSA) (SEQ ID NO: 1133), human Telomerase reverse transcriptase (hTERT) (SEQ ID NO: 1134), Ephrin type-A receptor 2 (EphA2) (SEQ ID NO: 1135), Pancreatitis associated protein (PAP) (SEQ ID NO:1136), Mucolipidaryl hydrocarbon receptor-interacting protein (ML-AIP) (SEQ ID NO: 1178), Alpha fetoprotein (AFP) (SEQ ID NO: 1137), Epithelial cell adhesion molecule (EpCAM) (SEQ ID NO: 1138), ETS-related gene (ERG) (SEQ ID NO: 1139) (e.g., TMOPRSS2 ETS fusion), NA17 (SEQ ID NO: 1140), Paired Box 3 (PAX3) (SEQ ID NO: 1141), Anaplastic lymphoma kinase (ALK) (SEQ ID NO: 1142), androgen receptor (SEQ ID NO: 1143), Cyclin B (SEQ ID NO: 1144), N-myc proto-oncogene protein (MYCN) (SEQ ID NO: 1145), Rho protein coding (RhoC) (SEQ ID N: 1146), Tyrosinase-related protein-2 (TRP-2) (SEQ ID NO: 1147), Mesothelin (SEQ ID NO: 1148), Prostate stem cell antigen (PSCA) (SEQ ID NO: 1149), Melanoma antigen A1 (MAGE A1) (SEQ ID NO: 15), Cytochrome P450 Family 1 Subfamily B Member 1 (CYP1 B1) (SEQ ID NO: 1150), Placenta-specific protein 1 precursor (PLAC1) (SEQ ID NO: 1151), Monosialodihexosylganglioside (GM3), Brother of regulator of imprinted sites (BORIS) (SEQ ID NO: 1152), Tenascin (Tn) (SEQ ID NO: 1153), Globohexasylceraminde (GloboH), Translocation-Ets-leukemia virus protein-6—acute myeloid leukemia 1 protein (ETV6-AML) (SEQ ID NO: 1154), NY breast cancer antigen 1 (NY-BR-1) (SEQ ID NO: 1179), Regulator of G protein signaling 5 (RGS5) (SEQ ID NO: 1155), Squamous cell carcinoma antigen recognized by T cells 3 (SART3) (SEQ ID NO: 1156), Salmonella enterotoxin (STn) (SEQ ID NO: 1157), Carbonic Anhydrase IX (SEQ ID NO: 42), Paired box gene 5 (PAXS) (SEQ ID NO: 1158), Cancer testis antigen (OY-TES1) (SEQ ID NO: 1159), Tyrosine-protein kinase Lck (LCK) (SEQ ID NO: 1160), human high molecular weight-melanoma associated antigen (HMWMAA) (SEQ ID NO: 1180), A-kinase anchoring protein 4 (AKAP-4) (SEQ ID NO: 1161), Protein SSX2 (SSX2) (SEQ ID NO: 88), X-antigen family member 1 (XAGE-1) (SEQ ID NO: 1162), B7 homolog 3 (B7H3) (SEQ ID NO: 1163), Legumain (SEQ ID NO: 1164), Tyrosine-protein kinase receptor (Tie 2) (SEQ ID NO: 1165), P antigen family member 4 (Page4) (SEQ ID NO: 1166), Vascular endothelial growth factor receptor 2 (VEGFR2) (SEQ ID NO: 1167), Melanoma-cancer testis antigen 1 (MAD-CT-1) (SEQ ID NO: 1182), Fibroblast activation protein (FAP) (SEQ ID NO: 1181), Platelet derived growth factor receptor beta (PDGFR-B) (SEQ ID NO: 1168), Melanoma-cancer testis antigen 2 (MAD-CT-2) (SEQ ID NO: 1183).

For example, in lung cancer, several antigens have been identified including Matrix protein 1 from Influenza A virus (SEQ ID NO: 1), Epstein-Barr nuclear antigen 3 from Human herpesvirus 4 (SEQ ID NO: 2), Matrix protein from Human respiratory syncytial virus B1 (SEQ ID NO: 3), Phospholipid-transporting ATPase ABCA1 (SEQ ID NO: 4), Signal-regulatory protein delta (SEQ ID NO: 5), Mini-chromosome maintenance complex-binding protein (SEQ ID NO: 6), and Fragile X mental retardation 1 neighbor protein (SEQ ID NO: 7).

In skin cancer, several antigens have been identified including Melanoma antigen recognized by T cells 1 (MART-1) (SEQ ID NO: 8), Autoimmunogenic cancer/testis antigen NY-ESO-1 (LAGE-2) (SEQ ID NO:9), Melanocyte protein PMEL (ME20-M) (SEQ ID NO: 10), Tyrosinase (SK29-AB) (SEQ ID NO: 11), Melanoma-associated antigen 3 (MAGE-3) (SEQ ID NO: 12), Cyclin-dependent kinase 4 (PSK-J3) (SEQ ID NO: 13), Thymosin beta-10 (SEQ ID NO: 14), Melanoma-associated antigen 1 (MAGE-1) (SEQ ID NO: 15).

In hematologic cancers, several antigens have been identified including Protein Tax-1 (Protein X-LOR) (SEQ ID NO: 16), Latent membrane protein 2 from Human herpesvirus 4 (SEQ ID NO: 17), Myeloblastin (SEQ ID NO: 18), Wilms tumor protein (SEQ ID NO: 19), 65 kDa phosphoprotein (pp65) (SEQ ID NO: 20), Latent membrane protein 1 from Human herpesvirus 4 (LMP-1) (SEQ ID NO: 21), Epstein-Barr nuclear antigen 3 from Human herpesvirus 4 (EBNA-3) (SEQ ID NO: 22), Epstein-Barr nuclear antigen 1 from Human herpesvirus 4 (EBNA-1) (SEQ ID NO: 23), RNA helicase (SEQ ID NO: 24), Tyrosinase (LB24-AB) (SEQ ID NO: 25), Envelope glycoprotein gp62 (gp46) (SEQ ID NO: 26), Rho GTPase-activating protein 45 (SEQ ID NO: 27), Unconventional myosin-Ig (SEQ ID NO: 28), Transferrin receptor protein 1 (TfR1) (SEQ ID NO: 29), ETS translocation variant 5 (SEQ ID NO: 30), E3 ubiquitin-protein ligase Mdm2 (SEQ ID NO: 31), U1 small nuclear ribonucleoprotein 70 kDa (snRNP70) (SEQ ID NO: 32), Cell division cycle-associated 7-like protein (Protein JPO) (SEQ ID NO: 33), Serine/threonine-protein kinase pim-1 (SEQ ID NO: 34), Death-associated protein kinase 2 (DAP kinase 2) (SEQ ID NO: 35), HTLV-1 basic zipper factor (HBZ) (SEQ ID NO: 36), RNA polymerase II subunit A C-terminal domain phosphatase (SEQ ID NO: 37), B-lymphocyte surface antigen B4 (SEQ ID NO: 38), Hyaluronan mediated motility receptor (SEQ ID NOL: 39), Perilipin-2 (SEQ ID NO: 40), Preferentially expressed antigen of melanoma (SEQ ID NO: 41), Carbonic anhydrase (SEQ ID NO: 42), Ras-specific guanine nucleotide-releasing factor 1 (Ras-GRF1) (SEQ ID NO: 43), Tumor protein p53-inducible protein 11 (SEQ ID NO: 44), E3 ubiquitin-protein ligase Mdm2 (SEQ ID NO: 45), Vesicle-associated membrane protein 3 (SEQ ID NO: 46), Protein mono-ADP-ribosyltransferase PARP3 (SEQ ID NO: 47), ATP-binding cassette sub-family A member 6 (SEQ ID NO:48), B-lymphocyte antigen CD19 (SEQ ID NO: 49), Dynamin-binding protein (SEQ ID NO: 50), Pro-cathepsin H (SEQ ID NO: 51), Transferrin receptor protein 1 (TfR1) (SEQ ID NO: 52), Transmembrane emp24 domain-containing protein 4 (SEQ ID NO: 53), Fibromodulin (SEQ ID NO: 54), B-lymphocyte antigen CD20 (SEQ ID NO: 55), Zinc finger protein 216 (SEQ ID NO: 56), Integrator complex subunit 13 (SEQ ID NO: 57), Cadherin EGF LAG seven-pass G-type receptor 1 (hFmi2) (SEQ ID NO: 58), Melanoma antigen preferentially expressed in tumors (SEQ ID NO: 59), Dmx-like 1 (SEQ ID NO: 60), Baculoviral IAP repeat-containing protein 5 (SEQ ID NO: 61), Vesicle-associated membrane protein 3 (SEQ ID NO: 62), B-lymphocyte antigen CD19 (SEQ ID NO: 63), Interleukin-4 receptor subunit alpha (SEQ ID NO: 64), Cyclin-dependent kinase 4 (SEQ ID NO: 65), Circadian clock protein PASD1 (SEQ ID NO: 66), Mucin-1 (SEQ ID NO: 67), Inactive tyrosine-protein kinase transmembrane receptor ROR1 (SEQ ID NO: 68),

In brain cancer, the antigen Protein E7 from Human papillomavirus type 45 (SEQ ID NO: 69) has been identified.

In liver cancer, several antigens have been identified including Receptor tyrosine-protein kinase erbB-2 (SEQ ID NO: 70), Protein 13 (SEQ ID NO: 71), Intestinal protein OCI-5 (MXR7) (SEQ ID NO: 72), Chromodomain-helicase-DNA-binding protein 3 (SEQ ID NO: 73), RNA-directed RNA polymerase L (SEQ ID NO: 74), Programmed cell death protein 7 (SEQ ID NO: 75), and Interleukin-1 beta (SEQ ID NO: 76). In kidney cancer, several antigens have been identified including Protein enabled homolog (SEQ ID NO: 77), Myotubularin (SEQ ID NO: 78), Arachidonate-CoA ligase (VLCS-3) (SEQ ID NO: 79), Tyrosine-protein phosphatase non-receptor type 12 (SEQ ID NO: 80), Cytochrome c oxidase assembly factor 6 homolog (SEQ ID NO: 81), Methionine synthase reductase (SEQ ID NO: 82), Serine/threonine-protein kinase (SMG-1) (SEQ ID NO: 83), and EF-hand calcium-binding domain-containing protein 13 (SEQ ID NO: 84).

In breast cancer and thoracic cancer, several antigens have been identified including Mammaglobin-A (SEQ ID NO: 85), Cellular tumor antigen p53 (SEQ ID NO: 86), Receptor tyrosine-protein kinase erbB-2 (SEQ ID NO: 87), Protein SSX2 (SEQ ID NO: 88), Cellular tumor antigen p53 (SEQ ID NO: 89), and Heat shock 70 kDa protein 1 B (HSPA1 B) (SEQ ID NO: 90).

In cervical cancer, several antigens have been identified including Protein E7 from Alphapapillomavirus (SEQ ID NO: 91), Protein E6 from Alphapapillomavirus (SEQ ID NO: 92), Major capsid protein L1 from Human papillomavirus 16 (SEQ ID NO: 93), Replication protein E1 from Human papillomavirus 16 (SEQ ID NO: 94), Regulatory protein E2 from Human papillomavirus 16 (SEQ ID NO: 95), and Non-structural protein 2a from Human coronavirus OC43 (SEQ ID NO: 96).

In rectal cancer, the antigen Protein E7 from Alphapapillomavirus (SEQ ID NO: 91) has been identified.

In bladder cancer, the antigen C-terminal-binding protein 1 (SEQ ID NO: 97) has been identified.

In stomach cancer, the antigen the antigen Protein E7 from Alphapapillomavirus (SEQ ID NO: 91) has been identified.

The peptide included in the amphiphilic conjugate may be an epitope of an antigen. An antigen includes one or more epitopes that is recognized, targeted, or bound by a given antibody or T cell receptor. An epitope may be a linear epitope, for example, a contiguous sequence of nucleic acids or amino acids. An epitope may also be a conformational epitope, for example, an epitope that contains amino acids that form an epitope in the folded conformation of the protein. A conformational epitope may contain non-contiguous amino acids from a primary amino acid sequence. As another example, a conformational epitope may include nucleic acids that form an epitope in the folded conformation of an immunogenic sequence based on its secondary structure or tertiary structure. The epitope of the antigen may be a biologically active polypeptide fragment of the antigen, for example the epitope may be any length shorter than the antigen. In some embodiments, the epitope may include between 50 and 3 (e.g., 49, 48, 47, 46, 45, 44, 43, 42, 41, 42, 41, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, or 3) amino acid residues, between 25 and 5 (e.g., 24, 23, 22, 21, 20, 19, 18, 17, 16, 15, 14, 13, 12, 11, 10, 9, 8, 7, 6, or 5) amino acids residues, or between 15 and 6 (e.g., 14, 13, 12, 11, 10, 9, 8, 7, or 6) in length of the antigen.

In some embodiments, the epitope may be a fragment of the sequence of any one SEQ ID NOs: 1-97 or 1125-1183, or a fragment of Ganglioside G2 or Ganglioside G3. Further examples of epitopes that may be included in an amphiphilic lipid conjugate include any one of the epitopes described in Table 1. In some embodiments, the epitope included in the amphiphilic conjugate includes a polypeptide sequence having at least 85% (e.g., at least 86%, at least 87%, at least 88%, at least 89%, at least 90%, at least 95%, or 100%) sequence identity to the sequence of any one of SEQ ID NOs: 98-1123. In some embodiments, the peptide included in the amphiphilic conjugate may include any one of the epitopes described in Table 1, including any one of the sequences of SEQ ID NOs: 98-1123.

TABLE 1
Epitopes derived from tumor associated antigens

| SEQ | | | SEQ | | |
|---|---|---|---|---|---|
| ID | | | ID | | |
| NO: | Sequence | Origin | NO: | Sequence | Origin |
| 98 | GLPTDTIRKEFRTRM | 1-phosphatidylinositol 4,5- | 612 | ISEYRHYCY | Protein E6 |
| | | bisphosphate | | | |
| | | phosphodiesterase beta-4 | | | |
| | | isoform X10 [Homo | | | |
| | | sapiens] | | | |
| 99 | LFGLGKDEGWGPPAR | 5′-nucleotidase, cytosolic | 613 | KLPDLCTEL | protein E6 |
| | | IIIB | | | |
| 100 | TFNCHHARPWHNQF | 5-hydroxytryptamine | 614 | KLPQLCTEL | Protein E6 |
| | V | receptor 3D isoform 3 | | | |
| | | precursor [Homo sapiens] | | | |
| 101 | NLVPMVATV | 65 kDa lower matrix | 615 | TIHDIILECV | Protein E6 |
| | | phosphoprotein | | | |
| 102 | TPRVTGGGAM | 65 kDa lower matrix | 616 | VYDFAFRDL | Protein E6 |
| | | phosphoprotein | | | |
| 103 | FEGTEMWNPNRELSE | Acetylcholinesterase (Yt | 617 | DKKQRFHNIRG | Protein E6 |
| | | blood group) [Homo | | RWTGRCMSCC | |
| | | sapiens] | | RSSRTRRETQL | |
| 104 | AIGIGAYLV | Acetyl-CoA carboxylase 2 | 618 | FRDLCIVYRDG | Protein E6 |
| | | | | NPYAVCDKCLK | |
| | | | | FYSKISEYRHY | |
| 105 | SVQGIIIYR | ADP/ATP translocase 2 | 619 | KLPQLCTELQT | Protein E6 |
| | | | | TIHDIILECVYCK | |
| | | | | QQLLRREV | |
| 106 | VDGIGILTI | ADP-ribose | 620 | MHQKRTAMFQ | Protein E6 |
| | | pyrophosphatase | | DPQERPRKLPQ | |
| | | | | LCTELQTTIHDI | |
| 107 | SASVQRADTSL | AN1-type zinc finger | 621 | RCINCQKPLCP | Protein E6 |
| | | protein 5 | | EEKQRHLDKKQ | |
| | | | | RFHNIRGRWT | |
| 108 | ATIIDILTK | annexin I | 622 | EKQRHLDKKQR | Protein E6 |
| | | | | FHNI | |
| 109 | ILEGIGILAV | Anoctamin-3 | 623 | CVYCKQQLLRR | Protein E6 |
| | | | | EVYDFAFRDLCI | |
| | | | | VYRDGNPYA | |
| 110 | ILEGIGILSV | anoctamin-4 isoform 1 | 624 | ELQTTIHDIILEC | Protein E6 |
| | | | | VY | |
| 111 | MNAAVTFANCALGRV | aquaporin-7 isoform 4 | 625 | RDLCIVYRDGN | Protein E6 |
| | | [Homo sapiens] | | PYAVCDKCLKF | |
| | | | | YSKISEYRHY | |
| 112 | PWRKFPVYVLGQFLG | aquaporin-7 isoform 4 | 626 | VYDFAFRDLCIV | Protein E6 |
| | | [Homo sapiens] | | YRD | |
| 113 | INCLSSPNEETVLSA | armadillo repeat- | 627 | FAFKDLCIVY | Protein E6 |
| | | containing protein 7 | | | |
| | | isoform 1 [Homo sapiens] | | | |
| 114 | STAYPAPMRRRCCLP | armadillo repeat- | 628 | FAFSDLYVVY | Protein E6 |
| | | containing protein 7 | | | |
| | | isoform 2 [Homo sapiens] | | | |
| 115 | ILDEKPVII | ATP-binding cassette sub- | 629 | FVFADLRIVY | Protein E6 |
| | | family A member 6 | | | |
| 116 | VLNGTVHPV | ATP-binding cassette sub- | 630 | VAFTEIKIVY | Protein E6 |
| | | family B member 5 | | | |
| 117 | ALIGGPPV | ATP-binding cassette | 631 | DFAFRDLCIVYR | Protein E6 |
| | | transporter A1 | | DGNPYAVCDK | |
| 118 | HIENFSDIDMGE | ATP-dependent RNA | 632 | DKCLKFYSKISE | Protein E6 |
| | | helicase DDX3Y | | YRHYCYSLYG | |
| 119 | VALKPQERVEKRQTP | autism susceptibility gene | 633 | DPQERPRKLPQ | Protein E6 |
| | | 2 protein isoform X9 | | LCTELQTTIHD | |
| | | [Homo sapiens] | | | |
| 120 | TLGEFLKL | Baculoviral IAP repeat- | 634 | HDIILECVYCKQ | Protein E6 |
| | | containing protein 5 | | QLLRREVYDF | |
| 121 | QLCPICRAPV | Baculoviral IAP repeat- | 635 | KQQLLRREVYD | Protein E6 |
| | | containing protein 7 | | FAFRDLCIVYR | |
| 122 | AFLGERVTL | BARF1 protein | 636 | RFHNIRGRWTG | Protein E6 |
| | | | | RCMSCCRSSR | |
| | | | | T | |
| 123 | FLGERVTLT | BARF1 protein | 637 | YRDGNPYAVCD | Protein E6 |
| | | | | KCLKFYSKISE | |
| 124 | KLGPGEEQV | BARF1 protein | 638 | CLKFYSKISEYR | Protein E6 |
| | | | | HYCYSLYGTTL | |
| | | | | EQQYNKPLCD | |
| 125 | RFIAQLLLL | BARF1 protein | 639 | DLFVVYRDSIPH | Protein E6 |
| | | | | AACHKCIDFY | |
| 126 | TLTSYWRRV | BARF1 protein | 640 | FYSRIRELRHYS | Protein E6 |
| | | | | DSVYGDTLEK | |
| 127 | FLLAMTSLR | BcRF1 protein | 641 | GTTLEQQYNKP | Protein E6 |
| | | | | LCDLLIRCINC | |
| 128 | RPKSNIVLL | B-lymphocyte antigen | 642 | KQRHLDKKQRF | Protein E6 |
| | | CD20 | | HNIRGRWTGRC | |
| 129 | RPKSNIVL | B-lymphocyte antigen | 643 | PLCDLLIRCINC | Protein E6 |
| | | CD20 | | QKPLCPEEKQ | |
| 130 | GLCTLVAML | BMLF1 protein | 644 | QERPRKLPQL | Protein E6 |
| 131 | LLGIGILVL | Bone marrow stromal | 645 | QRFHNIRGRW | Protein E6 |
| | | antigen 2 | | | |
| 132 | LLLGIGILVL | Bone marrow stromal | 646 | RWTGRCMSCC | Protein E6 |
| | | antigen 2 | | | |
| 133 | VQLRGRAQGGGALR | brain-specific serine | 647 | SSRTRRETQL | Protein E6 |
| | A | protease 4 precursor | | | |
| | | [Homo sapiens] | | | |
| 134 | HVYDGKFLAR | butyrylcholinesterase | 648 | AFRDLCIVYRD | Protein E6 |
| | | p. Val204Asp splice variant | | GNPY | |
| 135 | GLLSLEEEL | bZIP factor | 649 | HLDKKQRFHNI | Protein E6 |
| | | | | RGRW | |
| 136 | ARRRRRAEKKAADVA | bZIP factor | 650 | FAFRDLCIVYR | Protein E6 |
| | RRKQE | | | | |
| 137 | RRRAEKKAADVA | bZIP factor | 651 | MLDLQPETT | Protein E7 |
| 138 | RAKFKQLL | BZLF1 | 652 | YILDLQPETT | Protein E7 |
| 139 | SPTSSRTSSL | cadherin EGF LAG seven- | 653 | YVLDLQPEAT | Protein E7 |
| | | pass G-type receptor 1 | | | |
| | | precursor | | | |
| 140 | ARAAAAAAFEIDPRS | cadherin EGF LAG seven- | 654 | LEDLLMGTLGIV | Protein E7 |
| | | pass G-type receptor 3 | | CPICSQKP | |
| | | precursor [Homo sapiens] | | | |
| 141 | LLAALVQDYL | calcitonin | 655 | FQQLFLNTL | Protein E7 |
| 142 | CMLGTYTQDF | calcitonin isoform CT | 656 | QLFLNTLSFV | Protein E7 |
| | | preproprotein [Homo | | | |
| | | sapiens] | | | |
| 143 | FLALSILVL | calcitonin isoform CT | 657 | ASDLRTIQQLLM | Protein E7 |
| | | preproprotein [Homo | | GTV | |
| | | sapiens] | | | |
| 144 | GNLSTCMLGTYTQDF | calcitonin isoform CT | 658 | LRTIQQLLMGTV | Protein E7 |
| | | preproprotein [Homo | | NIV | |
| | | sapiens] | | | |
| 145 | MGFQKFSPFLALSIL | calcitonin isoform CT | 659 | MHGDTPTLHEY | Protein E7 |
| | | preproprotein [Homo | | MLDL | |
| | | sapiens] | | | |
| 146 | VLLQAGSLHA | calcitonin isoform CT | 660 | HVDIRTLEDLLM | Protein E7 |
| | | preproprotein [Homo | | GTL | |
| | | sapiens] | | | |
| 147 | SLLMWITQC | Cancer/testis antigen 1 | 661 | DLYCYEQLNDS | Protein E7 |
| | | | | SEEEDEIDGPA | |
| 148 | WITQCFLPVFLAQPP | Cancer/testis antigen 1 | 662 | HYNIVTFCCKC | Protein E7 |
| | SGQRR | | | DSTLRLCVQST | |
| 149 | QLSLLMWIT | Cancer/testis antigen 1 | 663 | IPVDLLCHEQLS | Protein E7 |
| | | | | DSEEENDEID | |
| 150 | APRGPHGGAASGL | Cancer/testis antigen 1 | 664 | IRTLEDLLMGT | Protein E7 |
| 151 | EFTVSGNIL | Cancer/testis antigen 1 | 665 | MHGPKATLQDI | Protein E7 |
| | | | | VLHLEPQNEIP | |
| 152 | LPVPGVLLKEFTVSG | Cancer/testis antigen 1 | 666 | TPTLHEYMLDL | Protein E7 |
| | NILTI | | | QPET | |
| 153 | RGPESRLLEFYLAMP | Cancer/testis antigen 1 | 667 | EMSALLARRRR | Protein enabled |
| | FATPM | | | IAEK | homolog |
| 154 | RLLEFYLAMPFATPM | Cancer/testis antigen 1 | 668 | RVQEAVESMVK | protein |
| | EAELA | | | | FAM136A |
| 155 | VLLKEFTVSGNILTIRL | Cancer/testis antigen 1 | 669 | RYYVGHKGKF | Protein mago |
| | TAA | | | | nashi homolog 2 |
| 156 | DIAGIGLKTV | CAND2 protein | 670 | KIFYVYMKRKY | Protein SSX2 |
| | | | | EAMT | |
| 157 | GAGIGVLTA | carbohydrate-binding | 671 | KASEKIFYV | Protein SSX2 |
| | | protein [Clostridium sp. | | | |
| | | E02] | | | |
| 158 | HLSTAFARV | Carbonic anhydrase 9 | 672 | ATFSSSHRYHK | protein transport |
| | | precursor | | | protein Sec31A |
| | | | | | isoform 7 |
| 159 | LPSQAFEYILYNKG | cathepsin H | 673 | LLHGFSFHL | Protein-serine 0- |
| | | | | | palmitoleoyltran |
| | | | | | sferase |
| | | | | | porcupine |
| 160 | KPGVIFLTKRSRQV | C-C motif chemokine 3 | 674 | ETDPVNHMV | protocadherin |
| | | precursor | | | Fat 2 precursor |
| | | | | | [Homo sapiens] |
| 161 | KVSAVTLAY | CD19 differentiation | 675 | GINQTTGALYLR | protocadherin- |
| | | antigen | | VDS | 16 precursor |
| | | | | | [Homo sapiens] |
| 162 | KLMSPKLYVW | CD19 differentiation | 676 | GLGIGIASM | Proton myo- |
| | | antigen | | | inositol |
| | | | | | cotransporter |
| 163 | VEGSGELFRW | CD19 differentiation | 677 | ALLKDTVYT | proto-oncogene |
| | | antigen | | | PIM1 |
| 164 | STATHSPATTSHGNA | CD68 [Homo sapiens] | 678 | NLVHGPPAPPQ | PSD protein, |
| | | | | VGAD | partial [Homo |
| | | | | | sapiens] |
| 165 | VTVHPTSNSTATSQG | CD68 [Homo sapiens] | 679 | ALYSGVHKK | putative adapter |
| | | | | | and scaffold |
| | | | | | protein |
| 166 | AVGIGIAVV | CD9 antigen | 680 | QLLEGLGFTLTV | PUTATIVE |
| | | | | VPE | PSEUDOGENE: |
| | | | | | RecName: |
| | | | | | Full=Putative |
| | | | | | serpin A13; |
| | | | | | Flags: Precursor |
| 167 | YGYDNVKEY | CDCA7L protein | 681 | EFPVRQAAAIYL | RANBP8 [Homo |
| | | | | K | sapiens] |
| 168 | LQREYASVKEENERL | CDK5 regulatory subunit | 682 | GGGPDGPLYKV | rap1 GTPase- |
| | | associated protein 2 | | SVTA | activating |
| | | [Homo sapiens] | | | protein 1 isoform |
| | | | | | X13 [Homo |
| | | | | | sapiens] |
| 169 | MEVSGCPTPAGQS | CEA cell adhesion | 683 | AAANIIRTL | RASGRF1 |
| | | molecule 18 | | | protein |
| 170 | RTIAPIIGR | cell division cycle 5-like | 684 | LGVIGLVAL | Receptor |
| | | protein | | | expression- |
| | | | | | enhancing |
| | | | | | protein 5 |
| 171 | STPPPGATRV | Cellular tumor antigen p53 | 685 | IISAVVGIL | Receptor |
| | | | | | tyrosine-protein |
| | | | | | kinase erbB-2 |
| | | | | | precursor |
| 172 | HYNYMCNSSCMGGM | Cellular tumor antigen p53 | 686 | KIFGSLAFLPES | Receptor |
| | NRRPILTIITL | | | FDGDPA | tyrosine-protein |
| | | | | | kinase erbB-2 |
| | | | | | precursor |
| 173 | HGLSHSLRQISSQLS | centrosomal protein of 164 | 687 | HPAATHTKAV | Regulatory |
| | | kDa isoform X20 [Homo | | | protein E2 |
| | | sapiens] | | | |
| 174 | RFEEKHAYF | CGI-201 protein | 688 | QIKVRVDMV | regulatory |
| | | | | | protein IE1 |
| 175 | MQLMPFGCLL | Chain A, Epidermal | 689 | TLLQQYCLYL | Replication |
| | | Growth Factor Receptor | | | protein E1 |
| 176 | LIMQLMPFGCL | Chain A, Epidermal | 690 | KSEGEEAQEVE | retinitis |
| | | Growth Factor Receptor | | GETQ | pigmentosa 1- |
| | | | | | like protein 1 |
| | | | | | [Homo sapiens] |
| 177 | YPFKPPKV | Chain B, E2~ubiquitin- | 691 | SSLSLFFRK | retinoblastoma- |
| | | Hect | | | like protein 2 |
| 178 | ETGPEAERLEQLESG | CHL1 protein [Homo | 692 | VRRCLPLCALTL | Rhesus |
| | | sapiens] | | EAA | polypeptide |
| | | | | | RhVIII [Homo |
| | | | | | sapiens] |
| 179 | AVMRWGMPP | Chromodomain-helicase- | 693 | RTNWPNTGK | RINT1 protein, |
| | | DNA-binding protein 3 | | | partial |
| 180 | ILFSLQPGLLRDW | Chromosome 7 open | 694 | GRAPQVLVL | RNA helicase |
| | | reading frame 67 [Homo | | | Il/Gu protein |
| | | sapiens] | | | |
| 181 | MFTLTGCRLVEKT | cilia- and flagella- | 695 | TYSPTSPVYTP | RNA |
| | | associated protein 65 | | TSPK | polymerase II |
| | | isoform X7 [Homo | | | largest subunit |
| | | sapiens] | | | [Homo sapiens] |
| 182 | RLWQELSD | circadian clock protein | 696 | GGHDSSSWSH | RNA-binding |
| | | PASD1 | | RYGGG | motif protein, X- |
| | | | | | linked-like-3 |
| | | | | | [Homo sapiens] |
| 183 | ATAGIIGVNR | CLTC protein | 697 | DPSAIGLADPPI | selenoprotein V |
| | | | | PSP | isoform 2 [Homo |
| | | | | | sapiens] |
| 184 | THRPGGKHGRLAGG | coiled-coil domain- | 698 | AVYTPPSVSTH | serine protease |
| | S | containing protein 74B | | QMPR | 3 |
| | | isoform X8 [Homo sapiens] | | | |
| 185 | SSLPGPPGPPGPPGP | Collagen alpha-1 (XVIII) | 699 | GRLILWEAPPL | serine/threonine |
| | | chain | | GAGG | -protein kinase |
| | | | | | Nek8 [Homo |
| | | | | | sapiens] |
| 186 | EVPMCSDPEPRQEV | constitutive coactivator of | 700 | SIYNNLVSFASP | Serine/threonine |
| | P | peroxisome proliferator- | | LVT | -protein kinase |
| | | activated receptor gamma | | | SMG1 |
| | | isoform X2 [Homo | | | |
| | | sapiens] | | | |
| 187 | VTTKKTPPSQPPGNV | Contactin 3 | 701 | LLLELAGVTHV | Signal- |
| | | (plasmacytoma | | | regulatory |
| | | associated) [Homo | | | protein delta |
| | | sapiens] | | | |
| 188 | YLNKEIEEA | CTD (carboxy-terminal | 702 | NLKLKLHSF | Similar to |
| | | domain, RNA polymerase | | | retinoblastoma- |
| | | Il, polypeptide A) | | | binding protein |
| | | phosphatase, subunit 1 | | | 4, partial |
| 189 | ARGGLVDEKALAQAL | C-terminal- | 703 | TRLFPNEFANF | SLIT and NTRK- |
| | KEGRIRGAAL | binding protein | | YNAV | like protein 1 |
| | | 1 | | | precursor [Homo |
| | | | | | sapiens] |
| 190 | ALTPVVVTL | cyclin-dependent kinase 4 | 704 | CISCKLSRQHC | small t antigen |
| | | | | SLKTLKQKN | [Merkel cell |
| | | | | | polyomavirus] |
| 191 | DFGFARTLAAPGDI | cyclin-dependent kinase- | 705 | KLGIGFAKA | sodium channel |
| | | like 3 isoform X10 [Homo | | | alpha subunit |
| | | sapiens] | | | |
| 192 | INFKIERGQLLAV | cystic fibrosis | 706 | FLQEVNVYGV | Solute carrier |
| | | transmembrane | | | family 27 |
| | | conductance regulator | | | member 3 |
| | | [Homo sapiens] | | | |
| 193 | GLGIGALVL | Cytochrome c oxidase | 707 | LIGIGLNLV | Spatacsin |
| | | assembly factor 3 | | | |
| | | homolog, mitochondrial | | | |
| 194 | QWIKYFDKRRDYLKF | Cytochrome c oxidase | 708 | IEELRHLWDLLL | spectrin alpha |
| | | assembly factor 6 | | ELTL | chain, |
| | | homolog | | | erythrocytic 1 |
| | | | | | isoform X4 |
| | | | | | [Homo sapiens] |
| 195 | FSISQLQKNHDMNDE | cytochrome P450 7B1 | 709 | KELRALRKMVS | spermatid- |
| | | isoform 1 [Homo sapiens] | | NMSG | associated |
| | | | | | protein isoform |
| | | | | | X1 [Homo |
| | | | | | sapiens] |
| 196 | AVKRLPLVYCDYHGH | cytosolic | 710 | YTCPLCRAPV | SSA protein SS- |
| | | carboxypeptidase 1 | | | 56 |
| | | isoform X8 [Homo | | | |
| | | sapiens] | | | |
| 197 | MLLDKNIPI | DAPK2 protein | 711 | IMLEGETKL | ssDNA-binding |
| | | | | | phosphoprotein |
| 198 | IAGPGTITL | diaminopimelate | 712 | QQEIDQKRLEF | structural |
| | | decarboxylase | | EKQK | maintenance of |
| | | | | | chromosomes |
| | | | | | protein 1B |
| | | | | | isoform X2 |
| | | | | | [Homo sapiens] |
| 199 | SSSGLHPPK | Dmx-like 1, isoform | 713 | AGRFGQGAHH | suprabasin |
| | | CRA_a | | AAGQA | isoform X3 |
| | | | | | [Homo sapiens] |
| 200 | VTEHDTLLY | DNA processivity factor | 714 | RLFVGSIPK | SYNCRIP |
| | | | | | protein, partial |
| 201 | SSAEPTEHGERTPLA | DPCR1 [Homo sapiens] | 715 | GYDQIMPKK | SYT-SSX |
| | | | | | protein |
| 202 | KPRQSSPQL | dynamin binding protein, | 716 | APLQRSQSL | TBC1 domain |
| | | isoform CRA_b | | | family, member |
| | | | | | 22A, isoform |
| | | | | | CRA b |
| 203 | GLYYVHEGIRTYFVQ | Regulatory protein E2 | 717 | EPGDTALYLCA | T cell receptor |
| | | | | SSQS | beta chain |
| | | | | | variable region, |
| | | | | | partial [Homo |
| | | | | | sapiens] |
| 204 | YLTAPTGCI | Regulatory protein E2 | 718 | ALLTSRLRFIP | Telomerase |
| | | | | | reverse |
| | | | | | transcriptase |
| 205 | DSAPILTAF | Regulatory protein E2 | 719 | EARPALLTSRL | Telomerase |
| | | | | RFIPK | reverse |
| | | | | | transcriptase |
| 206 | ILTAFNSSHK | Regulatory protein E2 | 720 | LLTSRLRF | Telomerase |
| | | | | | reverse |
| | | | | | transcriptase |
| 207 | KSAIVTLTY | Regulatory protein E2 | 721 | LLTSRLRFI | Telomerase |
| | | | | | reverse |
| | | | | | transcriptase |
| 208 | LAVSKNKAL | Regulatory protein E2 | 722 | RPALLTSRLRFI | Telomerase |
| | | | | | reverse |
| | | | | | transcriptase |
| 209 | LQDVSLEVY | Regulatory protein E2 | 723 | GTADVHFER | THO complex |
| | | | | | subunit 4 |
| 210 | LTAPTGCIKK | Regulatory protein E2 | 724 | ASFDKAKLK | thymosin beta- |
| | | | | | 10, partial |
| 211 | NPCHTTKLL | Regulatory protein E2 | 725 | PTKCEVERFTA | thyroglobulin |
| | | | | TSFG | isoform X10 |
| | | | | | [Homo sapiens] |
| 212 | NTTPIVHLK | Regulatory protein E2 | 726 | ESDPIVAQY | Titin |
| 213 | QVILCPTSV | Regulatory protein E2 | | | |
| 214 | RLECAIYYK | E2 | 728 | GDCVQGDWCP | transcriptional |
| | | | | ISGGL | activator Tax |
| 215 | SPEIIRQHL | E2 | 729 | ISGGLCSARLH | transcriptional |
| | | | | RHAL | activator Tax |
| 216 | TLKCLRYRFK | E2 | 730 | LLFGYPVYV | transcriptional |
| | | | | | activator Tax |
| 217 | TLYTAVSST | E2 | 731 | SFHSLHLLF | transcriptional |
| | | | | | activator Tax |
| 218 | VVEGQVDYY | E2 | 732 | VVCMYLYQLSP | transcriptional |
| | | | | PITW | activator Tax |
| 219 | YRFKKHCTL | E2 | 733 | AGQNPASHPPP | transcriptional |
| | | | | DDAE | adapter 1 [Homo |
| | | | | | sapiens] |
| 220 | YYVHEGIRTY | E2 | 734 | RVEYHFLSPYV | transferrin |
| | | | | SPK | receptor protein |
| | | | | | 1 |
| 221 | ALQAIELQL | E2 protein | 735 | NSVIIVDKNGRL | transferrin |
| | | | | V | receptor protein |
| | | | | | 1 |
| 222 | TLQDVSLEV | E2 protein | 736 | ILLGIGIYAL | transmembrane |
| | | | | | and coiled-coil |
| | | | | | domain- |
| | | | | | containing |
| | | | | | protein 2 [Homo |
| | | | | | sapiens] |
| 223 | YICEEASVTV | E2 protein | 737 | LLGIGIYAL | transmembrane |
| | | | | | and coiled-coil |
| | | | | | domain- |
| | | | | | containing |
| | | | | | protein 2 [Homo |
| | | | | | sapiens] |
| 224 | CQDKILTHYENDSTD | E2 protein | 738 | IHSSWDCGLFT | transmembrane |
| | | | | NYSA | channel-like |
| | | | | | protein 8 isoform |
| | | | | | X10 [Homo |
| | | | | | sapiens] |
| 225 | EEASVTVVEGQVDYY | E2 protein | 739 | QLLDQVEQI | transmembrane |
| | | | | | emp24 domain- |
| | | | | | containing |
| | | | | | protein 4 isoform |
| | | | | | 4 |
| 226 | QVILCPTSVFSSNEV | E2 protein | 740 | IMASKGMRHFC | transmembrane |
| | | | | LISE | protein 168 |
| | | | | | isoform X2 |
| | | | | | [Homo sapiens] |
| 227 | TEETQTTIQRPRSEP | E2 protein | 741 | MRHFCLISE | transmembrane |
| | | | | | protein 168 |
| | | | | | isoform X2 |
| | | | | | [Homo sapiens] |
| 228 | VLFYLGQYI | E3 ubiquitin-protein ligase | 742 | IAGLGLLTV | transmembrane |
| | | Mdm2 | | | protein 74B |
| | | | | | isoform 1 |
| 229 | DEKQQHIVY | E3 ubiquitin-protein ligase | 743 | GELIGTLNAAKV | triosephosphate |
| | | Mdm2 | | PAD | isomerase 1 |
| 230 | DEVYQVTVY | E3 ubiquitin-protein ligase | 744 | QVATEGLAK | Tripartite |
| | | Mdm2 | | | terminase |
| | | | | | subunit UL28 |
| | | | | | homolog |
| 231 | PSRHRYGARQPRAR | E3 ubiquitin-protein ligase | 745 | RLARLALVL | Trophoblast |
| | L | RNF126 isoform 1 [Homo | | | glycoprotein |
| | | sapiens] | | | |
| 232 | LGLWRGEEVTLSNPK | E3 ubiquitin-protein ligase | 746 | PNGTSVPRNSS | truncated large |
| | | TRIP12 isoform X5 [Homo | | RTYGTWEDL | T antigen |
| | | sapiens] | | | [Merkel cell |
| | | | | | polyomavirus] |
| 233 | LWTEGMLQMAFHILA | E3 ubiquitin-protein ligase | 747 | MRETSGFTL | trypsin-3 isoform |
| | | UBR1 [Homo sapiens] | | | 3 preproprotein |
| | | | | | [Homo sapiens] |
| 234 | LLSVSTYTSLILLVL | E5 | 748 | AVFDGAQVTSK | tumor protein |
| | | | | | p53 inducible |
| | | | | | protein 11, |
| | | | | | isoform CRA_c |
| 235 | YIIFVYIPL | E5 protein | 749 | YMDGTMSQV | Tyrosinase |
| 236 | FAFRDLCIV | E6 | 750 | MLLAVLYCL | Tyrosinase |
| | | | | | precursor |
| 237 | IILECVYCK | E6 | 751 | YMNGTMSQV | Tyrosinase |
| | | | | | precursor |
| 238 | LLIRCINCQK | E6 | 752 | FQDYIKSYL | Tyrosinase |
| | | | | | precursor |
| 239 | PYAVCDKCL | E6 | 753 | SVYDFFVWL | tyrosinase- |
| | | | | | related protein-2 |
| 240 | TTLEQQYNK | E6 | 754 | PKPVLHMVSSE | Tyrosine-protein |
| | | | | QHSA | phosphatase |
| | | | | | non-receptor |
| | | | | | type 12 |
| 241 | VCDKCLKFY | E6 | 755 | YLAPENGYL | U1 small nuclear |
| | | | | | ribonucleoprotein |
| | | | | | 70 kDa |
| 242 | CPEEKQRHL | E6 | 756 | KMGIGWKPL | Uncharacterized |
| | | | | | protein C2orf71 |
| 243 | QYNKPLCDL | E6 | 757 | HYYVSMDAL | unknown protein |
| | | | | | eluted from |
| | | | | | human MHC |
| | | | | | allele |
| 244 | FAFSDLCIVY | E6 | 758 | KYKDRTNILF | unknown protein |
| | | | | | eluted from |
| | | | | | human MHC |
| | | | | | allele |
| 245 | LKFYSKISEYRHYCY | E6 | 759 | RLPSSADVEF | unknown protein |
| | | | | | eluted from |
| | | | | | human MHC |
| | | | | | allele |
| 246 | AVCDKCLKFY | E6 | 760 | RYNADISTF | unknown protein |
| | | | | | eluted from |
| | | | | | human MHC |
| | | | | | allele |
| 247 | CVYCKQQLLR | E6 | 761 | QEEKSLDPMVY | unnamed |
| | | | | MNDTSPLT | protein product |
| 248 | NPYAVCDKCL | E6 | 762 | TCSCQSSGTSS | unnamed |
| | | | | TSYS | protein product |
| | | | | | [Homo sapiens] |
| 249 | RPRKLPQLCT | E6 | 763 | TIYSLFYSVADR | unnamed |
| | | | | DAPA | protein product |
| | | | | | [Homo sapiens] |
| 250 | YAVCDKCLKF | E6 | 764 | GAIVIERPNVKW | vacuolar protein |
| | | | | S | sorting- |
| | | | | | associated |
| | | | | | protein 4B |
| | | | | | [Homo sapiens] |
| 251 | YGTTLEQQY | E6 | 765 | DIMRVNVDKVL | vesicle- |
| | | | | ERDQKL | associated |
| | | | | | membrane |
| | | | | | protein 3 |
| 252 | CYSLYGTTL | E6 protein | 766 | VDKVLERDQKL | vesicle- |
| | | | | SELDDR | associated |
| | | | | | membrane |
| | | | | | protein 3 |
| 253 | FAFRDLCIVY | E6 protein | 767 | SSVPGVRLL | vimentin |
| 254 | EYRHYCYSL | E6 protein | | NYIDKVR | vimentin |
| 255 | PRKLPQLCTELQTTI | E6 protein | 769 | CQWGRLWQL | WD repeat- |
| | | | | | containing |
| | | | | | protein BING4 |
| 256 | QYNKPLCDLL | E6 protein | 770 | MCQWGRLWQL | WD repeat- |
| | | | | | containing |
| | | | | | protein BING4 |
| 257 | VYDFAFQDL | E6 protein | 771 | CMTWNQMNL | Wilms tumor |
| | | | | | protein |
| 258 | FACYDLCIVY | E6 protein | 772 | RMFPNAPYL | Wilms tumor |
| | | | | | protein |
| 259 | ILIRCIICQ | E6 protein | 773 | SGSGSGPLPSL | zinc finger |
| | | | | FLNS | homeodomain |
| | | | | | protein [Homo |
| | | | | | sapiens] |
| 260 | KCLNEILIR | E6 protein | 774 | LTDHRAHRCPG | zinc finger |
| | | | | GNAK | protein |
| 261 | KVCLRLLSK | E6 protein | 775 | QLTAHKMIHTG | Zinc finger |
| | | | | EKPY | protein 100 |
| | | | | | [Homo sapiens] |
| 262 | GIVCPICSQK | E7 | 776 | ECSECGKVFLE | zinc finger |
| | | | | SAAL | protein 264 |
| | | | | | [Homo sapiens] |
| 263 | IVCPICSQK | E7 | 777 | EECGKAFSVFS | zinc finger |
| | | | | TLTK | protein 273 |
| | | | | | isoform X1 |
| | | | | | [Homo sapiens] |
| 264 | QAEPDRAHY | E7 | 778 | VTGGRGGRQG | Zinc finger |
| | | | | PSPAF | protein 385C |
| | | | | | [Homo sapiens] |
| 265 | QSTHVDIRTLEDLLM | E7 | 779 | CNFSTIDVVSLK | zinc finger |
| | GTLGI | | | TDT | protein 407 |
| | | | | | isoform X2 |
| | | | | | [Homo sapiens] |
| 266 | RAHYNIVTF | E7 | 780 | LTDHRAHRCPG | zinc finger |
| | | | | DGDD | protein 423 |
| | | | | | isoform 2 [Homo |
| | | | | | sapiens] |
| 267 | TLGIVCPI | E7 | 781 | SNLTKHKKIHIE | zinc finger |
| | | | | KKP | protein 43 |
| | | | | | isoform 1 [Homo |
| | | | | | sapiens] |
| 268 | AGQAEPDRAHYNIVT | E7 | 782 | IHKMIHTGEKPY | zinc finger |
| | FCCKCDSTLRLCVQS | | | KCE | protein 493 |
| | THVDI | | | | isoform 3 [Homo |
| | | | | | sapiens] |
| 269 | MHGDTPTLHEYMLDL | E7 | 783 | LQNHIQTIHREL | zinc finger |
| | QPETTDLYCYEQLND | | | VPD | protein 521 |
| | SS | | | | isoform 1 [Homo |
| | | | | | sapiens] |
| 270 | TDLYCYEQLNDSSEE | E7 | 784 | NVAKPSSGPHT | zinc finger |
| | EDEIDGPAGQAEPDR | | | LLHI | protein 626 |
| | AHYNIV | | | | isoform 1, partial |
| | | | | | [Homo sapiens] |
| 271 | LFMDSLNFVCPWC | E7 | 785 | SNLTTHKKIHTG | zinc finger |
| | | | | ERP | protein 626 |
| | | | | | isoform 1, partial |
| | | | | | [Homo sapiens] |
| 272 | EIDGPAGQAEPDRAH | E7 | 786 | EKPYSCPDCSL | zinc finger |
| | YNI | | | RFAY | protein 785 |
| | | | | | [Homo sapiens] |
| 273 | GVNHQHLPARRAEP | E7 | 787 | KCEECDTVFSR | zinc finger |
| | Q | | | KSHH | protein 860 |
| | | | | | [Homo sapiens] |
| 274 | HGDTPTLHEY | E7 | 788 | GCGKVFARSEN | zinc finger |
| | | | | LKIH | protein ZIC 4 |
| | | | | | isoform 3 [Homo |
| | | | | | sapiens] |
| 275 | HYNIVTFCCK | E7 | 789 | RYQQWMERF | zinc |
| | | | | | phosphodiestera |
| | | | | | se ELAC protein |
| | | | | | 2 isoform 3 |
| 276 | LMGTLGIVCPI | E7 | 790 | SAGPPSLRK | ZMYM4 protein |
| 277 | MLDLQPETTDLYCYE | E7 | 791 | PMEKPTISTEKP | zonadhesin |
| | | | | TIP | isoform X1 |
| | | | | | [Homo sapiens] |
| 278 | TLRLCVQSTHVDIRT | E7 | 792 | APIWPYEILY | |
| 279 | LQPETTDLY | E7 | 793 | CYTWNQMNL | |
| 280 | TPTLHEYML | E7 | 794 | DAEKSDICTDE | |
| | | | | Y | |
| 281 | DRAHYNIVTFCCKCD | E7 protein | 795 | ELAGIGILTV | |
| 282 | DSTLRLCVQSTHVD | E7 protein | 796 | IMDQVPFSV | |
| 283 | GTLGIVCPI | E7 protein | 797 | KLCPVQLWV | |
| 284 | HVDIRTLED | E7 protein | 798 | LMLGEFLKL | |
| 285 | LLMGTLGIV | E7 protein | 799 | SLLMWITQA | |
| 286 | MHGDTPTLHEYM | E7 protein | 800 | SLLMWITQV | |
| 287 | TLGIVCPIC | E7 protein | 801 | SLPPPGTRV | |
| 288 | TLHEYMLDL | E7 protein | 802 | TLGIVCPI + | |
| | | | | AIB(C6) | |
| 289 | YMLDLQPET | E7 protein | 803 | VLPDVFIRC | |
| 290 | YMLDLQPETT | E7 protein | 804 | YLEPGPVTL | |
| 291 | DLLMGTLGIVCPICSQ | E7 protein | 805 | YLEPGPVTV | |
| | KP | | | | |
| 292 | HYNIVTFCCKCDSTLR | E7 protein | 806 | YLEPGPVTVP | |
| | LCVQ | | | | |
| 293 | LRLCVQSTHVDIRTLE | E7 protein | 807 | YLGSYGFRL | |
| | DLLM | | | | |
| 294 | CCKCDSTL | E7 protein | 808 | GELIGILNAAKV | |
| | | | | PAD | |
| 295 | STHVDIRTLEDLLMG | E7 protein | 809 | ALGIGILTV | |
| 296 | CDSTLRLCVQSTHVDI | E7 protein | — | 2,4-dinitrophenyl | |
| | RTLE | | | group | |
| 297 | ATEVRTLQQ | E7 protein | 810 | KAVAAWTLKAA | |
| 298 | CTIVCPSCA | E7 protein | 811 | FATGIGIITV | |
| 299 | LCINSTATE | E7 protein | 812 | FLTGIGIITV | |
| 300 | CYEQLGDSS | early protein | — | 6-formylpterin | |
| 301 | ITIRCIICQ | early protein | 813 | ALAGIGILTV | |
| 302 | KTLEERVKK | early protein | 814 | ELAAIGILTV | |
| 303 | MRGDKATIK | early protein | 815 | ELAGIGILAV | |
| 304 | PYGVCIMCL | early protein | — | 5-(2- | |
| | | | | oxopropylidene | |
| | | | | amino)-6-D- | |
| | | | | ribityl | |
| | | | | aminouracil | |
| 305 | QLGDSSDEE | early protein | 816 | GVAGIGILTG + | |
| | | | | MCM(G1, V2, | |
| | | | | G10) | |
| 306 | RLQCVQCKK | early protein | 817 | SAAGIGILTV + | |
| | | | | MCM(S1, A2) | |
| 307 | VYKFLFTDL | early protein | 818 | SVYDFFVWL + | |
| | | | | MCM(S1, V2) | |
| 308 | GGSKTSLYNLRRGTA | EBNA-1 | — | N(2)-acetyl-6- | |
| | | | | formylpterin | |
| 309 | HPVGEADYFEY | EBNA-1 | 819 | ALEPGPVTA | |
| 310 | RPSCIGCKGTHGGTG | EBNA-1 | 820 | YLEAGPVTA | |
| 311 | NPKFENIAEGLRALLA | EBNA-1 protein | 821 | YLEPGAVTA | |
| | RSHVERTTDE | | | | |
| 312 | PGTGPGNGLGEKGD | EBNA-1 protein | 822 | YLEPGPATA | |
| | T | | | | |
| 313 | PPMVEGAAAEGDDG | EBNA-1 protein | 823 | YLEPGPVAA | |
| | D | | | | |
| 314 | VLKDAIKDL | EBNA-1 protein | 824 | AMFWSVPTV | |
| 315 | YFMVFLQTHIFAEVL | EBNA-1 protein | 825 | CLNEYHLFL | |
| 316 | RLRAEAQVK | EBNA3A nuclear protein | 826 | EYYSKNLNSF | |
| 317 | RPPIFIRRL | EBNA3A nuclear protein | 827 | FLYNLLTRV | |
| 318 | RYSIFFDY | EBNA3A nuclear protein | 828 | GLGPGFSSY | |
| 319 | RYSIFFDYM | EBNA3A nuclear protein | 829 | HLYASLSRV | |
| 320 | VQPPQLTQV | EBNA3A nuclear protein | 830 | IILVAVPHV | |
| 321 | VSFIEFVGW | EBNA-3B nuclear protein | 831 | KLMNIQQKL | |
| 322 | RRIYDLIEL | EBNA3C latent protein | 832 | KMIGNHLWV | |
| 323 | VAGCYLKYKKKNSLS | EF-hand calcium-binding | 833 | MLGEQLFPL | |
| | | domain-containing protein | | | |
| | | 13 | | | |
| 324 | DPKDAEKAI | ELAV-like protein 4 | 834 | QLSCISTYV | |
| | | (Paraneoplastic | | | |
| | | encephalomyelitis antigen | | | |
| | | HuD) (Hu-antigen D) | | | |
| 325 | GPFGAVNNV | ELAV-like protein 4 | 835 | TYLPSAWNF | |
| | | (Paraneoplastic | | | |
| | | encephalomyelitis antigen | | | |
| | | HuD) (Hu-antigen D) | | | |
| 326 | KPSGATEPI | ELAV-like protein 4 | 836 | TYLPSAWNFF | |
| | | (Paraneoplastic | | | |
| | | encephalomyelitis antigen | | | |
| | | HuD) (Hu-antigen D) | | | |
| 327 | PPSACSPRF | ELAV-like protein 4 | 837 | VYQYTFPDF | |
| | | (Paraneoplastic | | | |
| | | encephalomyelitis antigen | | | |
| | | HuD) (Hu-antigen D) | | | |
| 328 | SPRFSPITI | ELAV-like protein 4 | 838 | VYQYTFPDFL | |
| | | (Paraneoplastic | | | |
| | | encephalomyelitis antigen | | | |
| | | HuD) (Hu-antigen D) | | | |
| 329 | LADGEGGGTDEGIYD | embryonal Fyn-associated | 839 | YQYTFPDF | |
| | | substrate isoform X2 | | | |
| | | [Homo sapiens] | | | |
| 330 | YVVPPPARPCPTSGP | embryonal Fyn-associated | 840 | YQYTFPDFLY | |
| | | substrate isoform X2 | | | |
| | | [Homo sapiens] | | | |
| 331 | VLACGLSRIWGEERG | endothelin receptor type B | 841 | YSKNLNSFF | |
| | | isoform 2 precursor [Homo | | | |
| | | sapiens] | | | |
| 332 | LDHILEPSIPWKSK | envelope glycoprotein | 842 | YYSKNLNSF | |
| 333 | FTQEVSRLNINLHFSK | envelope glycoprotein | 8432 | YYSKNLNSFF | |
| | CGFPF | | | | |
| 334 | GGYYSASYSDPCSLK | envelope glycoprotein | 844 | AVCPWTWLR | |
| | CPYLGC | | | | |
| 335 | HILEPSIPWKSKLLT | envelope glycoprotein | 845 | APARLERRHSA | |
| 336 | LPFNWTHCFDPQIQAI | envelope glycoprotein | 846 | GEEDGAGGHS | |
| | VSSPC | | | L | |
| 337 | SNLDHILEPSIPWK | envelope glycoprotein | 847 | GLLDEDFYA | |
| 338 | SQLPPTAPPLLPHSNL | envelope glycoprotein | 848 | GQFLTPNSH | |
| | DHILEPSIPWKSKL | | | | |
| 339 | GAGIGVAVL | Envelope glycoprotein C | 849 | HQNPVTGLLL | |
| 340 | IAGIGILAI | envelope glycoprotein C | 850 | RHDLPPYRVYL | |
| 341 | GIGIGSGQL | Epsin-3 | 851 | RKTVRARSRTP | |
| | | | | SCRSRSHTPSR | |
| | | | | RRR | |
| 342 | VFLQTHIFAEVLKDA | Epstein-Barr nuclear | 852 | RVSTLRVSL | |
| | IKDLV | antigen 1 | | | |
| 343 | ELFQDLSQL | ETS translocation variant | 853 | SWISDIRAGTAP | |
| | | 5 | | LCRNHIKSSCSL | |
| | | | | I | |
| 344 | IGGIGTVPV | Eukaryotic translation | 854 | SYMIMEIEL | |
| | | elongation factor 1 alpha 1 | | | |
| 345 | TMLARLVSDS | FAM161 centrosomal | 855 | TLWCSPIKV | |
| | | protein A | | | |
| 346 | GTSSVIVSR | F-box/WD repeat- | 856 | VLLGVKLFGV | |
| | | containing protein 11 | | | |
| | | isoform A | | | |
| 347 | RINEFSISSF | fibromodulin | 857 | WLIRETQPITK | |
| 348 | DEVSMKGRPPPTPLF | FK506-binding protein 15 | 858 | YHSIEWAI | |
| | | isoform X3 [Homo | | | |
| | | sapiens] | | | |
| 349 | YLCSGSSYF | Fragile X mental | 859 | EAKPSRILM | |
| | | retardation 1 neighbor | | | |
| | | protein | | | |
| 350 | YLCSGSSYFV | Fragile X mental | 860 | GIAARVKNWL | |
| | | retardation 1 neighbor | | | |
| | | protein | | | |
| 351 | ALGIGAVPI | Full-length cDNA clone | 861 | KEGIAARVKNW | |
| | | CSODI011YN22 of | | | |
| | | Placenta of Homo sapiens | | | |
| | | (human) | | | |
| 352 | SQNPRFYHK | G protein pathway | 862 | WEAKPSRIL | |
| | METH(R5) | suppressor 2 | | | |
| 353 | ALFDIESKV | Glutamate | 863 | AARGPHGGAA | |
| | | carboxypeptidase 2 | | SGL | |
| 354 | MQLIPDDYSNTHSTR | glycoprotein B | 864 | APAGPHGGAAS | |
| | YVTVK | | | GL | |
| 355 | EYILSLEEL | glypican 3 | 865 | APRGAHGGAA | |
| | | | | SGL | |
| 356 | FVGEFFTDV | glypican 3 | 866 | APRGPAGGAAS | |
| | | | | GL | |
| 357 | AVYGQKEIHRK | GTPase activating protein | 867 | APRGPHAGAAS | |
| | | (SH3 domain) binding | | GL | |
| | | protein 1 | | | |
| 358 | KLVVVGAGGVGKSAL | GTPase KRas isoform a | 868 | APRGPHGGAA | |
| | | [Homo sapiens] | | AGL | |
| 359 | VYLDKFIRL | guanine nucleotide- | 869 | APRGPHGGAA | |
| | | binding protein-like 3-like | | SAL | |
| | | protein | | | |
| 360 | FKQDLMIEDNLL | hCG16256, isoform | 870 | APRGPHGGAA | |
| | | CRA_a [Homo sapiens] | | SGA | |
| 361 | ALKIKGIHPYHSLSY | hCG1810774, isoform | 871 | GRIAFFLKY | |
| | | CRA_c [Homo sapiens] | | | |
| 362 | TPEPAIPPKATLWPA | hCG2000808, partial | 872 | KLILWRGLK | |
| | | [Homo sapiens] | | | |
| 363 | LLDVAPLSL | Heat shock 70 kDa protein | 873 | ILEDRGFNQV | |
| | | 1 | | | |
| 364 | LLLLDVAPL | Heat shock 70 kDa protein | 874 | IMEDVGWLNV | |
| | | 1 | | | |
| 365 | TYLPTNASL | HER2 receptor | 875 | LMFDRGMSLL | |
| 366 | VLHDDLLEA | histocompatibility (minor) | 876 | MMWDAGLGM | |
| | | HA-1 | | M | |
| 367 | LEKQLIEL | HMMR protein | 877 | MMWDRGAGM | |
| | | | | M | |
| 368 | SVSPVVHVR | HNRPLL protein | 878 | MMWDRGLGAM | |
| 369 | DKKIEPRGPTIKPCPP | immunoglobulin gamma | 879 | MMWDRGLGM | |
| | CKCP | 2A chain | | | |
| 370 | RGPTIKPCPPCKCP | immunoglobulin gamma | 880 | MMWDRGLGM | |
| | | 2A chain | | M | |
| 371 | LGIGVITI | Immunoglobulin heavy | 881 | MMWDRGMGLL | |
| | | chain | | | |
| 372 | GYGEMGSGYREDLG | immunoglobulin-like and | 882 | NLSNLGILV | |
| | A | fibronectin type Ill domain- | | | |
| | | containing protein 1 | | | |
| | | isoform X3 [Homo | | | |
| | | sapiens] | | | |
| 373 | ETDPLTFNF | inactive phospholipase D5 | 883 | NMGGLGIMPV | |
| | | [Homo sapiens] | | | |
| 374 | LQPYYGFSNQEVIEM | Inactive tyrosine-protein | 884 | SMAGIGIVDV | |
| | VRKRQ | kinase transmembrane | | | |
| | | receptor ROR1 | | | |
| 375 | NKSQKPYKI | Inactive tyrosine-protein | 885 | SMLGIGIVPV | |
| | | kinase transmembrane | | | |
| | | receptor ROR1 | | | |
| 376 | SLSASPVSN | Inactive tyrosine-protein | 886 | CMWGRLWQL | |
| | | kinase transmembrane | | | |
| | | receptor ROR1 | | | |
| 377 | TMIGTSSHL | Inactive tyrosine-protein | 887 | NLSNLGILPV | |
| | | kinase transmembrane | | | |
| | | receptor ROR1 | | | |
| 378 | VATNGKEVV | Inactive tyrosine-protein | 888 | SLANIGILPV | |
| | | kinase transmembrane | | | |
| | | receptor ROR1 | | | |
| 379 | NLSALGIFST | Insulin-like growth factor 2 | 889 | ITSAIGILPV | |
| | | mRNA-binding protein 2 | | | |
| 380 | EFESAQFPNWYISTS | Interleukin-1 beta | 890 | ITSAIGVLFV | |
| 381 | YKAFSSLLASSAVS | interleukin-4 receptor alfa | 891 | ITSAIGVLPI | |
| | PE | chain | | | |
| 382 | LIGIGIAPL | iron-responsive element- | 892 | ITSAIGVLPV | |
| | | binding protein 2 | | | |
| 383 | LVLILYLCV | K8.1 | 893 | ITSGIGVLPV | |
| 384 | LLDRGSFRNDGLKAS | kalirin isoform 2 [Homo | 894 | LTSAIGVLPV | |
| | | sapiens] | | | |
| 385 | ARVILGVRWYVETTS | kallikrein 6 (neurosin, | 895 | MTSAIGILPV | |
| | | zyme), isoform CRA_c, | | | |
| | | partial [Homo sapiens] | | | |
| 386 | CQGDSGGPLVCGDH | kallikrein-related | 896 | MTSAIGVLPV | |
| | L | peptidase 6 transcript | | | |
| | | variant 10 [Homo sapiens] | | | |
| 387 | KPVILGVRWYVETTS | kallikrein-related | 897 | QTSAIGILPV | |
| | | peptidase 6 transcript | | | |
| | | variant 6 [Homo sapiens] | | | |
| 388 | SQLMLTRKAEAALRK | KAT8 regulatory NSL | 898 | QTSAIGVLPV | |
| | | complex subunit 1 isoform | | | |
| | | X6 [Homo sapiens] | | | |
| 389 | VPVAQVTTTSTTDAD | keratin associated protein | 899 | FILLLFLTIFI | |
| | | [Homo sapiens] | | | |
| 390 | CRPQCCQSVCCQPT | keratin associated protein | 900 | FISIFFFLEI | |
| | C | 4.14 [Homo sapiens] | | | |
| 391 | PRCCISSCCRPSCCV | keratin associated protein | 901 | FMDMAILVES | |
| | | 4.14 [Homo sapiens] | | | |
| 392 | SSGGGSSGGGYGGG | Keratin, type I cytoskeletal | 902 | ILLLFLTIFI | |
| | S | 10 | | | |
| 393 | TCCRTTCYRPSCCVS | Keratin-associated protein | 903 | LLFLTIFIYA | |
| | | 4-7 | | | |
| 394 | AFTLLLYCELLQWED | KIAA0299, partial [Homo | 904 | LLLFLTIFI | |
| | | sapiens] | | | |
| 395 | ENKNQELRSLISQYQ | KIAA0299, partial [Homo | 905 | RLMVAVEEA | |
| | | sapiens] | | | |
| 396 | NVSFFHYPEYGY | KIAA1455 protein, partial | 906 | RLMVAVEEAFI | |
| | | [Homo sapiens] | | | |
| 397 | RKFISLHRKALESDF | KIAA1607 protein, partial | 907 | DLAGIGILTV | |
| | | [Homo sapiens] | | | |
| 398 | LQIEEEYQV | L protein | 908 | EIAGIGILTV | |
| 399 | IHSMNSSIL | L1 | 909 | ELAGIGIITV | |
| 400 | NVFPIFLQM | L1 | 910 | ELAGIGILSV | |
| 401 | AVPDDLYIK | L1 | 911 | ELAGIGILTA | |
| 402 | KYTFWEVNL | L1 | 912 | ELAGIGILTI | |
| 403 | IHSMNSTIL | L1 protein | 913 | ELAGIGLLTV | |
| 404 | NLASSNYFPTPSGSM | L1 protein | 914 | ELGGIGILTV | |
| 405 | PSGSMVTSDAQIFNK | L1 protein | 915 | AYRDLQTRK | |
| 406 | GNIPLMKAAFKRSCL | large T antigen [Merkel | 916 | EGTKGATMDLE | |
| | KHHPD | cell polyomavirus] | | IVNLPNVEISKD | |
| | | | | LS | |
| 407 | MDLVLNRKEREAL | large T antigen [Merkel | 917 | EWDPLDIAFET | |
| | | cell polyomavirus] | | WDIIFRNMNKE | |
| | | | | DEG | |
| 408 | NSGRESSTPNGTSVP | large T antigen [Merkel | 918 | GKAFGLCSIFTE | |
| | RNSSR | cell polyomavirus] | | QKKIFSREKCY | |
| | | | | KC | |
| 409 | PVIMMELNTLWSK | large T antigen [Merkel | 919 | HYNYMCNSSC | |
| | | cell polyomavirus] | | MGSMNRRPILTI | |
| | | | | ITL | |
| 410 | TLWSKFQQNIHKL | large T antigen [Merkel | 920 | ILQYLDSERRQI | |
| | | cell polyomavirus] | | RIAKSPLAPFTS | |
| | | | | F | |
| 411 | WEDLFCDESLSSP | large T antigen [Merkel | 921 | KQHVCGGSILD | |
| | | cell polyomavirus] | | PYWVLTAAHCF | |
| | | | | RKH | |
| 412 | WEDLFCDESLSSPEP | large T antigen [Merkel | 922 | LGSRELFCSKL | |
| | PSSSE | cell polyomavirus] | | RRAAVFPPAHQ | |
| | | | | QRT | |
| 413 | TSESQLFNK | late protein | 923 | MTEYKLVVVGA | |
| | | | | VGVGKSALTIQL | |
| | | | | - | |
| 414 | YYYAGSSRL | late protein | 924 | NTVFGAERKKR | |
| | | | | LFIIGPTSRDRS | |
| | | | | SP | |
| 415 | LQFIFQLCK | late protein | 925 | PAIRVPPVIPLG | |
| | | | | SRELFCSKLRR | |
| | | | | AA | |
| 416 | MTLCAEVKK | late protein | 926 | PGISSQHFTYQ | |
| | | | | GGVGGSWPVC | |
| | | | | SGLG | |
| 417 | QYRVFRIKL | late protein | 927 | PTPSAPCPATP | |
| | | | | AVPKGRVFVSP | |
| | | | | LAK | |
| 418 | YLLEMLWRL | Latent membrane protein | 928 | SFVNDIFERIAG | |
| | | 1 | | VASRLAHYNKR | |
| | | | | ST | |
| 419 | YLQQNWWTL | latent membrane protein 1 | 929 | SLAALKKALAD | |
| | | | | GGYDVEKNNS | |
| | | | | RI | |
| 420 | ALLVLYSFA | latent membrane | 930 | VISAEKAYHEQL | |
| | | protein 1 | | TVAEITNACFEP | |
| | | | | A | |
| 421 | CLGGLLTMV | Latent membrane | 931 | VRLAQGLTHLG | |
| | | protein | | KATLTLCPYHS | |
| | | 2 | | DRQ | |
| 422 | GLGTLGAAL | latent membrane | 932 | FISNTVFRK | |
| | | protein 2 | | | |
| 423 | LLSAWILTA | Latent membrane | 933 | FLFELIPEP | |
| | | protein | | | |
| | | 2 | | | |
| 424 | LLWTLVVLL | latent membrane | 934 | FLGEAWAQV | |
| | | protein 2 | | | |
| 425 | PYLFWLAAI | Latent membrane | 935 | FVGALSFSI | |
| | | protein | | | |
| | | 2 | | | |
| 426 | MGSLEMVPM | Latent membrane | 936 | ILGIFNEFV | |
| | | protein | | | |
| | | 2 | | | |
| 427 | SLGGLLTMA | Latent membrane | 937 | ILSPSAHEL | |
| | | protein | | | |
| | | 2 | | | |
| 428 | TYGPVFMSL | latent membrane | 938 | IMSSSLFNL | |
| | | protein | | | |
| | | 2A | | | |
| 429 | SSCSSCPLSKV | latent membrane | 939 | KIYRRQIFK | |
| | | protein | | | |
| | | 2A | | | |
| 430 | QKEKSLEFTKELPGY | leucine-rich repeat- | 940 | KLADYLNVL | |
| | | containing | | | |
| | | protein 37A3 | | | |
| | | isoform 1 precursor | | | |
| | | [Homo | | | |
| | | sapiens] | | | |
| 431 | PGESLRPRGERRLPQ | leukocyte | 941 | KVFEHVGSR | |
| | | immunoglobulin | | | |
| | | like receptor A6 | | | |
| 432 | PGSGPQNRLGRYLEV | leukocyte | 942 | LLHGFSFYL | |
| | | immunoglobulin- | | | |
| | | like receptor | | | |
| | | subfamily B | | | |
| | | member 3 isoform X2 | | | |
| | | [Homo sapiens] | | | |
| 433 | SSFGRGFFK | liprin-beta1 | 943 | LLVDLAEEL | |
| 434 | AFFLDLILLIIALYL | LMP1 protein | 944 | LPRAKKLIL | |
| | QQNWW | (Epstein- | | | |
| | | Barr virus, | | | |
| | | putative | | | |
| | | LYDMA gene) | | | |
| 435 | ALLVLYSFALMLII | LMP1 protein | 945 | NLRYFAKSL | |
| | IILIIF | (Epstein- | | | |
| | | Barr virus, | | | |
| | | putative | | | |
| | | LYDMA gene) | | | |
| 436 | CLLVLGIWIYLLEMLW | LMP1 protein | 946 | QMIYSAARV | |
| | RLGA | (Epstein- | | | |
| | | Barr virus, | | | |
| | | putative | | | |
| | | LYDMA gene) | | | |
| 437 | DWTGGALLVLYSFAL | LMP1 protein | 947 | RLFGEAPREL | |
| | MLIII | (Epstein- | | | |
| | | Barr virus, | | | |
| | | putative | | | |
| | | LYDMA gene) | | | |
| 438 | GALCILLLMITLLLIAL | LMP1 protein | 948 | RLSDFSEQL | |
| | WNL | (Epstein- | | | |
| | | Barr virus, | | | |
| | | putative | | | |
| | | LYDMA gene) | | | |
| 439 | GIWIYLLEMLWRLGAT | LMP1 protein | 949 | RMWDFDIFL | |
| | IWQL | (Epstein- | | | |
| | | Barr virus, | | | |
| | | putative | | | |
| | | LYDMA gene) | | | |
| 440 | GPRRPPRGPPLSSSL | LMP1 protein | 950 | SLLRSLENV | |
| | GLALL | (Epstein- | | | |
| | | Barr virus, | | | |
| | | putative | | | |
| | | LYDMA gene) | | | |
| 441 | ILIIFIFRRDLLCPL | LMP1 protein | 951 | SLRSHHYSL | |
| | GALCI | (Epstein- | | | |
| | | Barr virus, | | | |
| | | putative | | | |
| | | LYDMA gene) | | | |
| 442 | LFLAILIWMYYHGQRH | LMP1 protein | 952 | VLDGFIPGT | |
| | SDEH | (Epstein- | | | |
| | | Barr virus, | | | |
| | | putative | | | |
| | | LYDMA gene) | | | |
| 443 | LILLIIALYLQQNWW | LMP1 protein | 953 | VLQEATICV | |
| | TLLVD | (Epstein- | | | |
| | | Barr virus, | | | |
| | | putative | | | |
| | | LYDMA gene) | | | |
| 444 | LLFWLYIVMSDWTGG | LMP1 protein | 954 | WVLALFDEV | |
| | ALLVL | (Epstein- | | | |
| | | Barr virus, | | | |
| | | putative | | | |
| | | LYDMA gene) | | | |
| 445 | LLLMITLLLIALWNL | LMP1 protein | 955 | YILKYSVFL | |
| | HGQAL | (Epstein- | | | |
| | | Barr virus, | | | |
| | | putative | | | |
| | | LYDMA gene) | | | |
| 446 | LLWLLLFLAILIWMYY | LMP1 protein | 956 | DANSFLQSV | |
| | HGQR | (Epstein- | | | |
| | | Barr virus, | | | |
| | | putative | | | |
| | | LYDMA gene) | | | |
| 447 | LSSSLGLALLLLLL | LMP1 protein | 957 | TEYKLVVVGAV | |
| | ALLFWL | (Epstein- | | GVGKSALTIQ | |
| | | Barr virus, | | | |
| | | putative | | | |
| | | LYDMA gene) | | | |
| 448 | NDGGPPQLTEEVENK | LMP1 protein | 958 | AGAFLSSPGLL | |
| | GGDQG | (Epstein- | | AVFG | |
| | | Barr virus, | | | |
| | | putative | | | |
| | | LYDMA gene) | | | |
| 449 | PRGPPLSSSLGLALLL | LMP1 protein | 959 | ARGGLVDEKAL | |
| | LLLA | (Epstein- | | ARALKEGRIRG | |
| | | Barr virus, | | AAL | |
| | | putative | | | |
| | | LYDMA gene) | | | |
| 450 | QQNWWTLLVDLLWL | LMP1 protein | 960 | FEDKSVAYT | |
| | LLFLAI | (Epstein- | | | |
| | | Barr virus, | | | |
| | | putative | | | |
| | | LYDMA gene) | | | |
| 451 | YHGQRHSDEHHHDD | LMP1 protein | 961 | FYNDIILMV | |
| | SLPHPQ | (Epstein- | | | |
| | | Barr virus, | | | |
| | | putative | | | |
| | | LYDMA gene) | | | |
| 452 | YIVMSDWTGGALLVL | LMP1 protein | 962 | GFPAERLEGVY | |
| | YSFAL | (Epstein- | | SNNI | |
| | | Barr virus, | | | |
| | | putative | | | |
| | | LYDMA gene) | | | |
| 453 | YSFALMLIIIILI | LMP1 protein | 963 | GLNFVSVKGPE | |
| | IFIFRRD | (Epstein- | | LLNM | |
| | | Barr virus, | | | |
| | | putative | | | |
| | | LYDMA gene) | | | |
| 454 | PSHOPPASTLSPNPT | LOC285679 protein | 964 | KALARALKEGRI | |
| | | [Homo sapiens] | | R | |
| 455 | RYCNLEGPPI | LY6K protein, | 965 | LILFFHTLGLQT | |
| | | partial | | KEE | |
| | | [Homo sapiens] | | | |
| 456 | YMASWVMLGITYRNK | lymphocyte | 966 | NNTDIRLIGEKL | |
| | SLMW | antigen 75 | | FHG | |
| | | precursor | | | |
| 457 | SPRPPLGSSL | lysine-specific | 967 | RYYVGHKAKF | |
| | | demethylase | | | |
| | | 2B isoform a | | | |
| 458 | DEMDCPLSPTPPLCS | MAM and LDL- | 968 | YEGQVISNGF | |
| | | receptor | | | |
| | | class A domain- | | | |
| | | containing | | | |
| | | protein 1 | | | |
| | | isoform X7 | | | |
| | | [Homo sapiens] | | | |
| 459 | FLNQTDETL | Mammaglobin-A | 969 | CLWGRLWQL | |
| | | precursor | | | |
| 460 | KLLMVLMLA | Mammaglobin-A | 970 | ALPLSEPMRRS | |
| | | precursor | | VSEE | |
| 461 | LIYDSSLCDL | Mammaglobin-A | 971 | EMSALLARRRR | |
| | | precursor | | IVEK | |
| 462 | LMVLMLAAL | Mammaglobin-A | 972 | FLQEVNVCGV | |
| | | precursor | | | |
| 463 | TINPQVSKT | Mammaglobin-A | 973 | KLVVVGADGV | |
| | | precursor | | | |
| 464 | LWHLQGPKDLMLKLR | matriptase-2 | 974 | LKLMAYLAGAK | |
| | | [Homo | | YTGC | |
| | | sapiens] | | | |
| 465 | YLEKESIYY | Matrix protein | 975 | LLKAELVAGL | |
| 466 | GILGFVFTL | Matrix protein 1 | 976 | PKPVLHMVSSE | |
| | | | | QHSG | |
| 467 | RLEDVFAGK | Matrix protein 1 | 977 | QWINYFDKRRD | |
| | | | | YLKF | |
| 468 | VMMHGGPPHPGMP | Meis1, myeloid | 978 | RTDLVKSELLHI | |
| | MS | ecotropic | | ECQ | |
| | | viral integration | | | |
| | | site 1 | | | |
| | | homolog (mouse), | | | |
| | | isoform | | | |
| | | CRA_e [Homo sapiens] | | | |
| 469 | ALLAVGATK | Melanocyte protein Pmel | 979 | SEIVPCLSERHK | |
| | | 17 precursor | | AYL | |
| 470 | ITDQVPFSV | Melanocyte protein Pmel | 980 | SFYNNLVSFAS | |
| | | 17 precursor | | PLVT | |
| 471 | WNRQLYPEWTEAQR | Melanocyte protein Pmel | 981 | VAGCYLKYKNK | |
| | LD | 17 precursor | | NSLS | |
| 472 | YLEPGPVTA | Melanocyte protein Pmel | 982 | AFMYAKKGEW | |
| | | 17 precursor | | KKAEE | |
| 473 | NRQLYPEWTEAQRL | Melanocyte protein Pmel | 983 | AFTMLLYCELL | |
| | D | 17 precursor | | QWED | |
| 474 | NRQLYPEWTEAQR | Melanocyte protein Pmel | 984 | AGQNPASDPPP | |
| | | 17 precursor | | DDAE | |
| 475 | RTKAWNRQLYPEWT | Melanocyte protein Pmel | 985 | AGRFGQGDHH | |
| | EAQR | 17 precursor | | AAGQA | |
| 476 | WNRQLYPEWTEAQR | Melanocyte protein Pmel | 986 | ALKIKGIRPYHS | |
| | | 17 precursor | | LSY | |
| 477 | RYGSFSVTL | Melanocyte protein Pmel | 987 | ARVILGVCWYV | |
| | | 17 precursor | | ETTS | |
| 478 | ASSIMSTESITGSLGP | Melanocyte protein Pmel | 988 | AVKRLPLIYCDY | |
| | LLDG | 17 precursor | | HGH | |
| 479 | ESITGSLGPLL | Melanocyte protein Pmel | 989 | AVVFQDSMVFR | |
| | | 17 precursor | | VAPW | |
| 480 | HRRGSRSYV | Melanocyte protein Pmel | 990 | AVYTPPSDSTH | |
| | | 17 precursor | | QMPR | |
| 481 | STESITGSLGPLLDGT | Melanocyte protein Pmel | 991 | CNECGKALCQS | |
| | ATLR | 17 precursor | | PSLI | |
| 482 | THTMEVTVYHRRGSR | Melanocyte protein Pmel | 992 | CNFSTIDVSLKT | |
| | SYVPL | 17 precursor | | DTE | |
| 483 | VLYRYGSFSVTLDIVQ | Melanocyte protein Pmel | 993 | CRGSGKSKVGT | |
| | GIES | 17 precursor | | SGDH | |
| 484 | VPLDCVLYRYGSFSV | Melanocyte protein Pmel | 994 | CVSMLGVLVDP | |
| | TLDIV | 17 precursor | | DTLH | |
| 485 | VTVYHRRGSRSYVPL | Melanocyte protein Pmel | 995 | DEMDCPLRPTP | |
| | AHSSS | 17 precursor | | PLCS | |
| 486 | AMLGTHTMEV | melanoma antigen gp100 | 996 | DFGFALTLAAP | |
| | | | | GDI | |
| 487 | ALYVDSLFFL | melanoma antigen | 997 | DPSAIGLVDPPI | |
| | | preferentially expressed in | | PSP | |
| | | tumors | | | |
| 488 | SQLTTLSFY | melanoma antigen | 998 | ECGKAFNSSSN | |
| | | preferentially expressed in | | LTKH | |
| | | tumors | | | |
| 489 | AAGIGILTV | Melanoma antigen | 999 | ECGQAFSISSN | |
| | | recognized by T cells 1 | | LMRH | |
| 490 | EAAGIGILTV | Melanoma antigen | 1000 | EECGKAFRVFS | |
| | | recognized by T cells 1 | | TLTK | |
| 491 | ILTVILGVL | Melanoma antigen | 1001 | EECGKPFKRFS | |
| | | recognized by T cells 1 | | YLTV | |
| 492 | RNGYRALMDKSLHV | Melanoma antigen | 1002 | EFPVLQAAAIYL | |
| | GTQCALTRR | recognized by T cells 1 | | K | |
| 493 | APPAYEKLSAEQ + | Melanoma antigen | 1003 | EHSQETEILREA | |
| | PHOS(S9) | recognized by T cells 1 | | LLS | |
| 494 | APPAYEKLSAEQSPP | Melanoma antigen | 1004 | EKKQQFRSLKE | |
| | + PHOS(S9) | recognized by T cells 1 | | KCFL | |
| 495 | APPAYEKLSAEQSPP | Melanoma antigen | 1005 | EKPYSCPECSL | |
| | P + PHOS(S9) | recognized by T cells 1 | | RFAY | |
| 496 | APPAYEKLSAEQSPP | Melanoma antigen | 1006 | EPGDTALHLCA | |
| | PY + PHOS(S9) | recognized by T cells 1 | | SSQS | |
| 497 | NAPPAYEKLSAE | Melanoma antigen | 1007 | FASPGDDRDG | |
| | PHOS(S10) | recognized by T cells 1 | | RAEGF | |
| 498 | VPNAPPAYEKLSAEQ | Melanoma antigen | 1008 | FEGTEMWYPN | |
| | SPPPY + PHOS | recognized by T cells 1 | | RELSE | |
| | (S12) | | | | |
| 499 | EEAAGIGIL | Melanoma antigen | 1009 | FSISQLQTNHD | |
| | | recognized by T cells 1 | | MNDE | |
| 500 | GHGHSYTTAEEAAGI | Melanoma antigen | 1010 | GAIVIELPNVKW | |
| | GILTV | recognized by T cells 1 | | S | |
| 501 | YTTAEEAAGIGILT | Melanoma antigen | 1011 | GCLGGENCFRL | |
| | VILGVL | recognized by T cells 1 | | RLES | |
| 502 | EADPTGHSY | Melanoma-associated | 1012 | GEPIPQPVRLR | |
| | | antigen 1 | | YVTS | |
| 503 | RVRFFFPSL | Melanoma-associated | 1013 | GLLRYWRTERL | |
| | | antigen 1 | | F | |
| 504 | EVDPIGHLY | Melanoma-associated | 1014 | GLPTDTICKEFR | |
| | | antigen 3 | | TRM | |
| 505 | FLWGPRALV | Melanoma-associated | 1015 | GRKFAAWGPP | |
| | | antigen 3 | | SFSQT | |
| 506 | MEVDPIGHLY | Melanoma-associated | 1016 | GRLILWEGPPL | |
| | | antigen 3 | | GAGG | |
| 507 | EVDPIGHVY | melanoma-associated | 1017 | GRNSFEVLVCA | |
| | | antigen 6 | | CPGR | |
| 508 | LLFGLALIEV | Melanoma-associated | 1018 | GYGEMGSVYR | |
| | | antigen C2 | | EDLGA | |
| 509 | CPLSKILL | membrane protein | 1019 | HGLSHSLWQIS | |
| | | | | SQLS | |
| 510 | FLYALALL | membrane protein | 1020 | HKRIHNGDKPY | |
| | | | | KCEE | |
| 511 | FLYALALLL | membrane protein | 1021 | HNNIVYNKYISH | |
| | | | | REH | |
| 512 | GGSILQTNFKSLSS | membrane protein | 1022 | HQRTHTGDKPF | |
| | TEF | | | KCDE | |
| 513 | IEDPPFNSL | membrane protein | 1023 | IHKMIHTVEKPY | |
| | | | | KCE | |
| 514 | LLWTLVVL | membrane protein | 1024 | IHSSWDCSLFT | |
| | | | | NYSA | |
| 515 | LTAGFLIFL | membrane protein | 1025 | ILFSLQPGLLRY | |
| | | | | W | |
| 516 | PYLFWLAA | membrane protein | 1026 | ILLIHCDTHLHTP | |
| | | | | MY | |
| 517 | RRRWRRLTV | membrane protein | 1027 | IMASKGMHHFC | |
| | | | | LISE | |
| 518 | SSCSSCPLSK | membrane protein | 1028 | KAFSQSSSLRK | |
| | | | | HEII | |
| 519 | SSCSSCPLSKI | membrane protein | 1029 | KCDECGNDFN | |
| | | | | WPATL | |
| 520 | TYGPVFMCL | membrane protein | 1030 | KCEECDTDFSR | |
| | | | | KSHH | |
| 521 | VMSNTLLSAW | membrane protein | 1031 | KELRALREMVS | |
| | | | | NMSG | |
| 522 | VTLAHAGYY | membrane protein BILF2 | 1032 | KLKKKQVKVFA | |
| | | [Human | | | |
| | | gammaherpesvirus 4] | | | |
| 523 | PFSPSHPAPPSDPSH | membrane-associated | 1033 | KLSVAPSVVLE | |
| | | guanylate kinase, WW | | EDQV | |
| | | and PDZ domain- | | | |
| | | containing protein 2 | | | |
| | | isoform X11 [Homo | | | |
| | | sapiens] | | | |
| 524 | ALGIGVYPV | Membrane-associated | 1034 | KLVVVGAVGVG | |
| | | phosphatidylinositol | | KSAL | |
| | | transfer protein 1 | | | |
| 525 | VNTTTSPVNTTTSPV | membrane-spanning 4- | 1035 | KPVILGVCWYV | |
| | | domains subfamily A | | ETTS | |
| | | member 18 [Homo | | | |
| | | sapiens] | | | |
| 526 | RTDLVKSELLHIESQ | Methionine synthase | 1036 | KSEGEEAHEVE | |
| | | reductase | | GETQ | |
| 527 | ALGLGLLPV | MFS transporter | 1037 | LADGEGGATDE | |
| | | [bacterium JGI 053] | | GIYD | |
| 528 | DILEQARAAVDTYCR | MHC class II antigen, | 1038 | LELINKLLSPVV | |
| | | partial [Homo sapiens] | | PQ | |
| 529 | HSLRYFRLGVSDPIR | MHC class I-like antigen | 1039 | LFGLGKDVGW | |
| | GVPE | MR-1 | | GPPAR | |
| 530 | KDQGPIVPAPVKGEG | microtubule-associated | 1040 | LGLWRGEAVTL | |
| | | protein 6 isoform X4 | | SNPK | |
| | | [Homo sapiens] | | | |
| 531 | VKDQGPMVSAPVKD | microtubule-associated | 1041 | LLDRGSFWND | |
| | Q | protein 6 isoform X4 | | GLKAS | |
| | | [Homo sapiens] | | | |
| 532 | RASHPIVQK | midasin | 1042 | LLIHCDAYLHTP | |
| | | | | MYF | |
| 533 | LLSAVLPSV | Mini-chromosome | 1043 | LNKVTIDAIHRL | |
| | | maintenance complex- | | PL | |
| | | binding protein | | | |
| 534 | AQGWSTVARFQITAT | mitochondrial ATP-Mg/Pi | 1044 | LQNHIQTFHREL | |
| | | carrier protein, partial | | VPD | |
| | | [Homo sapiens] | | | |
| 535 | NTVFGAERKKRLSIIG | Mitogen-activated protein | 1045 | LRPQLAEKKQQ | |
| | PTSRDRSSP | kinase kinase kinase 2 | | FRNL | |
| 536 | MSYDYHQNWGRDG | MLEL1 protein [Homo | 1046 | LTDHRAHCCPG | |
| | G | sapiens] | | GNAK | |
| 537 | LNKVTIDARHRLPL | MORC family CW-type | 1047 | LWHLQGPEDL | |
| | | zinc finger protein 1 | | MLKLR | |
| | | [Homo sapiens] | | | |
| 538 | HGNSSIIADQIALKLV | MTHFD1 protein | 1048 | LWTEGMLKMAF | |
| | GPE | | | HILA | |
| 539 | TTTASTEGSETTTAS | MUC22, partial [Homo | 1049 | MNAAVTFTNCA | |
| | | sapiens] | | LGRV | |
| 540 | STAPPVHNV | Mucin-1 | 1050 | MSYDYHHNWG | |
| | | | | RDGG | |
| 541 | LLLLTVLTV | Mucin-1 precursor | 1051 | NLVHGPPGPPQ | |
| | | | | VGAD | |
| 542 | FASPGDDGDGRAEG | Mucin-12 | 1052 | NVSFFHYQEYG | |
| | F | | | Y | |
| 543 | LAGIGLIAA | MULTISPECIES: gamma- | 1053 | PFSPSHPGPPS | |
| | | glutamyltransferase | | DPSH | |
| | | [Pseudomonas] | | | |
| 544 | LGGLGLFFA | Mycocerosic acid | 1054 | PMEKPTITTEKP | |
| | | synthase | | TIP | |
| 545 | VLQELNVTV | Myeloblastin precursor | 1055 | PRCCISSFCRP | |
| | | | | SCCV | |
| 546 | EHSQETESLREALLS | myomegalin isoform X22 | 1056 | PSRHRYGTRQP | |
| | | [Homo sapiens] | | RARL | |
| 547 | YIGEVLVSV | myosin IG | 1057 | PTKCEVEQFTA | |
| | | | | TSFG | |
| 548 | FLIIILDHL | Myotubularin | 1058 | PVCSGASSSCC | |
| | | | | QQSS | |
| 549 | KLSVAPSEVLEEDQV | N-acetyllactosaminide | 1059 | PWRKFPVHVLG | |
| | | alpha-1,3- | | QFLG | |
| | | galactosyltransferase | | | |
| 550 | NQLKERSFAQLISKD | NACHT, LRR and PYD | 1060 | QDLMLEDNLLK | |
| | | domains-containing | | LEV | |
| | | protein 14 [Homo sapiens] | | | |
| 551 | YVSMMCNEQAYSLA | NADH dehydrogenase | 1061 | QEVEGETHKTE | |
| | V | [ubiquinone]iron-sulfur | | GDAQ | |
| | | protein 2, mitochondrial | | | |
| | | isoform 1 precursor [Homo | | | |
| | | sapiens] | | | |
| 552 | FLKGIGWIPI | Nebulin | 1062 | QLLEGLGCTLT | |
| | | | | VVPE | |
| 553 | LRPQLAENKQQFRNL | neuroblastoma breakpoint | 1063 | QLTAHKMNHTG | |
| | | family member 11 isoform | | EKPY | |
| | | b [Homo sapiens] | | | |
| 554 | EKKQQFRNLKEKCFL | neuroblastoma breakpoint | 1064 | RKFISLHKKALE | |
| | | family member 26 [Homo | | SDF | |
| | | sapiens] | | | |
| 555 | KALRLSASALF | neurosecretory protein | 1065 | SGSGSGPFPSL | |
| | | VGF precursor | | FLNS | |
| 556 | AFMYAKKEEWKKAEE | Neutrophil cytosolic factor | 1066 | SHNSSLIFHQR | |
| | | 2 [Homo sapiens] | | VHTG | |
| 557 | TMLDIQPED | Non-structural protein 2a | 1067 | SKMGKWCSHC | |
| | | | | FAWCR | |
| 558 | GVFVYGGSKTSLYNL | nuclear antigen EBNA-1 | 1068 | SKMGKWCSHC | |
| | | | | FPCCR | |
| 559 | FLRGRAYGL | nuclear antigen EBNA-3 | 1069 | SNDSSLTHHQR | |
| | | | | VHTG | |
| 560 | KLKKKQVNVFA | nuclear receptor co- | 1070 | SNLTKHKIIHIEK | |
| | | repressor 1, isoform | | KP | |
| | | CRA_c [Homo sapiens] | | | |
| 561 | ILRGSVAHK | Nucleoprotein | 1071 | SNLTTHKIIHTG | |
| | | | | ERP | |
| 562 | VYGIRLEHF | NUF2R | 1072 | SSGGGSSSGG | |
| | | | | YGGGS | |
| 563 | SLLMWITQCFLPVF | NY-ESO-1 protein | 1073 | SSLPGPPGPPG | |
| | | | | PRGY | |
| 564 | YLAMPFATPMEAELA | NY-ESO-1 protein | 1074 | STAYPAPVRRR | |
| | RRSLA | | | CCLP | |
| 565 | LAMPFATPM | NY-ESO-1 protein | 1075 | STLLTEHLRIHT | |
| | | | | GEK | |
| 566 | MPFATPMEA | NY-ESO-1 protein | 1076 | STLNTHKSIHTG | |
| | | | | EEP | |
| 567 | MPFATPMEAEL | NY-ESO-1 protein | 1077 | TCCRTTCFRPS | |
| | | | | CCVS | |
| 568 | PFATPMEAELAR | NY-ESO-1 protein | 1078 | TFNCHHAQPW | |
| | | | | HNQFV | |
| 569 | QDAPPLPVPGVLLKE | NY-ESO-1 protein | 1079 | TGAMNVAIGTIQ | |
| | FTVSGNILTIRLTAA | | | TGV | |
| | DHR | | | | |
| 570 | ILLIHCDAHLHTPMY | OR2T4, partial [Homo | 1080 | THRPGGKRGRL | |
| | | sapiens] | | AGGS | |
| 571 | LLIHCDAHLHTPMYF | OR2T4, partial [Homo | 1081 | TIYSLFYSVADQ | |
| | | sapiens] | | DAPA | |
| 572 | ACDPHSGHFV | orf | 1082 | TMLARLVLDS | |
| 573 | LFMDTLSFVCPLC | ORF putative E7 protein | 1083 | TQLRLPGWPTP | |
| | | | | VSFG | |
| 574 | IGLITVLFL | ORF28 | 1084 | TRLFPNELANF | |
| | | | | YNAV | |
| 575 | GRNSFEVRVCACPG | p53 transformation | 1085 | TSCARRDYPRA | |
| | R | suppressor, partial [Homo | | SSPN | |
| | | sapiens] | | | |
| 576 | GLAPPQHLIRV | P53_HUMAN Cellular | 1086 | VMMHGGPAHP | |
| | | tumor antigen p53 (Tumor | | GMPMS | |
| | | suppressor p53) | | | |
| | | (Phosphoprotein p53) | | | |
| | | (Antigen NY-CO-13) | | | |
| 577 | KTCPVQLWV | P53_HUMAN Cellular | 1087 | VNTTTSPANTT | |
| | | tumor antigen p53 (Tumor | | TSPV | |
| | | suppressor p53) | | | |
| | | (Phosphoprotein p53) | | | |
| | | (Antigen NY-CO-13) | | | |
| 578 | LLGRNSFEV | P53_HUMAN Cellular | 1088 | VPVAQVTMTST | |
| | | tumor antigen p53 (Tumor | | TDAD | |
| | | suppressor p53) | | | |
| | | (Phosphoprotein p53) | | | |
| | | (Antigen NY-CO-13) | | | |
| 579 | RMPEAAPPV | P53_HUMAN Cellular | 1089 | VQLRGRALGG | |
| | | tumor antigen p53 (Tumor | | GALRA | |
| | | suppressor p53) | | | |
| | | (Phosphoprotein p53) | | | |
| | | (Antigen NY-CO-13) | | | |
| 580 | YQGSYGFRL | P53_HUMAN Cellular | 1090 | VRRCLPLWALT | |
| | | tumor antigen p53 (Tumor | | LEAA | |
| | | suppressor p53) | | | |
| | | (Phosphoprotein p53) | | | |
| | | (Antigen NY-CO-13) | | | |
| 581 | YLLPAIVHI | p68 RNA helicase | 1091 | VTGGRGGWQG | |
| | | | | PSPAF | |
| 582 | QLLDGFMITL | PAS domain containing | 1092 | VTVHPTSKSTA | |
| | | protein 1 | | TSQG | |
| 583 | YLVGNVCIL | PAS domain containing | 1093 | YVSMMCNKQA | |
| | | protein 1 | | YSLAV | |
| 584 | STMPHTSGMNR | PAX-3-FKHR gene fusion, | 1094 | FYGKTILWF | |
| | | partial | | | |
| 585 | GRKFAAWAPPSFSQT | pentraxin 4 | 1095 | CLGQLSNA | |
| 586 | GMMRWCMPV | peptidase, U32 family | 1096 | GDRFCLGQLSN | |
| | | | | AHRT | |
| 587 | AVVFQDSVVFRVAPW | peptidyl arginine | 1097 | VYFFLPDHL | |
| | | deiminase, type IV [Homo | | | |
| | | sapiens] | | | |
| 588 | TSALPIIQK | Perilipin-2 | 1098 | AVMRWGMPL | |
| 589 | DQYPYLKSV | Perilipin-2 | 1099 | KVDPIGHVY | |
| 590 | IARNLTQQL | Perilipin-2 | 1100 | VMLEGEQEA | |
| 591 | SLLTSSKGQLQK | Perilipin-2 | 1101 | VQLEEEYEV | |
| 592 | TGAMNVAKGTIQTGV | perilipin-4 isoform X1 | 1102 | ETMQCSELYHM | |
| | | [Homo sapiens] | | | |
| 593 | ATAGDGLIELRK | PHB, partial | 1103 | EVIVPLSGW | |
| 594 | GSSDVIIHR | PHD and ring finger | 1104 | EVQQFLRY | |
| | | domains 1 | | | |
| 595 | PYGCLPTGDRTGLIE | phosphatidylinositol 4,5- | 1105 | QHQPNPFEV | |
| | | bisphosphate 3-kinase | | | |
| | | catalytic subunit delta | | | |
| | | isoform isoform 2 [Homo | | | |
| | | sapiens] | | | |
| 596 | SRSSSAELDRSR | phosphatidylinositol 4- | 1106 | EFEFAQFPNWY | |
| | | kinase type 2-beta | | ISTS | |
| 597 | ALGIGIYSL | phosphatidylinositol-4,5- | 1107 | RMQAASFGTFE | |
| | | bisphosphate 3-kinase | | QWVV | |
| | | catalytic subunit | | | |
| 598 | KTWGQYWQV | Pmel 17 | 1108 | SGHLLLQKLLR | |
| | | | | AKNV | |
| 599 | SIFDGRVVAK | PNAS-136 | 1109 | ASMPSSPPL | |
| 600 | DLDVKKMPL | poly [ADP-ribose] | 1110 | DYMIHIIEKW | |
| | | polymerase 3 isoform a | | | |
| 601 | SKMGKWCRHCFPCC | POTE ankyrin domain | 1111 | FICAIIVVV | |
| | R | family member F isoform | | | |
| | | X1 [Homo sapiens] | | | |
| 602 | CRGSGKSNVGTSGD | POTE22 [Homo sapiens] | 1112 | FLGAGLFLYF | |
| | H | | | | |
| 603 | SKMGKWCRHCFAWC | POTE22 [Homo sapiens] | 1113 | GAQSWLWFV | |
| | R | | | | |
| 604 | TRATKMQVI | pp65 | 1114 | GRKLFGTHF | |
| 605 | VYALPLKML | pp65 | 1115 | IPINPRRCL | |
| 606 | YSEHPTFTSQY | pp65 | 1116 | KQWLVWLFL | |
| 607 | AVQEFGLARFK | PRELI | 1117 | LDYEWGTVTF | |
| 608 | GEPIPQPARLRYVTS | prickle-like protein 2 | 1118 | RVRVMAIYK | |
| | | [Homo sapiens] | | | |
| 609 | EMQEERLKLPILSEE | probable ATP-dependent | 1119 | SILEQMHRK | |
| | | RNA helicase DHX37 | | | |
| | | [Homo sapiens] | | | |
| 610 | VMLEGEQEE | Programmed cell death | 1120 | SMACVGFFL | |
| | | protein 7 | | | |
| 611 | HIGIGLLSL | proteasome (prosome, | 1121 | TSDYLSQSY | |
| | | macropain) activator | | | |
| | | subunit 4 | | | |
| 727 | SILEDPPSI | protein asunder homolog | 1122 | VADINDHAL | |
| | | | 1123 | VYRPLHYPLL | |

In some embodiments, the methods and compositions of the disclosure are used in combination with Kymriah™ (tisagenlecleucel; Novartis) suspension for intravenous infusion, formerly CTL019.

Suitable antigens are known in the art and are available from commercial, government, and scientific sources. The antigens may be purified or partially purified polypeptides derived from tumors. The antigens can be recombinant polypeptides produced by expressing DNA encoding the polypeptide antigen in a heterologous expression system. The DNA may be in the form of vector DNA such as plasmid DNA.

In certain embodiments, antigens may be provided as single antigens or may be provided in combination. Antigens may also be provided as complex mixtures of polypeptides or nucleic acids.

### Polar Block/Linker

For the conjugate to be trafficked efficiently to the lymph node, the conjugate should remain soluble at the injection site. Therefore, a polar block linker can be included between the cargo and the lipid to increase solubility of the conjugate. The polar block can also reduce or prevent the ability of cargo, such as a peptide, from non-specifically associating with extracellular matrix proteins at the site of administration. The polar block increases the solubility of the conjugate without preventing its ability to bind to albumin. It is believed that this combination of characteristics allows the conjugate to bind to albumin present in the serum or interstitial fluid, and remain in circulation until the albumin is trafficked to, and retained in a lymph node.

The length and composition of the polar block can be adjusted based on the lipid and cargo selected.

A polar block can be used as part of any of lipid conjugates suitable for use in the methods disclosed herein, for example, amphiphilic oligonucleotide conjugates and amphiphilic ligand conjugates, which reduce cell membrane insertion/preferential portioning on albumin. Suitable polar blocks include, but are not limited to, oligonucleotides such as those discussed above, a hydrophilic polymer including but not limited to poly(ethylene glycol) (MW: 500 Da to 20,000 Da), polyacrylamide (MW: 500 Da to 20,000 Da), polyacrylic acid; a string of hydrophilic amino acids such as serine, threonine, cysteine, tyrosine, asparagine, glutamine, aspartic acid, glutamic acid, lysine, arginine, histidine, or combinations thereof polysaccharides, including but not limited to, dextran (MW: 1,000 Da to 2,000,000 Da), or combinations thereof.

The hydrophobic lipid and the linker/cargo are covalently linked. The covalent bond may be a non-cleavable linkage or a cleavable linkage. The non-cleavable linkage can include an amide bond or phosphate bond, and the cleavable linkage can include a disulfide bond, acid-cleavable linkage, ester bond, anhydride bond, biodegradable bond, or enzyme-cleavable linkage.

### Ethylene Glycol Linkers

In certain embodiments, the polar block is one or more ethylene glycol (EG) units, more preferably two or more EG units (i.e., polyethylene glycol (PEG)). For example, in certain embodiments, a lipid conjugate includes a protein or peptide (e.g., peptide antigen) and a hydrophobic lipid linked by a polyethylene glycol (PEG) molecule or a derivative or analog thereof.

In certain embodiments, protein conjugates suitable for use in the methods disclosed herein contain protein antigen linked to PEG which is in turn linked to a hydrophobic lipid, or lipid-Gn-ON conjugates, either covalently or via formation of protein-oligo conjugates that hybridize to oligo micelles.

The precise number of EG units depends on the lipid and the cargo, however, typically, a polar block can have between about 1 and about 100, between about 20 and about 80, between about 30 and about 70, or between about 40 and about 60 EG units. In certain embodiments, the polar block has between about 45 and 55 EG, units. For example, in certain embodiments, the polar block has 48 EG units. In some embodiments, the EG units are consecutive (e.g., 24 consecutive EG units).

### T Cell Receptor (TCR)

In some aspects, the disclosure provides compositions and methods to be used or performed in in conjunction with TCR modified immune cells (e.g. T cells). Methods described herein include administering to a subject a composition including an amphiphilic ligand conjugate described herein and a T cell receptor modified immune cell. In some embodiments, the TCR binds the peptide of the amphiphilic ligand conjugate, Antigenic peptides bound to MHC molecules are presented to T cells by APC(s). Recognition and engagement of such peptide-MHC complex (pMHC) by the TCR, a molecule found on the surface of T cells, results in T cell activation and response. The TCR is a heterodimer composed of two different protein chains. In most T cells (about 95%), these two protein chains are alpha (α) and beta (β) chains. However, in a small percentage of T cells (about 5%), these two protein chains are gamma and delta (γ/δ) chains. The ratio of TCRs comprised of α/β chains versus γ/δ chains may change during a diseased state (e.g., in cancer (e.g., in a tumor), infectious disease, inflammatory disease or autoimmune disease). Engagement of the TCR with pMHC activates a T cell through a series of biochemical events mediated by associated enzymes, co-receptors, specialized adaptor molecules, and activated or released transcription factors.

Each of the two chains of a TCR contains multiple copies of gene segments—a variable ‘V’ gene segment, a diversity ‘D’ gene segment, and a joining ‘J’ gene segment. The TCR alpha chain is generated by recombination of V and J segments, while the beta chain is generated by recombination of V, D, and J segments. Similarly, generation of the TCR gamma chain involves recombination of V and J gene segments, while generation of the TCR delta chain occurs by recombination of V, D, and J gene segments. The intersection of these specific regions (V and J for the alpha or gamma chain, or V, D and J for the beta or delta chain) corresponds to the CDR3 region that is important for antigen-MHC recognition. Complementarity determining regions (e.g., CDR1, CDR2, and CDR3), or hypervariable regions, are sequences in the variable domains of antigen receptors that can complement an antigen.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3γ chain, a CD3δ chain, and two CD3ε chains. These chains associate with a T cell receptor (TCR) and CD3ζ to generate an activation signal in T lymphocytes. A complete TCR complex includes a TCR, CD3ζ, and the complete CD3 complex.

Any immune cell may be modified with a TCR. For example, the immune cell modified with a TCR described herein may be a T cell, a B cell, a natural killer (NK) cell, a macrophage, a neutrophil, a dendritic cell, a mast cell, an eosinophil, or a basophil. In particular embodiments, the immune cell modified with a TCR is a T cell.

### Modified TCR Immune Cells

Engineered immune cell therapy is used to generate immune cells modified with TCRs that are capable of recognizing a tumor in a subject. A TCR may recognize a tumor by the antigen present on the major histocompatibility complex (MHC) the tumor cell surface. In some embodiments, immune cells (e.g., T cells, B cells, NK cells, neutrophils, eosinophils, basophils, and granulocytes) are modified to express a TCR. In some embodiments, the immune cell is a mucosal-associated invariant T (MAIT) cell. In some embodiments, the immune cell is a human MAIT cell. MAIT cells are a population of T cells that display a semi-invariant TCR and are restricted by the evolutionarily conserved major histocompatibility complex class I-related protein, MR1. In some embodiments, T cells are modified to express a TCR. In some embodiments, the modified TCR may be used to activate and expand the immune cell (e.g., T cell) and/or increase proliferation of the immune cell (e.g., T cell). In some embodiments, activating and/or expanding the immune cell may be done in vitro. In some embodiments, activating and expanding the immune cell (e.g., T cell) may decrease the size of the tumor tissue or inhibit growth of the tumor cell population or tumor tissue in the subject.

TCR modified immune cells (e.g., T cells) may display desired specificities and enhanced functionalities. For example, TCR modified immune cells (e.g., T cells) may recognize a specific tumor-associated antigen. T cells can be genetically modified to express TCRs with altered specificity. The T cell may be modified with a TCR capable of recognizing a specific tumor-associated antigen. For example, in some embodiments, T cells are modified to express a modified TCR, where the TCR binds the peptide of the amphiphilic ligand conjugate. In some embodiments, binding of the peptide of the amphiphilic ligand conjugate allows for the activation and expansion of T cells directed towards a specific tumor.

An immune cell (e.g., T cell) may be modified with a TCR by introducing a recombinant nucleic acid encoding a TCR into a patient-derived T cell to generate a TCR modified immune cell (e.g., T cell). The modified T cell may then be administered back to the subject, for example, after being activated in vitro. In some embodiments, T cells not derived from the subject are genetically modified with a TCR. For example, in some embodiments, T cells are allogeneic cells that have been engineered to be used as an “off the shelf” adoptive cell therapy.

A variety of different methods known in the art can be used to introduce any of the nucleic acids or expression vectors disclosed herein into a T cell. Nonlimiting examples of methods for introducing nucleic acid into an immune cell (e.g., T cell) include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection. Furthermore, the CRISPR/Cas9 genome editing technology known in the art can be used to introduce nucleic acids into T cells

### Immunogenic Compositions

The conjugates suitable for use in the methods disclosed herein can be used in immunogenic compositions or as components in vaccines. Typically, immunogenic compositions disclosed herein include an amphiphilic lipid conjugate including a lipid, a peptide (e.g., a tumor associated antigen), optionally a linker, and/or a TCR modified immune cell (e.g., a TCR modified T cell), where the TCR binds the peptide of the amphiphilic ligand conjugate. The administration to a subject of both an amphiphilic lipid conjugate and a TCR modified immune cell is an example of a vaccine. When administered to a subject in combination, the amphiphilic lipid conjugate and the TCR modified immune cell can be administered in separate pharmaceutical compositions, or they can be administered together in the same pharmaceutical composition. Additionally, the vaccine may include an adjuvant. The adjuvant may be administered in the same pharmaceutical composition as the amphiphilic lipid conjugate and/or the TCR modified immune cell, or the adjuvant may be administered in a separate pharmaceutical composition.

### An Immunogenic Composition Suitable for Use in the Methods Disclosed Herein

An immunogenic composition suitable for use in the methods disclosed herein can include the combination of a composition including an amphiphilic ligand conjugate and a composition including a TCR modified immune cell (e.g., a TCR modified T cell). These compositions can be combined into one composition and can be administered alone, or in combination with an adjuvant. In some embodiments, the adjuvant is an amphiphilic oligonucleotide conjugate including an immunostimulatory oligonucleotide, as described supra.

The adjuvant may be, without limitation, alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene] (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include, without limitation, double-stranded RNA. Adjuvants that act through TLR4 include, without limitation, derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include, without limitation, flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG (Bacillus Colmette-Guérin); mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g., lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

### Methods of Making Polypeptides

In some embodiments, the peptides described herein for use in the amphiphilic conjugates (e.g., tumor associated antigens) are made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The methods of making polypeptides also include a vector capable of expressing the peptides in an appropriate host. The vector includes the DNA molecule that codes for the peptides operatively linked to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be suitable for use in the methods disclosed herein. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety, and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

The nucleic acid molecules described above can be contained within a vector that is capable of directing their expression in, for example, a cell that has been transduced with the vector. Accordingly, in addition to polypeptide mutants, expression vectors containing a nucleic acid molecule encoding a mutant and cells transfected with these vectors are among the certain embodiments.

Vectors suitable for use include T7-based vectors for use in bacteria (see, for example, Rosenberg et al., Gene 56: 125, 1987), the pMSXND expression vector for use in mammalian cells (Lee and Nathans, J. Biol. Chem. 263:3521, 1988), and baculovirus-derived vectors (for example the expression vector pBacPAKS from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neo^{r}) gene imparts G418 (Geneticin) resistance to cells in which it is expressed, and thus permits phenotypic selection of the transfected cells. Those of skill in the art can readily determine whether a given regulatory element or selectable marker is suitable for use in a particular experimental context.

Viral vectors that are suitable for use include, for example, retroviral, adenoviral, and adeno-associated vectors, herpes virus, simian virus 40 (SV 40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a polypeptide mutant are also suitable for use. A cell is a transfected cell, i.e., a cell into which a nucleic acid molecule, for example a nucleic acid molecule encoding a mutant polypeptide, has been introduced by means of recombinant DNA techniques. The progeny of such a cell are also considered suitable for use in the methods disclosed herein.

The precise components of the expression system are not critical. For example, a polypeptide mutant can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans of ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., conjugated to a lipid, as described herein.

### Pharmaceutical Composition and Modes of Administration

In some embodiments, an amphiphilic ligand conjugate and an immune cell modified to express a TCR are administered together (simultaneously or sequentially). In some embodiments, an amphiphilic ligand conjugate and an immune cell modified to express a modified TCR are administered together (simultaneously or sequentially). In some embodiments, the amphiphilic ligand conjugate including a lipid, a ligand of MAIT cell, and optionally a linker is administered to subject. In some embodiments, the amphiphilic ligand conjugate including a lipid, a ligand of MAIT cell is administered to the subject without an immune cell modified to express a TCR. In some embodiments, an amphiphilic ligand conjugate and an adjuvant (e.g., an amphiphilic oligonucleotide conjugate) are administered together (simultaneously or sequentially). In some embodiments, an amphiphilic ligand conjugate, an adjuvant (e.g., an amphiphilic oligonucleotide conjugate), and an immune cell modified to express a TCR are administered together (simultaneously or sequentially). In some embodiments, an amphiphilic ligand conjugate including a lipid, a ligand of MAIT cell, and optionally a linker, and an adjuvant (e.g., an amphiphilic oligonucleotide conjugate) are administered together (simultaneously or sequentially). In some embodiments, an amphiphilic ligand conjugate and an immune cell modified to express a TCR are administered separately. In some embodiments, an amphiphilic ligand conjugate and an adjuvant (e.g., an amphiphilic oligonucleotide conjugate) are administered separately. In some embodiments, an amphiphilic ligand conjugate, an adjuvant (e.g., an amphiphilic oligonucleotide conjugate) and an immune cell modified to express a TCR are administered separately.

In some embodiments, the disclosure provides for a pharmaceutical composition including an amphiphilic ligand conjugate with a pharmaceutically acceptable diluent, carrier, solubilizer, emulsifier, preservative and/or adjuvant. In some embodiments, the adjuvant is an amphiphilic oligonucleotide conjugate.

In some embodiments, acceptable formulation materials preferably are nontoxic to recipients at the dosages and concentrations employed. In certain embodiments, the formulation material(s) are for subcutaneous (s.c.) and/or intravenous (i.v.) administration. In some embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In some embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80 (polyoxyethylene (20) sorbitan monooleate), Triton X-100 (t-Octylphenoxypolyethoxyethanol), tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation includes PBS; 20 mM NaOAc, pH 5.2, 50 mM NaCl; and/or 10 mM NaOAc, pH 5.2, 9% Sucrose. In some embodiments, the optimal pharmaceutical composition will be determined by one skilled in the art depending upon, for example, the intended route of administration, delivery format and desired dosage. See, for example, Remington's Pharmaceutical Sciences, supra. In some embodiments, such compositions may influence the physical state, stability, rate of in vivo release and rate of in vivo clearance of the amphiphilic conjugate.

In some embodiments, the primary vehicle or carrier in a pharmaceutical composition can be either aqueous or non-aqueous in nature. For example, in some embodiments, a suitable vehicle or carrier can be water for injection, physiological saline solution or artificial cerebrospinal fluid, possibly supplemented with other materials common in compositions for parenteral administration. In some embodiments, the saline includes isotonic phosphate-buffered saline. In certain embodiments, neutral buffered saline or saline mixed with serum albumin are further exemplary vehicles. In some embodiments, pharmaceutical compositions include Tris buffer of about pH 7.0-8.5, or acetate buffer of about pH 4.0-5.5, which can further include sorbitol or a suitable substitute therefor. In some embodiments, a composition including an amphiphilic conjugate can be prepared for storage by mixing the selected composition having the desired degree of purity with optional formulation agents (Remington's Pharmaceutical Sciences, supra) in the form of a lyophilized cake or an aqueous solution. Further, in some embodiments, a composition including an amphiphilic conjugate, can be formulated as a lyophilizate using appropriate excipients such as sucrose.

In some embodiments, the pharmaceutical composition can be selected for parenteral delivery. In some embodiments, the compositions can be selected for inhalation or for delivery through the digestive tract, such as orally. The preparation of such pharmaceutically acceptable compositions is within the ability of one skilled in the art.

In some embodiments, the formulation components are present in concentrations that are acceptable to the site of administration. In some embodiments, buffers are used to maintain the composition at physiological pH or at a slightly lower pH, typically within a pH range of from about 5 to about 8.

In some embodiments, when parenteral administration is contemplated, a therapeutic composition can be in the form of a pyrogen-free, parenterally acceptable aqueous solution including an amphiphilic conjugate, in a pharmaceutically acceptable vehicle. In some embodiments, a vehicle for parenteral injection is sterile distilled water in which an amphiphilic conjugate is formulated as a sterile, isotonic solution, properly preserved. In some embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some embodiments, hyaluronic acid can also be used, and can have the effect of promoting sustained duration in circulation. In some embodiments, implantable drug delivery devices can be used to introduce the desired molecule.

In some embodiments, a pharmaceutical composition can be formulated for inhalation. In some embodiments, an amphiphilic conjugate can be formulated as a dry powder for inhalation. In some embodiments, an inhalation solution including an amphiphilic conjugate can be formulated with a propellant for aerosol delivery. In some embodiments, solutions can be nebulized. Pulmonary administration is further described in PCT Publication No. WO/1994/020069, which describes pulmonary delivery of chemically modified proteins.

In some embodiments, it is contemplated that formulations can be administered orally. In some embodiments, an amphiphilic conjugate that is administered in this fashion can be formulated with or without those carriers customarily used in the compounding of solid dosage forms such as tablets and capsules. In some embodiments, a capsule can be designed to release the active portion of the formulation at the point in the gastrointestinal tract when bioavailability is maximized and pre-systemic degradation is minimized. In some embodiments, at least one additional agent can be included to facilitate absorption of the amphiphilic conjugate. In certain embodiments, diluents, flavorings, low melting point waxes, vegetable oils, lubricants, suspending agents, tablet disintegrating agents, and binders can also be employed.

In some embodiments, a pharmaceutical composition can involve an effective quantity of an amphiphilic conjugate in a mixture with non-toxic excipients which are suitable for the manufacture of tablets. In some embodiments, by dissolving the tablets in sterile water, or another appropriate vehicle, solutions can be prepared in unit-dose form. In some embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled m the art, including formulations involving an amphiphilic conjugate in sustained- or controlled-delivery formulations. In some embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In some embodiments, sustained-release preparations can include semipermeable polymer matrices in the form of shaped articles, e.g., films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In some embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In some embodiments, this can be accomplished by filtration through sterile filtration membranes. In certain embodiments, where the composition is lyophilized, sterilization using this method can be conducted either prior to or following lyophilization and reconstitution. In some embodiments, the composition for parenteral administration can be stored in lyophilized form or in a solution. In some embodiments, parenteral compositions generally are placed into a container having a sterile access port, for example, an intravenous solution bag or vial having a stopper pierceable by a hypodermic injection needle.

In some embodiments, once the pharmaceutical composition has been formulated, it can be stored in sterile vials as a solution, suspension, gel, emulsion, solid, or as a dehydrated or lyophilized powder. In some embodiments, such formulations can be stored either in a ready-to-use form or in a form (e.g., lyophilized) that is reconstituted prior to administration.

In some embodiments, kits are provided for producing a single-dose administration unit. In some embodiments, the kit can contain both a first container having a dried protein and a second container having an aqueous formulation. In some embodiments, kits containing single and multi-chambered pre-filled syringes (e.g., liquid syringes and syringes containing a lyophilized therapeutic) are included.

In some embodiments, the effective amount of a pharmaceutical composition including an amphiphilic conjugate to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an amphiphilic conjugate is being used, the route of administration, and the size (body weight, body surface or organ size) and/or condition (the age and general health) of the patient. In some embodiments, the clinician can titer the dosage and modify the route of administration to obtain the optimal therapeutic effect.

In some embodiments, the frequency of dosing will take into account the pharmacokinetic parameters of the amphiphilic conjugate, in the formulation used. In some embodiments, a clinician will administer the composition until a dosage is reached that achieves the desired effect. In some embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In some embodiments, appropriate dosages can be ascertained through use of appropriate dose-response data.

In some embodiments, the route of administration of the pharmaceutical composition is in accord with known methods, e.g., orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes.

In some embodiments, the composition can be administered locally via implantation of a membrane, sponge or another appropriate material onto which the desired molecule has been absorbed or encapsulated. In some embodiments, where an implantation device is used, the device can be implanted into any suitable tissue or organ, and delivery of the desired molecule can be via diffusion, timed-release bolus, or continuous administration. In some embodiments, it can be desirable to use a pharmaceutical composition including an amphiphilic conjugate in an ex vivo manner. In such instances, cells, tissues and/or organs that have been removed from the patient are exposed to a pharmaceutical composition including an amphiphilic conjugate, after which the cells, tissues and/or organs are subsequently implanted back into the patient.

In some embodiments, an amphiphilic conjugate can be delivered by implanting certain cells that have been genetically engineered, using methods such as those described herein, to express and secrete the polypeptides. In some embodiments, such cells can be animal or human cells, and can be autologous, heterologous, or xenogeneic. In some embodiments, the cells can be immortalized. In some embodiments, in order to decrease the chance of an immunological response, the cells can be encapsulated to avoid infiltration of surrounding tissues. In some embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Methods

In some embodiments, the disclosure provides methods of expanding an immune cell expressing a T cell receptor in vivo in a subject, including administering a composition including an amphiphilic lipid conjugate described herein.

In some embodiments, the disclosure provides methods of stimulation proliferation of an immune cell expressing a T cell receptor in vivo in a subject, including administering a composition having an amphiphilic lipid conjugate described herein.

In some embodiments, the disclosure provides methods of activating, proliferating, phenotypically maturing, or inducing acquisition of cytotoxic function of a TCR T cell in vitro, including culturing the TCR T cell in the presence of a dendritic cell having an amphiphilic ligand conjugate described herein.

In some embodiments, the disclosure provides methods for treating a subject having a disease, disorder or condition associated with expression or elevated expression of an antigen, including administering to the subject an immune cell expressing a TCR targeted to the antigen, and an amphiphilic lipid conjugate.

In some embodiments, the subject is administered the immune cell expressing a TCR prior to receiving the amphiphilic lipid conjugate. In some embodiments, the subject is administered the immune cell expressing a TCR after receiving the amphiphilic lipid conjugate. In some embodiments, the subject is administered the immune cell expressing a TCR and the amphiphilic lipid conjugate sequentially or simultaneously.

In some embodiments, the disclosure provides a method of stimulating an immune response to a target cell population or target tissue in a subject including administering to the subject an amphiphilic ligand conjugate including a lipid, a ligand for a MAIT cell, and, optionally, a linker. In some embodiments, the method of stimulating an immune response to a target cell population or target tissue in a subject including administering to the subject an amphiphilic ligand conjugate including a lipid, a ligand for a MAIT cell, and, optionally, a linker, does not include administering an immune cell expressing a TCR to the subject. Conjugating a cargo, such as a peptide, to an albumin-binding domain can increase delivery and accumulation of the cargo to the lymph nodes, as described in U.S. Pat. No. 9,107,904 which is incorporated herein by reference in its entirety.

Methods for measuring expansion or proliferation of cells are known in the art. For example, the number of cells can be measured by introducing a dye (e.g., crystal violet) into cells, and measuring the dilution of the dye over time. Dilution indicates cell proliferation.

### Cancer and Cancer Immunotherapy

In some embodiments, the amphiphilic ligand conjugate and TCR modified immune cells (e.g., TCR modified T cells) described herein, are useful for treating a disorder associated with abnormal apoptosis or a differentiative process (e.g., cellular proliferative disorders (e.g., hyperproliferative disorders) or cellular differentiative disorders, such as cancer). Non-limiting examples of cancers that are amenable to treatment with the methods of the present invention are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast, bladder, rectum, stomach, skin, kidney, cervix, and liver. Accordingly, the compositions used herein including an amphiphilic ligand conjugate can be administered to a patient who has cancer.

As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, brain, stomach, liver, skin, thyroid, lymph glands and lymphoid tissue, gastrointestinal organs, and the genitourinary tract (e.g., bladder, kidney, and cervix), as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The amphiphilic ligand conjugate can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

It will be appreciated by those skilled in the art that amounts for an amphiphilic conjugate and TCR modified immune cells that are sufficient to reduce tumor growth and size, or a therapeutically effective amount, will vary not only on the particular compound or composition selected, but also with the route of administration, the nature of the condition being treated, and the age and condition of the patient, and will ultimately be at the discretion of the patient's physician or pharmacist. The length of time during which the compound used in the instant method will be given varies on an individual basis.

In some embodiments, the disclosure provides methods of reducing or decreasing the size of a tumor, or inhibiting a tumor growth in a subject in need thereof, including administering to the subject an amphiphilic lipid conjugate and a TCR modified T cell described herein to a subject. In some embodiments, the disclosure provides methods for inducing an anti-tumor response in a subject with cancer, including administering to the subject an amphiphilic lipid conjugate and a TCR modified immune cell described herein to a subject.

In some embodiments, the disclosure provides methods for stimulating an immune response to a target cell population or target tissue expressing an antigen in a subject, including administering an immune cell expressing a TCR targeted to the antigen, and an amphiphilic lipid conjugate. In some embodiments, the immune response is a T cell, a TIL (e.g., T cell, B cell, or an NK cell), an NK cell, an NKT cell, a gdT cell, a macrophage, a neutrophil, a dendritic cell, a mast cell, an eosinophil, or a basophil mediated immune response. In some embodiments, the immune response is an anti-tumor immune response. In some embodiments, the target cell population or target tissue is tumor cells or tumor tissue.

It will be appreciated by those skilled in the art that reference herein to treatment extends to prophylaxis as well as the treatment of the noted cancers and symptoms.

### Kits

A kit can include an amphiphilic ligand conjugate and a TCR modified immune cell (e.g., TCR modified T cells), as disclosed herein, and instructions for use. The kits may include, in a suitable container, an amphiphilic ligand conjugate, a TCR modified immune cell, one or more controls, and various buffers, reagents, enzymes and other standard ingredients well known in the art. In some embodiments, the kits further include an adjuvant. Accordingly, in some embodiments, the amphiphilic ligand conjugate and the TCR modified immune cell are in the same vial. In some embodiments, the amphiphilic ligand conjugate and the TCR modified immune cell are in separate vials. Furthermore, in some embodiments, the amphiphilic ligand conjugate and adjuvant are in the same vial. In some embodiments, the amphiphilic ligand conjugate and adjuvant are in separate vials. In some embodiments, the TCR immune cell and adjuvant are in the same vial. In some embodiments, the TCR immune cell and the adjuvant are in separate vials.

The container can include at least one vial, well, test tube, flask, bottle, syringe, or other container means, into which an amphiphilic ligand conjugate may be placed, and in some instances, suitably aliquoted. When an additional component is provided, the kit can contain additional containers into which this compound may be placed. The kits can also include a means for containing an amphiphilic ligand conjugate, a TCR modified immune cell, and any other reagent containers in close confinement for commercial sale. Such containers may include injection or blow-molded plastic containers into which the desired vials are retained. Containers and/or kits can include labeling with instructions for use and/or warnings.

In some embodiments, the disclosure provides a kit including a container including a composition including an amphiphilic ligand conjugate, a TCR modified immune cell (e.g., a TCR modified T cell), an optional pharmaceutically acceptable carrier, and a package insert including instructions for administration of the composition for treating or delaying progression of cancer in an individual receiving therapy with an immune cell expressing a T cell receptor, wherein the amphiphilic ligand conjugate includes a lipid, a peptide (e.g., a tumor associated antigen), and optionally a linker. In some embodiments, the kit further includes an adjuvant and instructions for administration of the adjuvant for treating or delaying progression of cancer in an individual receiving therapy with a TCR modified immune cell (e.g., a TCR modified T cell).

In some embodiments, the disclosure provides a kit including a medicament including a composition including an amphiphilic ligand conjugate, a TCR modified immune cell (e.g., a TCR modified T cell), an optional pharmaceutically acceptable carrier, and a package insert including instructions for administration of the medicament alone or in combination with a composition including an adjuvant and an optional pharmaceutically acceptable carrier, for treating or delaying progression of cancer, wherein the amphiphilic ligand conjugate includes a lipid, a peptide (e.g., a tumor associated antigen), and optionally a linker.

In some embodiments, the disclosure provides a kit including a container including a composition including an amphiphilic ligand conjugate, a TCR modified immune cell (e.g., a TCR modified T cell), an optional pharmaceutically acceptable carrier, and a package insert including instructions for administration of composition vaccine for expanding an immune cell expressing a T cell receptor in a subject, wherein the amphiphilic ligand conjugate includes a lipid, a peptide (e.g., a tumor associated antigen), and optionally a linker. In some embodiments, the kit further includes an adjuvant and instructions for administration of the adjuvant for expanding a TCR modified immune cell (e.g., a TCR modified T cell).

### EXAMPLES

Below are examples of specific embodiments for making the constructs and carrying out the methods described herein. These examples are provided for illustrative purposes only, and are not intended to limit the scope of the present invention in any way.

### Example 1. Vaccine Treatment for Boosting of TCR T Cells In Vivo

6-8 week old C57BL/6 mice were inoculated subcutaneously with 5×10⁵ B116F110 melanoma tumor cells on day −7. On day −1, tumors were measured with calipers and mice with equal tumor burden were selected for treatment. Mice were then randomized into different treatment cohorts. On day −1, mice were treated subcutaneously via tail base with either PBS, soluble vaccine including 10 μg of soluble gp100 peptide, or amphiphile vaccine including 10 μg of amphiphilic gp100 peptide and 1 nmol amphiphilic CpG. This was followed by tail vein intravenous injection of 1×10⁶ or 5×10⁶ T cells freshly isolated from splenocytes of 6-8 week old pmel-1 mice [B6.Cg-Thy1^{a}/CyTg(TcraTcrb)8Rest/J] via a negative T cell isolation kit on day 0. Subsequent booster doses of vaccine were given two times a week for two weeks via subcutaneous tail base injection on days 3, 7, 10, and 14.

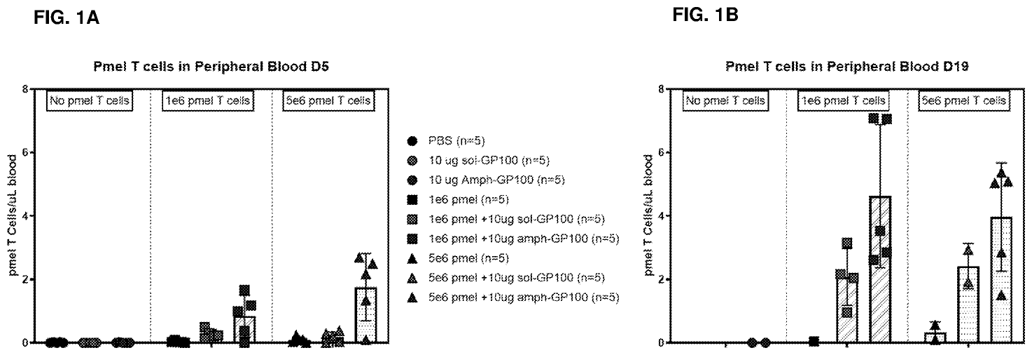

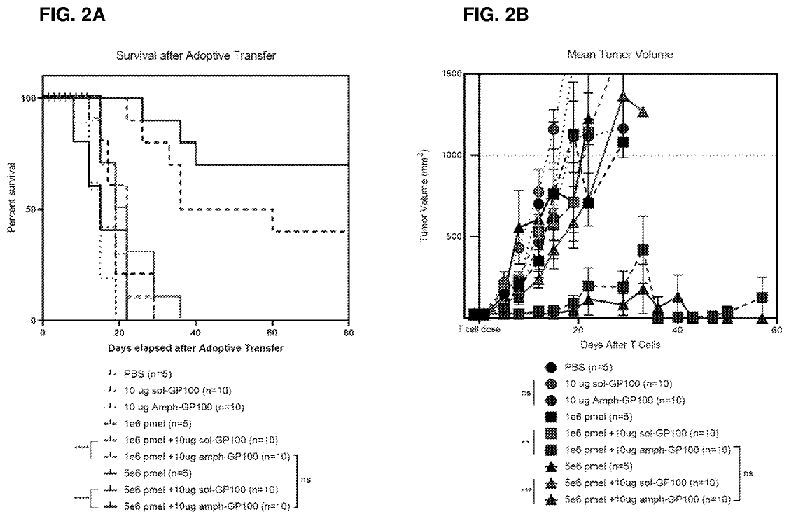

On days 5 and 19 post T cell infusion, whole blood was collected from mice via retro-orbital bleed. Red blood cells were lysed and T cells were washed with PBS and used for subsequent flow cytometry analysis. Adoptively transferred pmel T cells were identified by staining with murine anti-CD3, anti-CD4, anti-CD8, and anti-Thy1.1 antibodies by flow cytometry on a Cytoflex S Flow Cytometer (Beckman Coulter) for pmel T cells collected on day 5 (FIG. 1A) and day 19 (FIG. 1B). Overall survival and tumor volume were recorded over time (FIGS. 2A and 2B respectively). Mice were euthanized when tumor growth led to a volume greater than 1,000 mm³ by V=(0.5ab² where a is the longest length and b is the shortest length. The investigator was blinded when assessing the outcome. These results show that the amphiphilic vaccine expands tumor specific TCR T cells in vivo and that boosting with amphiphilic vaccine potently enhances TCR-T therapy to eliminate established solid tumors.

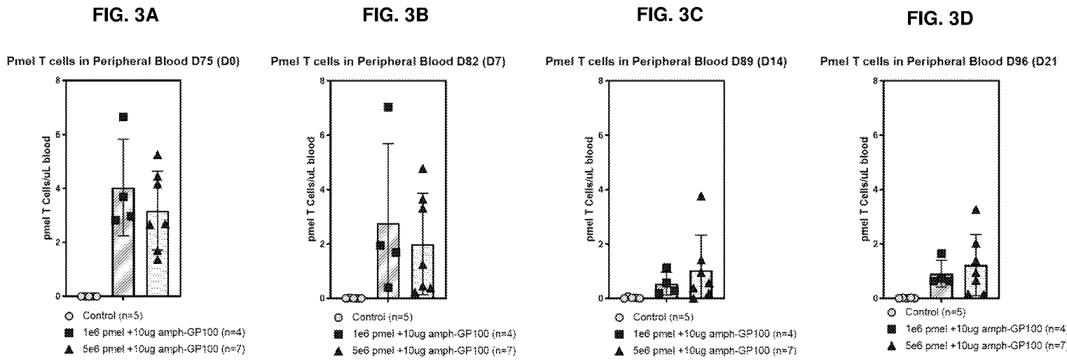

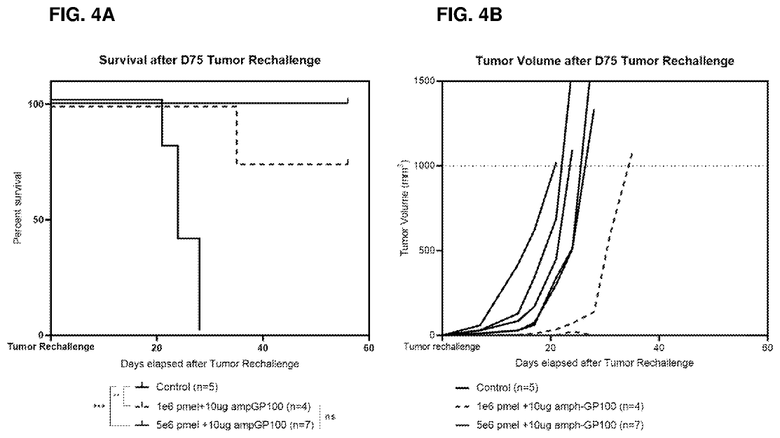

In another experiment, mice which had previously rejected a tumor following adoptive T cell transfer and amphiphile vaccine and a tumor naïve mouse control group were challenged with a second 5×10⁵ dose of B116F110 melanoma tumor cells on day 75 post initial adoptive transfer. No subsequent adoptive transfer or vaccination was performed. Peripheral blood was collected weekly, on days 75, 82, 89, and 96, following tumor implantation to analyze for T cells present in circulating blood. On days 0, 7, 14, and 21 post secondary tumor challenge, whole blood was collected from mice via retro-orbital bleed. Red blood cells were lysed and T cells were washed with PBS and used for subsequent flow cytometry analysis (FIGS. 3A-3D). Adoptively transferred pmel T cells were identified by staining with murine anti-CD3, anti-CD4, anti-CD8, and anti-Thy1.1 antibodies by flow cytometry on a Cytoflex S Flow Cytometer (Beckman Coulter). Overall mouse survival and tumor volume were recorded (FIGS. 4A and 4B respectively). Mice were euthanized when tumor growth led to a volume greater than 1,000 mm³ by V=(0.5ab² where a is the longest length and b is the shortest length. The investigator was blinded when assessing the outcome. These results show that boosting with an amphiphilic vaccine induces durable TCT-T cell responses and protection that lasts after a secondary tumor challenge.

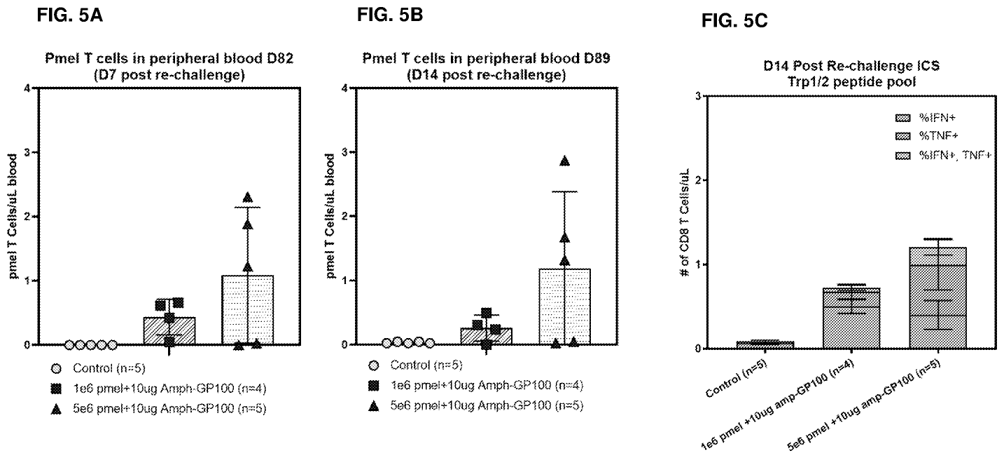

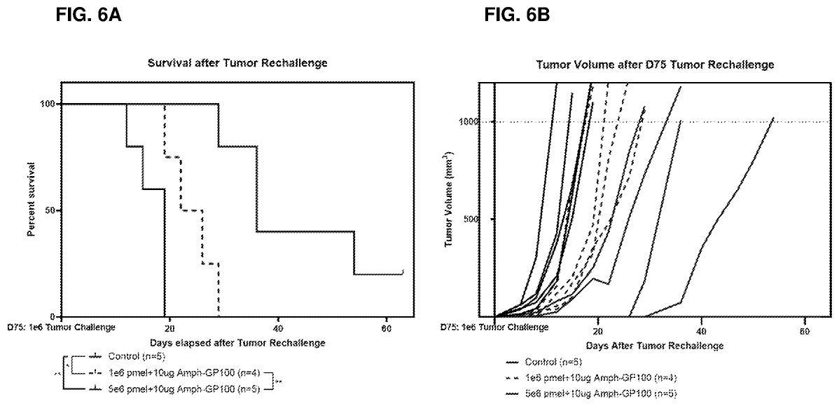

On days 7 and 14 post secondary tumor challenge, whole blood was collected from mice via retro-orbital bleed. Red blood cells were lysed and T cells were washed with PBS and used for subsequent flow cytometry analysis (FIGS. 5A and 5B). Adoptively transferred pmel T cells were identified by staining with murine anti-CD3, anti-CD4, anti-CD8, and anti-Thy1.1 antibodies by flow cytometry on a Cytoflex S Flow Cytometer (Beckman Coulter). Intracellular Cytokine Staining (ICS) was performed on day 14 post secondary tumor challenge by plating cells in a 96-well round bottom tissue culture plate and pulsing with a pool of Trp1 and Trp2 peptides at 5 μg of peptide per well overnight for 18 hours. Cells were then stained with murine anti-CD3, anti-CD4, anti-CD8, and anti-Thy1.1 antibodies and fixed according to manufacturer's instructions. Following this, the cells were washed multiple times, permeabilized, and stained with murine anti-Interferon gamma and anti-TNF alpha antibodies to analyze peptide specific cytokine secretion of peripheral blood T cells, see FIG. 5C. Tumor overall survival and tumor volume were recorded (FIGS. 6A and 6B respectively). These results show that boosting with amphiphilic vaccine induces durable TCR-T cell responses and protection lasting after a secondary challenge with a high tumor burden.

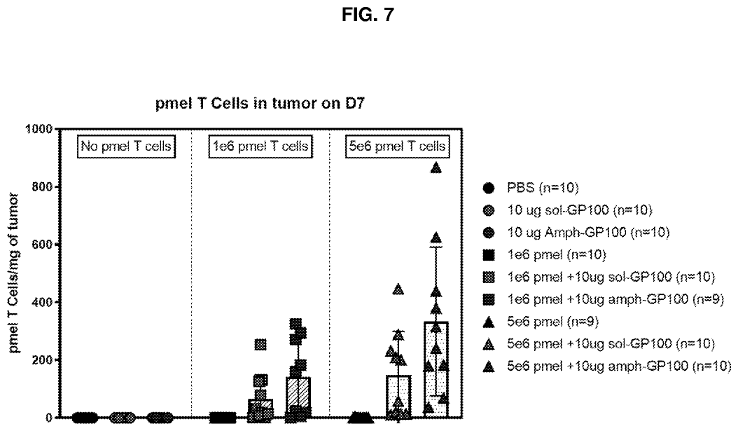

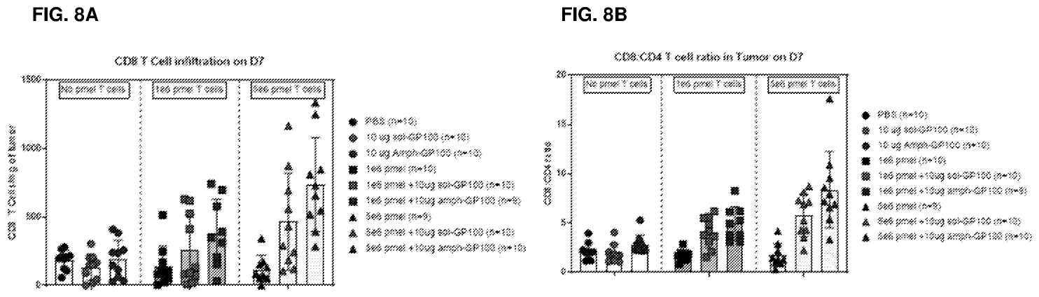

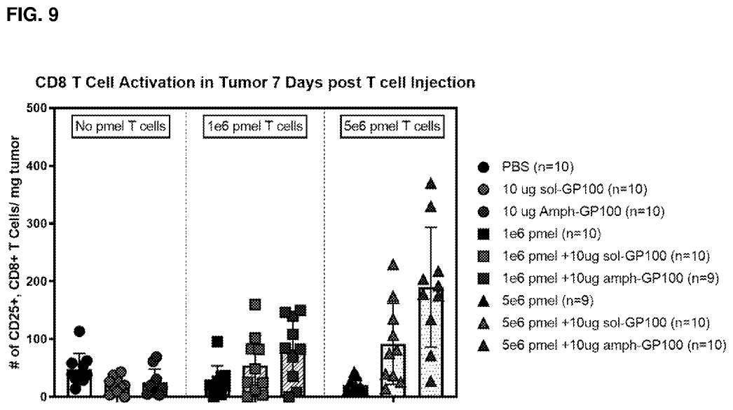

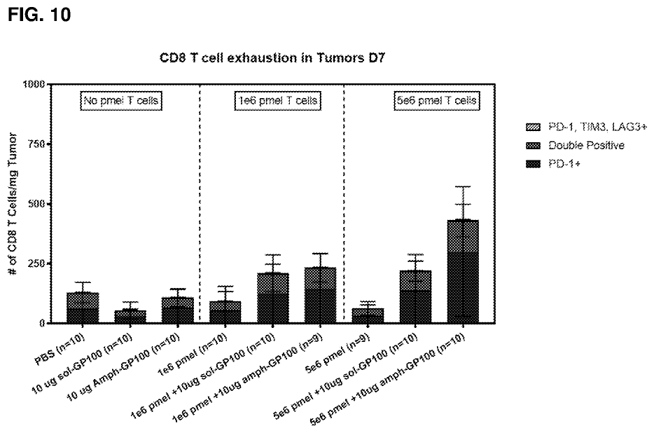

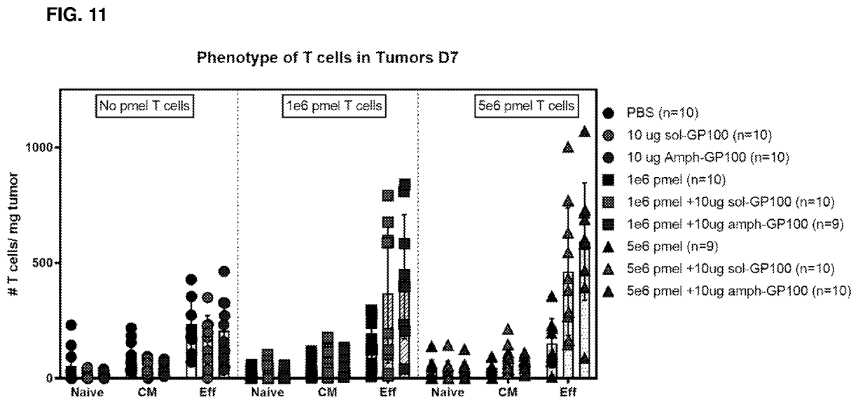

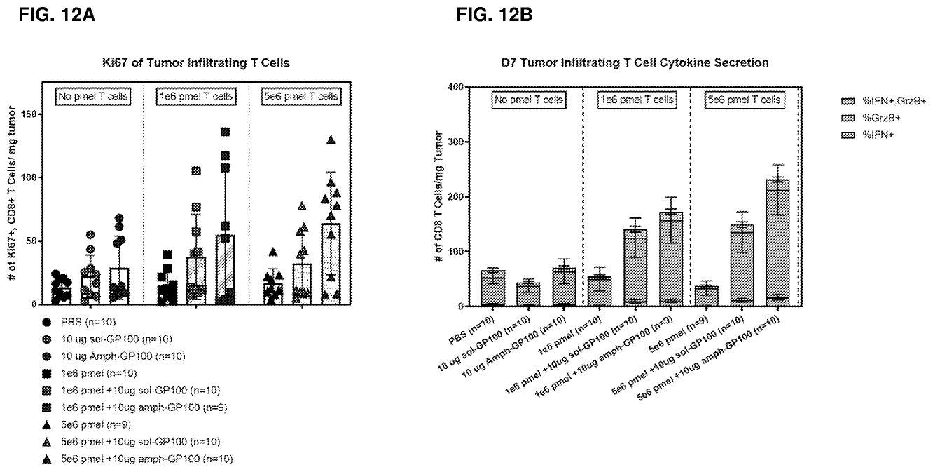

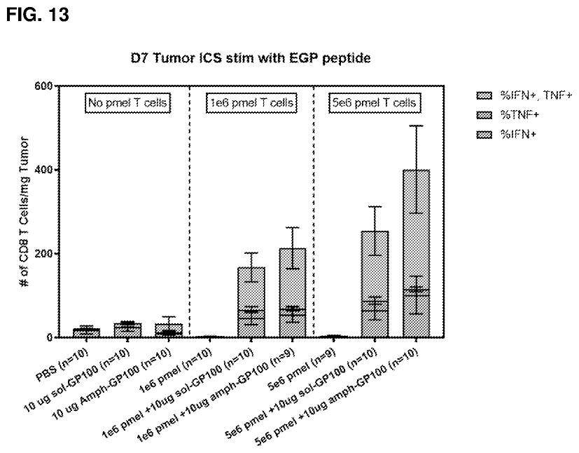

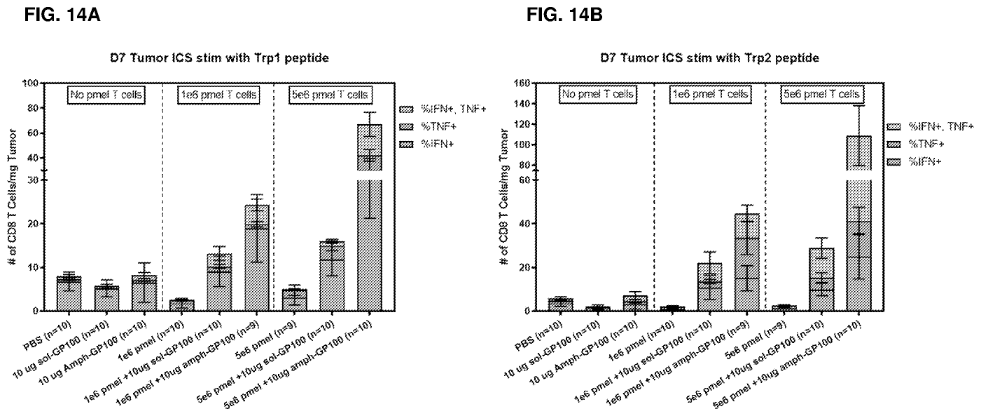

In another experiment, 6-8 week old C57BL/6 mice were inoculated subcutaneously with 5×10⁵ B116F110 melanoma tumor cells on day −7. On day −1, tumors were measured with calipers and mice with equal tumor burden were selected for treatment. Mice were then randomized into different treatment cohorts. On day −1, mice were treated subcutaneously via tail base with PBS, soluble vaccine including 10 μg of soluble gp100 peptide, or amphiphile vaccine including 10 μg of amphiphilic gp100 peptide and 1 nmol amphiphilic CpG. This was followed by tail vein intravenous injection of 1×10⁶ or 5×10⁶ T cells freshly isolated from splenocytes of 6-8 week old pmel-1 mice [B6.Cg-Thy1^{a}/CyTg(TcraTcrb)8Rest/J] via a negative T cell isolation kit (StemCell) on day 0. A subsequent booster dose of vaccine was given on day 3 via subcutaneous tail base injection. Mice were euthanized on day 7 post adoptive T cell transfer and tumors, peripheral blood, and inguinal lymph nodes were harvested for analysis. On day 7 post adoptive transfer, tumor samples were excised, weighed, mechanically dissociated, and passed through a 70 μm cell strainer. The homogenate was spun down at 1,500 RPM for 5 minutes at 4° C. to pellet the cells. Total tumor cells were counted and an aliquot was stained for flow cytometry analysis, see FIG. 7. After cells were counted and aliquots were processed for intracellular flow cytometry analysis, cells were pelleted and resuspended in 10% FBS/RPMI with Golgi plug for 6 hours at 37° C. Murine anti-CD3, anti-CD4, anti-CD8, anti-Thy1.1, anti-PD-1, anti-TIM-3, and anti-LAG-3 antibodies were used to identify the activation and exhaustion status of CD8+ T cells within the tumor sample by flow cytometry on a Cytoflex S Flow Cytometer (Beckman Coulter), see FIGS. 8A, 8B, 9 and 10. These results indicate that amphiphilic vaccination expands tumor specific TCR T cells, including activating and expanding CD8+ cells, within the tumor. Using this method the number of naïve, central memory (CM), and effector T cells per mg of tumor was characterized (FIG. 11). These results show that the amphiphilic vaccine expands effector T cells within the tumor. Following this, the cells were washed multiple times, permeabilized, stained with either murine anti-Ki67 or murine anti-Interferon gamma and anti-TNF alpha antibodies, and run on a Cytoflex S Flow Cytometer (Beckman Coulter) to analyze Ki67 levels or cytokine secretion of T cells within the tumor samples (FIGS. 12A and 12B). These results show that the amphiphilic vaccine induces proliferation and cytokine secretion of CD8+ T cells within the tumor. ICS was also performed on day 7 post T cell transfer by plating isolated cells from tumor homogenate in a 96-well round bottom tissue culture plate and pulsing with EGP peptides at 5 μg of peptide per well overnight for 18 hours. Cells were then stained with murine anti-CD3, anti-CD4, anti-CD8, and anti-Thy1.1 antibodies and fixed according to manufacturer's instructions. Following this, the cells were washed multiple times, permeabilized, stained with murine anti-Interferon gamma and anti-TNF alpha antibodies, and run on a Cytoflex S Flow Cytometer (Beckman Coulter) to analyze peptide specific cytokine secretion of T cells within the tumor samples (FIG. 13), where the results show that amphiphilic vaccination promoted tumor antigen specific cytokine section of tumor resident T cells. Additionally, ICS was performed on day 7 post T cell transfer by plating isolated cells from tumor homogenate in a 96-well round bottom tissue culture plate and pulsing with either Trp1 or Trp2 peptides at 5 μg of peptide per well overnight for 18 hours. Cells were then stained with murine anti-CD3, anti-CD4, anti-CD8, and anti-Thy1.1 antibodies and fixed according to manufacturer's instructions. Following this, the cells were washed multiple times, permeabilized, stained with murine anti-Interferon gamma and anti-TNF alpha antibodies, and run on a Cytoflex S Flow Cytometer (Beckman Coulter) to analyze peptide specific cytokine secretion of T cells within the tumor samples (FIGS. 14A and 14B). These results indicate that the amphiphilic vaccine induced epitope spread among CD8+ T cells within the tumor.

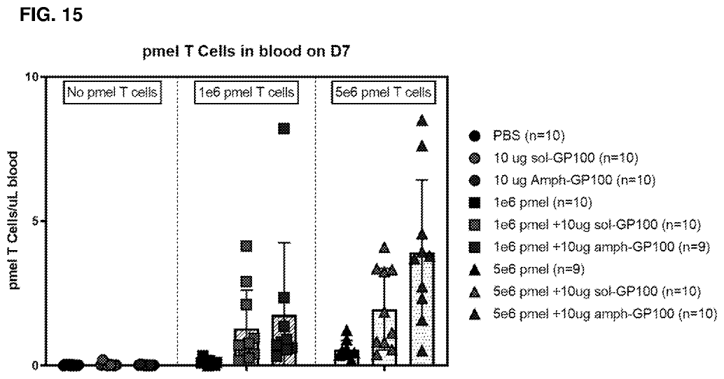

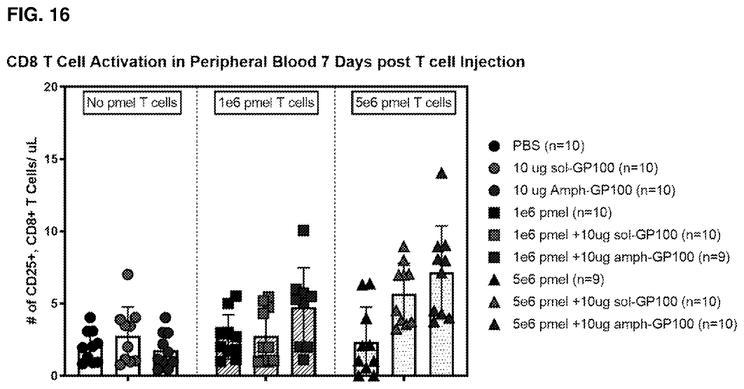

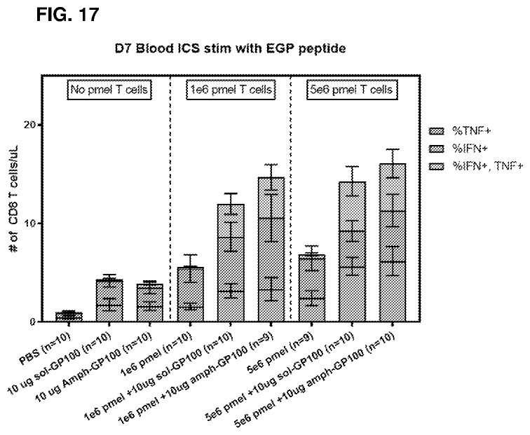

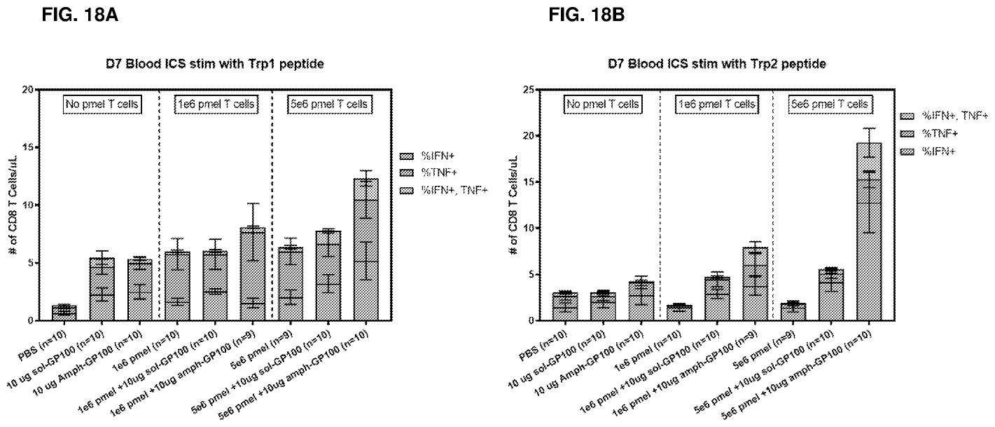

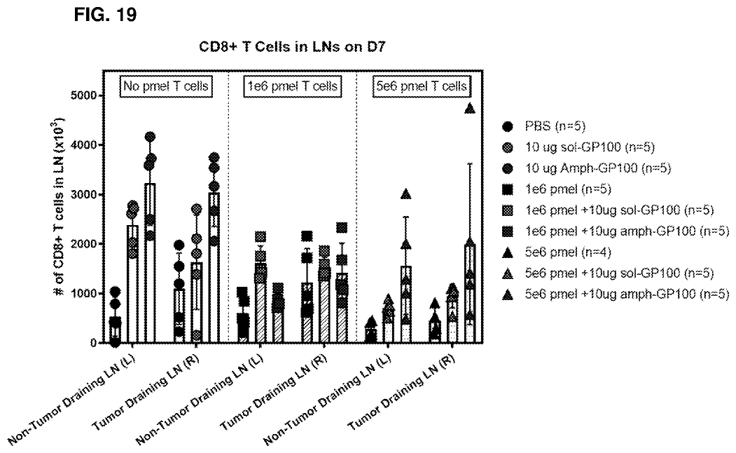

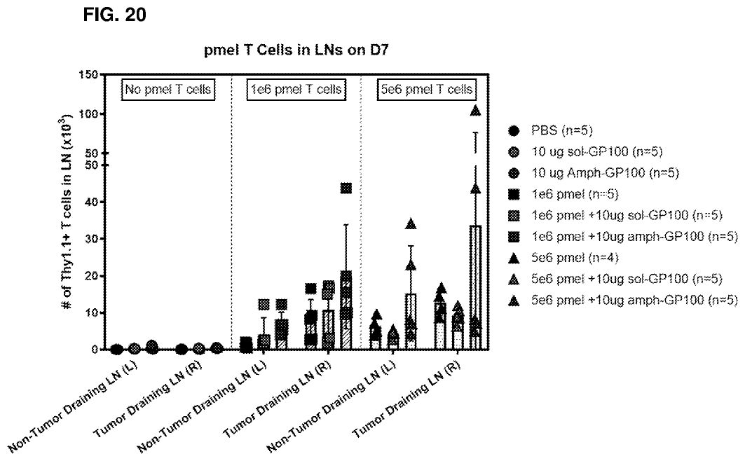

Also, on day 7 post adoptive transfer, peripheral blood was collected via retro-orbital bleed. Red blood cells were lysed and T cells were washed with PBS and used for subsequent flow cytometry analysis. Total cells were counted and an aliquot was stained for flow cytometry analysis (FIG. 15), where the results show that amphiphilic vaccination expands tumor specific TCR T cells in vivo in the peripheral blood. Murine anti-CD3, CD4, CD8 and anti-Thy1.1 antibodies were used to identify adoptively transferred pmel T cells in peripheral blood by flow cytometry on a Cytoflex S Flow Cytometer (Beckman Coulter) (FIG. 16). These results show that amphiphilic vaccination activates T cells in the peripheral blood. ICS was also performed on day 7 post T cell transfer by plating isolated cells from peripheral blood in a 96-well round bottom tissue culture plate and pulsing with EGP peptides at 5 μg of peptide per well overnight for 18 hours. Cells were then stained with murine anti-CD3, anti-CD4, anti-CD8, and anti-Thy1.1 antibodies and fixed according to manufacturer's instructions. Following this, the cells were washed multiple times, permeabilized, stained with murine anti-Interferon gamma and anti-TNF alpha antibodies, and run on a Cytoflex S Flow Cytometer (Beckman Coulter) to analyze peptide specific cytokine secretion of peripheral blood T cells (FIG. 17). The results indicate that amphiphilic vaccination induces tumor specific cytokine secretion of peripheral blood T cells. ICS was performed on day 7 post T cell transfer by plating isolated cells from peripheral blood in a 96-well round bottom tissue culture plate and pulsing with either Trp1 or Trp2 peptides at 5 μg of peptide per well overnight for 18 hours. Cells were then stained with murine anti-CD3, anti-CD4, anti-CD8, and anti-Thy1.1 antibodies and fixed according to manufacturer's instructions. Following this, the cells were washed multiple times, permeabilized, stained with murine anti-Interferon gamma and anti-TNF alpha antibodies, and run on a Cytoflex S Flow Cytometer (Beckman Coulter) to analyze peptide specific cytokine secretion of peripheral blood T cells (FIGS. 18A and 18B), which show that amphiphilic gp100 leads to epitope spread among T cells. On day 7 post adoptive transfer, inguinal lymph nodes (LN) were excised, weighed, mechanically dissociated, and passed through a 70 μm cell strainer. The homogenate was spun down at 1,500 RPM for 5 minutes at 4° C. to pellet the cells. Total cells were counted and an aliquot was stained for flow cytometry analysis (FIG. 20). Murine anti-CD3, anti-CD4, anti-CD8 and anti-Thy1.1 antibodies were used to identify the CD8+ T cells within the tumor sample by flow cytometry on a Cytoflex S Flow Cytometer (Beckman Coulter) (FIG. 19). These results indicate that the amphiphilic vaccine expands tumor specific TCR T cells, including CD*+ T cells, in vivo within the lymph nodes.

### Example 2. Stimulation of T Cells In Vitro

DC2.4, an immortalized dendritic cell line, was labeled with nothing, soluble gp100 peptides, or amphiphilic gp100 peptides overnight for 18 hours. The next day, naïve pmel T cells were isolated from splenocytes of 6-8 week old pmel-1 mice [B6.Cg-Thy1a/CyTg(TcraTcrb)8Rest/J] via a negative T cell isolation kit (StemCell). DC2.4 cells were washed 3 times and cultured with the naïve pmel T cells at a 1:1 ratio overnight for 24 hours.

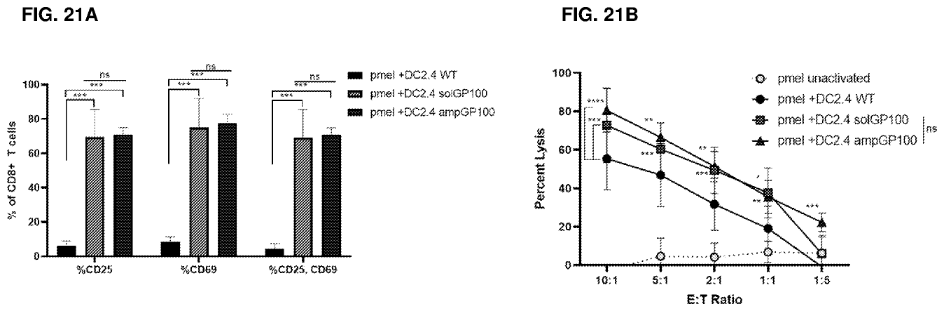

On day 1 after the co-culture was started, pmel T cells were counted and characterized by flow cytometry on a Cytoflex S flow cytometer (Beckman Coulter) after staining with murine anti-CD3, anti-CD4, anti-CD8, anti-Thy1.1, anti-CD25, and anti-CD69 antibodies (FIG. 21A). Additionally, the cytolytic capacity of the pmel T cells post co-culture was assessed through a 24 hour luciferase killing assay. 5×10⁴ B16F10 tumor cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, One-Glo reagent (Promega) was added to each well. Emitted luminescence of each sample (sample signal) was detected by a Synergy H1 Hybrid plate reader (BioTek). Percent lysis was determined as [1−(sample signal/max signal)]×100, see FIG. 21B.

In another experiment, naïve pmel T cells were isolated from splenocytes of 6-8 week old pmel-1 mice [B6.Cg-Thy1^{a}/CyTg(TcraTcrb)8Rest/J]. Pmel-1 spleens were harvested and following tissue dissociation and red blood cell lysis, T cells were isolated by negative bead selection and subsequently activated with CD3/CD28 Dynabeads at a bead:cell ratio of 1:2 (Invitrogen). Cells were expanded in vitro by culturing in RPMI1640 supplemented with 10% heat-inactivated FBS, sodium pyruvate, 1% penicillin/streptomycin, 2-mercaptoethanol, and 100 IU/mL of recombinant human IL2. 24 and 48 hours after initial expansion, T cells were spinoculated with viral supernatant collected from mCherry transfected Phoenix-ECO cells. Murine T cells were transduced by centrifugation on RetroNectin coated plates with retroviral supernatant from viral packaging cells. Transduction efficiency was confirmed by expression of mCherry by flow cytometry on a Cytoflex S flow cytometer (Beckman Coulter).

Transduced T cells were rested for 72 hours. On day 0, before the co-culture was started, pmel T cells were counted and characterized by flow cytometry on a Cytoflex S flow cytometer (Beckman Coulter) after staining with murine anti-CD3, anti-CD4, anti-CD8, anti-CD25, and anti-CD69 antibodies. DC2.4, an immortalized dendritic cell line, was labeled with nothing, soluble, or amp GP100 peptides overnight for 18 hours. DC2.4 cells were washed 3 times and cultured with the transduced and rested pmel T cells at a 1:1 ratio overnight for 24 hours.

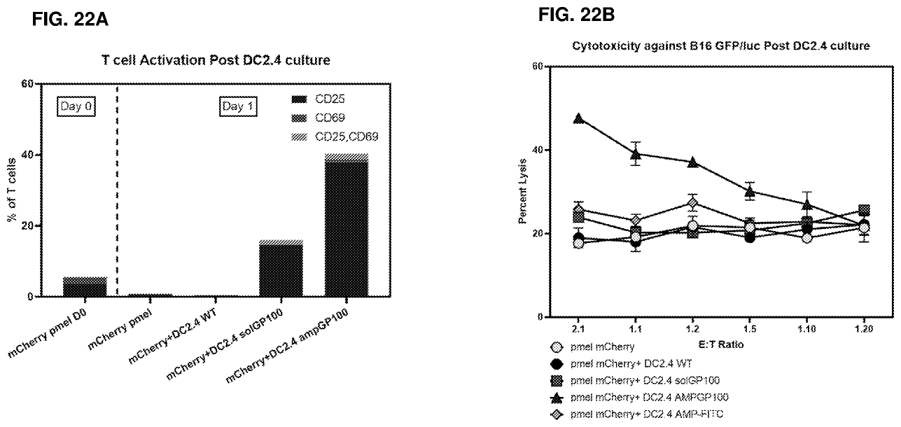

On Day 1 after the co-culture was started, pmel T cells were counted and characterized by flow cytometry on a Cytoflex S flow cytometer (Beckman Coulter) after staining with murine anti-CD3, anti-CD4, anti-CD8, anti-Thy1.1, anti-CD25, and anti-CD69 antibodies (FIG. 22A). Additionally, the cytolytic capacity of the pmel T cells post co-culture was assessed through a 24 hour luciferase killing assay. 5×10⁴ B16F10 tumor cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, One-Glo reagent (Promega) was added to each well. Emitted luminescence of each sample (sample signal) was detected by a Synergy H1 Hybrid plate reader (BioTek). Percent lysis was determined as [1−(sample signal/max signal)]×100, see FIG. 22B. These results show that the naïve pmel T cells were activated in vitro with the amphiphilic gp100 labeled DC2.4s.

### Example 3. Activation of Dendritic Cells in Lymph Nodes

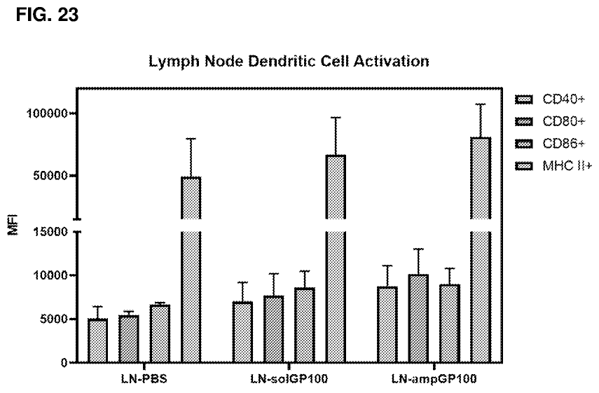

6-8 week old C57BL/6 mice were vaccinated subcutaneously via tail base injection with either PBS, soluble vaccine including 10 μg of soluble gp100 peptide, or amphiphile vaccine including 10 μg of amphiphilic gp100 peptide and 1 nmol amphiphilic CpG. After 48 hours, the mice were euthanized and inguinal lymph nodes were extracted and mechanically dissociated and filtered through a 70 μm cell strainer to form a lymph node homogenate. The homogenate was spun down at 1,500 RPM for 5 minutes at 4° C. to pellet the cells. Total cells were counted and an aliquot was stained for flow cytometry analysis. Murine anti-CD3, anti-CD19, anti-F4/80, anti-CD11b, anti-CD11c, anti-MHC Class II, anti-CD40, anti-CD80, and anti-CD86 antibodies were used to analyze the activation of murine dendritic cells in the lymph nodes of vaccinated mice by flow cytometry on a Cytoflex S Flow Cytometer (Beckman Coulter), see FIG. 23. These results indicate that boosting with the amphiphilic vaccine enhances the activation of the dendritic cells in the lymph nodes.

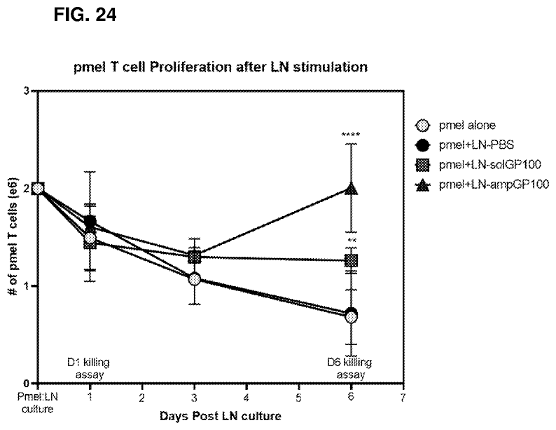

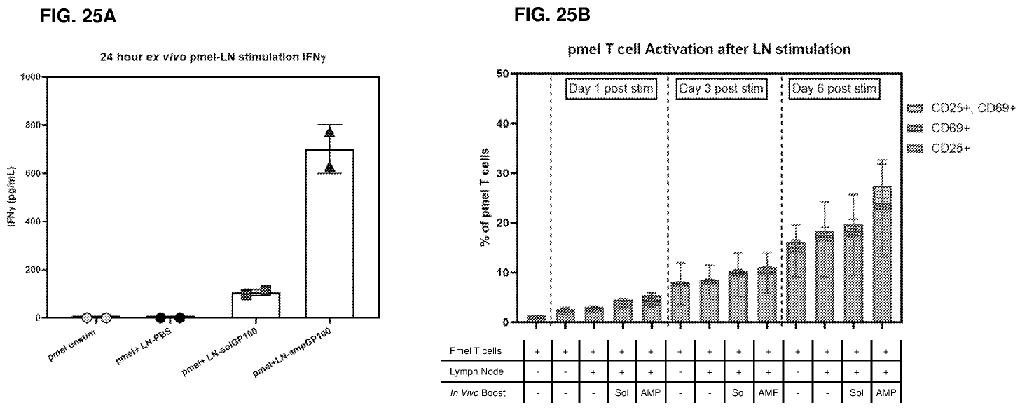

Simultaneously, naïve pmel T cells were isolated from splenocytes of 6-8 week old pmel-1 mice [B6.Cg-Thy1^{a}/CyTg(TcraTcrb)8Rest/J] via a negative T cell isolation kit (StemCell). The lymph node homogenate was cultured with the naïve pmel T cells at a 1:1 ratio overnight for 24 hours to activate the T cells. After 24 hour co-culture, the supernatant fluid was collected and analyzed for T cell cytokine production using a Th17 murine cytokine kit on a Luminex LX200 analyzer (Millipore) (FIG. 25A). On Days 1, 3, and 6 after the co-culture was started, pmel T cells were counted for proliferation analysis (FIG. 24) and characterized by flow cytometry on a Cytoflex S flow cytometer (Beckman Coulter) after staining with murine anti-CD3, anti-CD4, anti-CD8, anti-Thy1.1, anti-CD25, and anti-CD69 antibodies, as shown in FIG. 25B. These results indicate that boosting with the amphiphilic vaccine in the lymph nodes enhances proliferation and activation of TCR T cells.

In another experiment, naïve pmel T cells were isolated from splenocytes of 6-8 week old pmel-1 mice [B6.Cg-Thy1^{a}/CyTg(TcraTcrb)8Rest/J] via a negative T cell isolation kit (StemCell). The lymph node homogenate was cultured with the naïve pmel T cells at a 1:1 ratio overnight for 24 hours to activate the T cells.

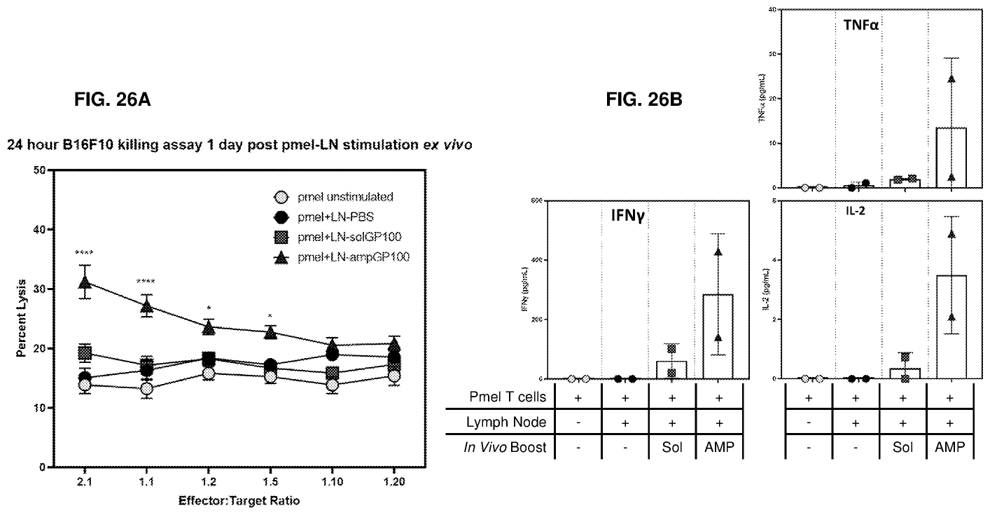

On day 1 after the co-culture was started, pmel T cells were counted and characterized by flow cytometry on a Cytoflex S flow cytometer (Beckman Coulter) after staining with murine anti-CD3, anti-CD4, anti-CD8, anti-Thy1.1, anti-CD25, and anti-CD69 antibodies. Additionally, the cytolytic capacity of the pmel T cells post co-culture was assessed through a 24 hour luciferase killing assay (FIG. 26A). 5×10⁴ B16F10 tumor cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, the supernatant fluid was collected and analyzed for T cell cytokine production using a Th17 murine cytokine kit on a Luminex LX200 analyzer (Millipore) and then One-Glo reagent (Promega) was added to each well, see FIG. 26B. Emitted luminescence of each sample (sample signal) was detected by a Synergy H1 Hybrid plate reader (BioTek). Percent lysis was determined as [1−(sample signal/max signal)]×100.

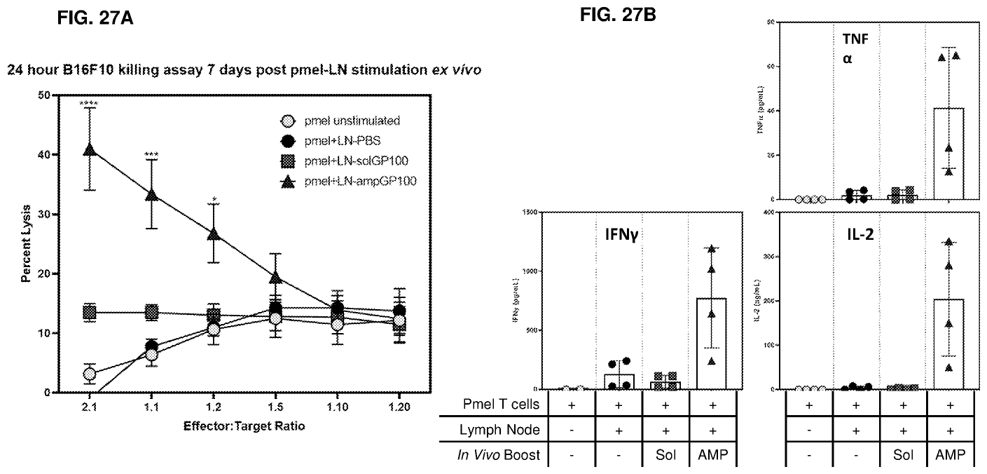

On day 6 after the co-culture was started, pmel T cells were counted and characterized by flow cytometry on a Cytoflex S flow cytometer (Beckman Coulter) after staining with murine anti-CD3, anti-CD4, anti-CD8, anti-Thy1.1, anti-CD25, and anti-CD69 antibodies. Additionally, the cytolytic capacity of the pmel T cells post co-culture was assessed through a 24 hour luciferase killing assay (FIG. 27A). 5×10⁴ B16F10 tumor cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, the supernatant fluid was collected and analyzed for T cell cytokine production using a Th17 murine cytokine kit on a Luminex LX200 analyzer (Millipore) and then One-Glo reagent (Promega) was added to each well, see FIG. 27B. Emitted luminescence of each sample (sample signal) was detected by a Synergy H1 Hybrid plate reader (BioTek). Percent lysis was determined as [1−(sample signal/max signal)]×100. These results indicate that boosting with amphiphilic vaccine in the lymph nodes enhances functionality of TCR-T cells.

### Example 4. Treatment of Large, Established Tumors with Amphiphile Boosting

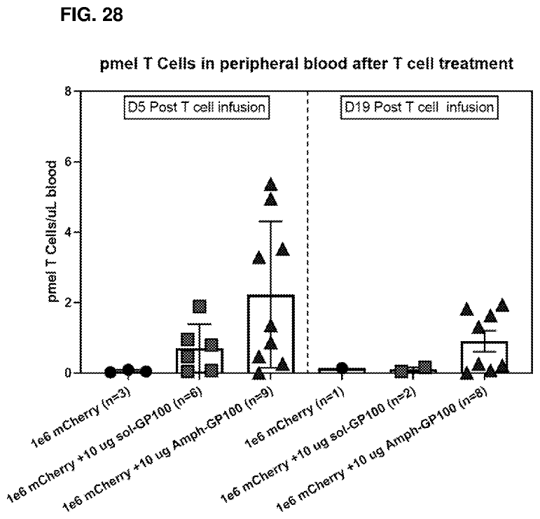

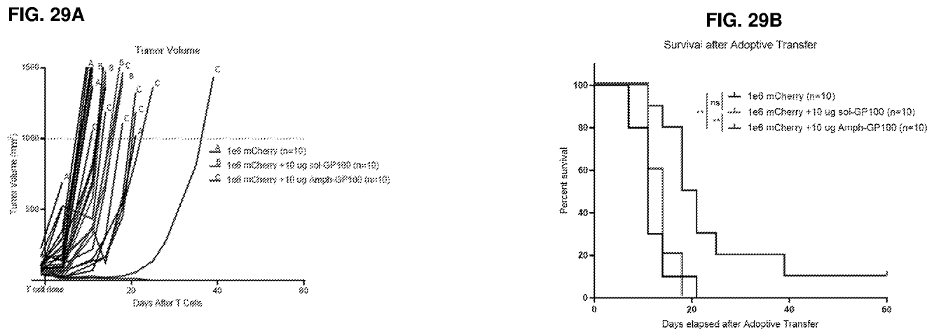

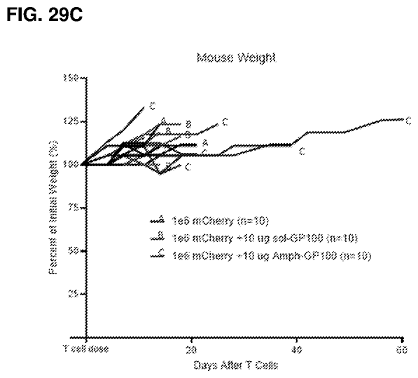

6-8 week old C57BL/6 mice were inoculated subcutaneously with 5×10⁵ B16F10 melanoma tumor cells on day −10 to generate larger, established tumors. On day −1, tumors were measured with calipers and mice with equal tumor burden were selected for treatment. Mice were then randomized into different treatment cohorts. On day −1, mice were treated subcutaneously via tail base with PBS, soluble vaccine including 10 μg of soluble gp100 peptide, or amphiphile vaccine including 10 μg of amphiphilic gp100 peptide and 1 nmol amphiphilic CpG. This was followed by tail vein intravenous injection of 1×10⁶ mCherry transduced pmel T cells. A subsequent booster dose of vaccine was given on day 3 via subcutaneous tail base injection. Tumor volume, overall survival, and mouse weight were recorded as shown in FIGS. 29A, 29B, and 29C respectively. These results show that boosting with amphiphilic vaccine I combination with preconditioning potently enhances TCR-T therapy to delay growth of large established solid tumors. Mice were euthanized when tumor growth led to a volume greater than 1,000 mm³ by V=(0.5ab² where a is the longest length and b is the shortest length. The investigator was blinded when assessing the outcome. On days 5 and 19 post T cell infusion, whole blood was collected from mice via retro-orbital bleed. Red blood cells were lysed and T cells were washed with PBS and used for subsequent flow cytometry analysis. Adoptively transferred pmel T cells were identified by staining with murine anti-CD3, anti-CD4, anti-CD8, and anti-Thy1.1 antibodies and running on a Cytoflex S Flow Cytometer (Beckman Coulter) (FIG. 28). These results show that amphiphilic vaccination expands tumor specific TCR T cells in large, established tumor bearing hosts.

pmel-1 mice [B6.Cg-Thy1^{a}/CyTg(TcraTcrb)8Rest/J] were euthanized and spleens were harvested. Following tissue dissociation and red blood cell lysis, T cells were isolated by negative bead selection and subsequently activated with CD3/CD28 Dynabeads at a bead:cell ratio of 1:2 (Invitrogen). Cells were expanded in vitro by culturing in RPMI1640 supplemented with 10% heat-inactivated FBS, sodium pyruvate, 1% penicillin/streptomycin, 2-mercaptoethanol, and 100 IU/mL of recombinant human IL2. 24 and 48 hours after initial expansion, T cells were spinoculated with viral supernatant collected from transfected Phoenix-ECO cells. Murine T cells were transduced by centrifugation on RetroNectin coated plates with retroviral supernatant from viral packaging cells.

### Example 5. Treatment of Large, Established Tumors with Amphiphile Boosting and Preconditioning

6-8 week old C57BL/6 mice were inoculated subcutaneously with 5×10⁵ B16F10 melanoma tumor cells on day −10. After 8 days of tumor growth, on day −2, mice were subjected to 5 Gy whole body gamma irradiation. On day −1, tumors were measured with calipers and mice with equal tumor burden were selected for treatment. Mice were then randomized into different treatment cohorts. On day −1, mice were treated subcutaneously via tail base with PBS, soluble vaccine including 10 μg of soluble gp100 peptide, or amphiphile vaccine including 10 μg of amphiphilic gp100 peptide and 1 nmol amphiphilic CpG. This was followed by tail vein intravenous injection of 1×10⁵ or 1×10⁶ T cells freshly isolated from splenocytes of 6-8 week old pmel-1 mice [B6.Cg-Thy1^{a}/CyTg(TcraTcrb)8Rest/J] via a negative T cell isolation kit on day 0. A subsequent booster dose of either soluble of amphiphilic vaccine was given on day 3, via subcutaneous tail base injection. Tumor volume, mouse weight, and overall survival were recorded. Mice were euthanized when tumor growth led to a volume greater than 1,000 mm³ by V=(0.5ab² where a is the longest length and b is the shortest length. The investigator was blinded when assessing the outcome.

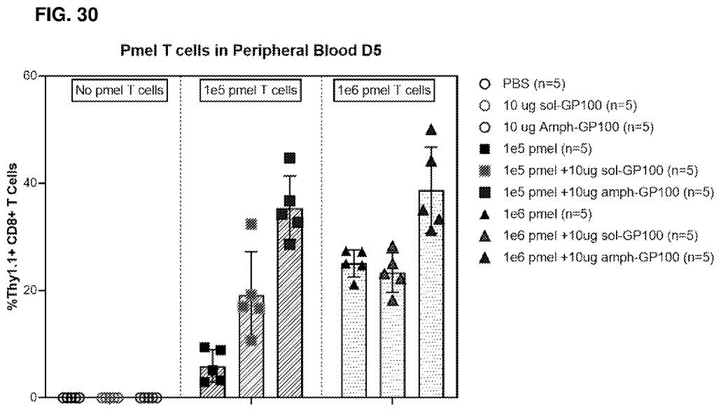

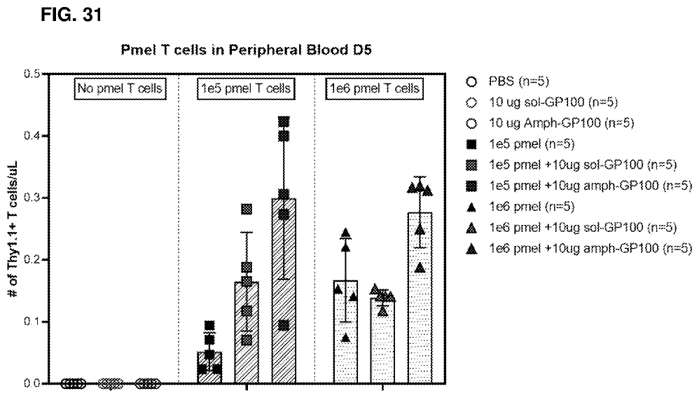

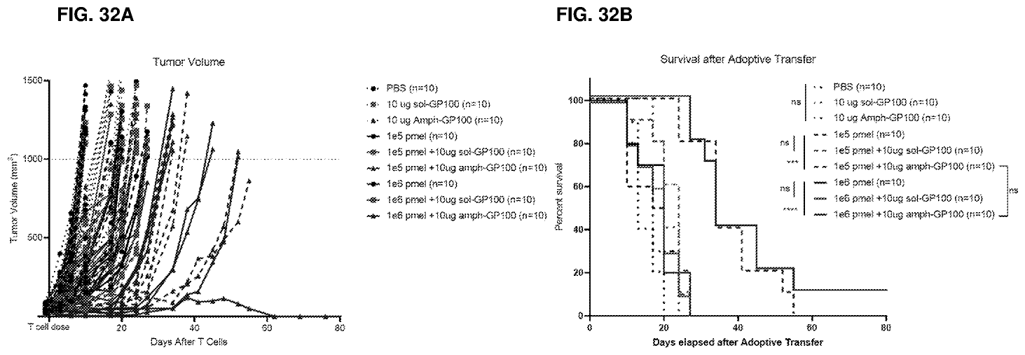

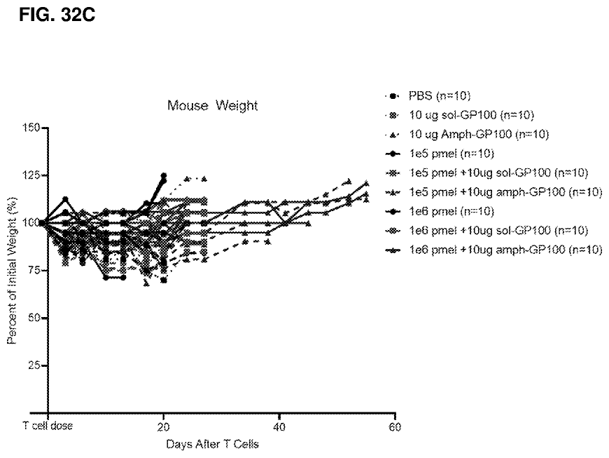

On day 5 post T cell infusion, whole blood was collected from mice via retro-orbital bleed. Red blood cells were lysed and T cells were washed with PBS and used for subsequent flow cytometry analysis. Adoptively transferred pmel T cells in peripheral blood were identified by staining with murine anti-CD3, anti-CD4, anti-CD8, and anti-Thy1.1 antibodies and running on a Cytoflex S Flow Cytometer (Beckman Coulter) (FIGS. 30 and 31). Tumor volume, overall survival, and mouse weight were recorded as shown in FIGS. 32A, 32B, and 32C respectively. Mice were euthanized when tumor growth led to a volume greater than 1,000 mm³ by V=(0.5ab² where a is the longest length and b is the shortest length. The investigator was blinded when assessing the outcome. These results show that amphiphilic vaccination expands tumor specific TCR T cells in lymphodepleted hosts and boosting with amphiphilic vaccine in combination with preconditioning potently enhances TCR-T therapy to delay the growth of large established solid tumors.

### Example 6. Amphiphilic Boosters Enhance TCR-T Therapy to Eliminate Established Solid Tumors

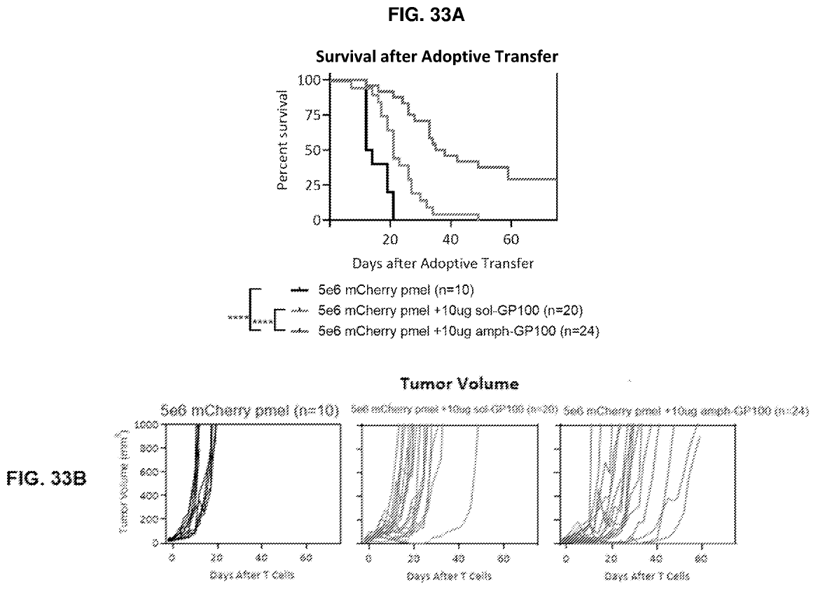

6-8 week old C57BL/6 mice were inoculated subcutaneously with 5×10⁵ B16F10 melanoma tumor cells on day −10. On day −1, tumors were measured with calipers and mice with equal tumor burden were selected for treatment. Mice were then randomized into different treatment cohorts. On day −1, mice were treated subcutaneously via tail base with PBS, 10 μg soluble gp100, or 10 μg amphiphilic gp100. This was followed by a day 0 tail vein intravenous injection of 5×10⁶ T cells previously isolated from splenocytes of 6-8 week old pmel-1 mice [B6.Cg-Thy1^{a}/CyTg(TcraTcrb)8Rest/J] via a negative T cell isolation kit and retrovirally transduced with mCherry. Subsequent booster doses of vaccine were given two times a week for two weeks via subcutaneous tail base injection on days 3, 7, 10, and 14. Tumor volume and overall survival were recorded (FIGS. 33A-33B). Mice were euthanized when tumor growth led to a volume greater than 1,000 mm³ by V=(0.5ab² where a is the longest length and b is the shortest length.

### Example 7. Effect of AMP-Boosting on Providing Protection Against Secondary Tumor Rechallenge

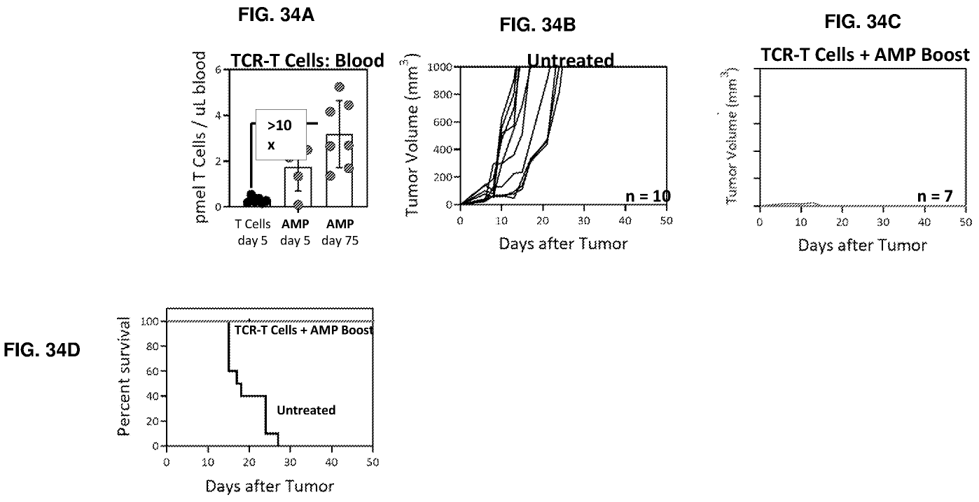

Mice which had previously rejected tumor following adoptive T cell transfer and the amphiphilic gp100 and a tumor naïve control group were challenged with a second 5×10⁵ dose of B116F110 melanoma tumor cells on day 75 post initial adoptive transfer. No subsequent adoptive transfer or vaccination was performed. Peripheral blood was collected following tumor implantation to analyze for T cells present in circulating blood by flow cytometry (FIG. 34A). Tumor volume and overall survival were recorded (FIGS. 34B-34D). Mice were euthanized when tumor growth led to a volume greater than 1,000 mm³ by V=(0.5ab² where a is the longest length and b is the shortest length. The investigator was blinded when assessing the outcome.

The amphiphile antigen vaccination led to enhanced persistence of tumor specific TCR T cells and protection against secondary tumor challenge.

### Example 8. AMP-Boosting Expands TCR-T Cells in Blood and Lymph Nodes and Enhances Tumor Infiltration

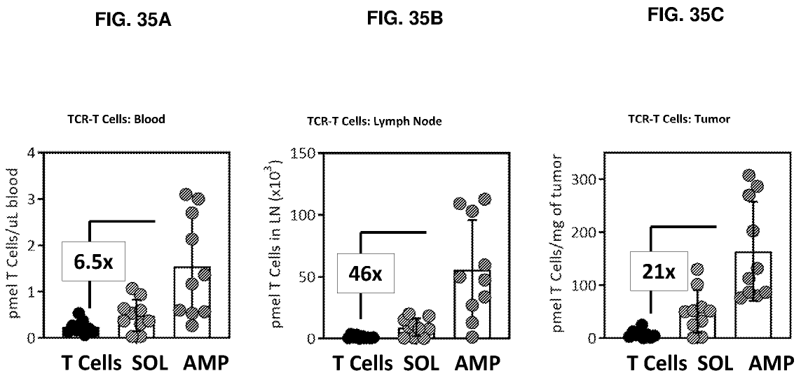

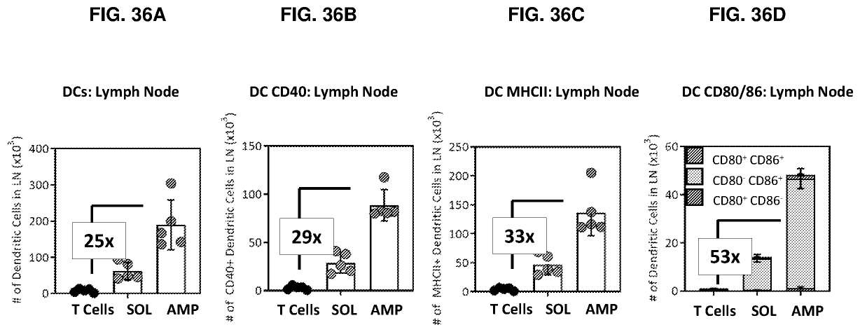

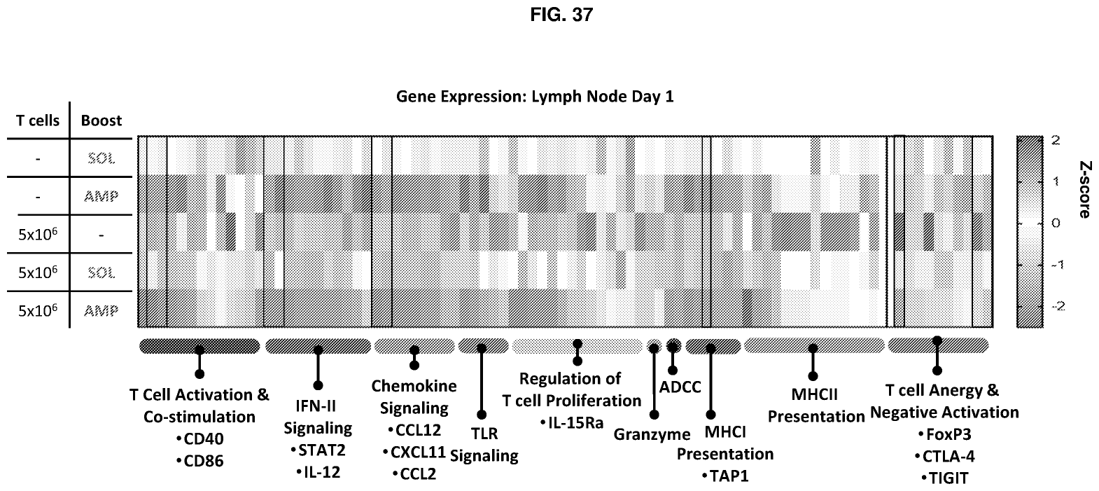

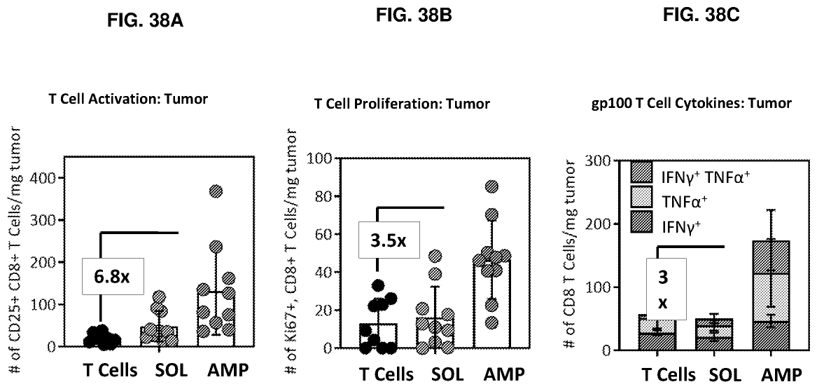

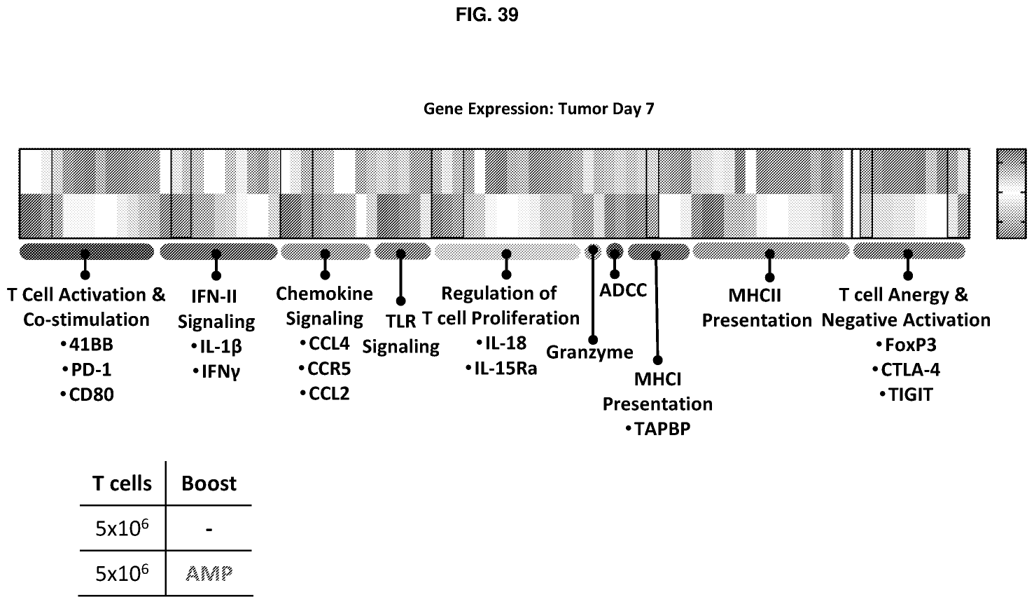

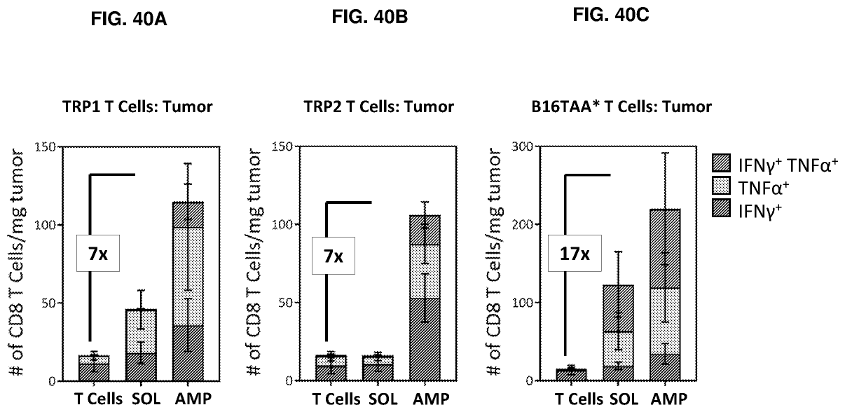

6-8 week old C57BL/6 mice were inoculated subcutaneously with 5×10⁵ B116F110 melanoma tumor cells on day −10. On day −1, tumors were measured with calipers and mice with equal tumor burden were selected for treatment. Mice were then randomized into different treatment cohorts. On day −1, mice were treated subcutaneously via tail base with PBS, 10 μg soluble gp100, or 10 μg amphiphilic gp100. This was followed by DO tail vein intravenous injection of 5×10⁶ T cells previously isolated from splenocytes of 6-8 week old pmel-1 mice [B6.Cg-Thy1^{a}/CyTg(TcraTcrb)8Rest/J] via a negative T cell isolation kit (StemCell) and retrovirally transduced with mCherry. A subsequent booster dose of vaccine was given on D3 via subcutaneous tail base injection. Peripheral blood was collected 5 days after T cell injection to analyze for T cells present in circulating blood (FIG. 35A). Mice were euthanized on D7 post adoptive T cell transfer and tumors and inguinal lymph nodes were harvested for analysis by flow cytometry for the amount of pmel T cells in the lymph node (FIG. 35B), the amount of pmel cells per mg of tumor (FIG. 35C), the amount of dendritic cells in the lymph nodes (FIG. 36A), the amount of CD40+ and MHCII+ dendritic cells (FIGS. 36B and 36C), and the number of dendritic cells with CD80 and/or CD86 (FIG. 36D). Harvested tumor cells were also analyzed through Intracellular Cytokine staining after overnight stimulation with tumor associated peptides as indicated (FIGS. 38A-38C and 40A-40C). In a separate experiment, mice were euthanized on D1 or D7 post adoptive T cell transfer and inguinal lymph nodes were harvested for RNA sequencing analysis by a 561 gene mouse immunology nanostring panel (Canopy Biosciences) (FIGS. 37 and 39).

### Example 9. Amphiphile Vs. Soluble Cognate Antigen Vaccination Ex Vivo

6-8 week old C57BL/6 mice were vaccinated subcutaneously via tail base injection with either PBS, 10 μg soluble gp100, or 10 μg amphiphilic gp100. After 48 hours, the mice were euthanized and inguinal lymph nodes were extracted and mechanically dissociated and filtered through a 70 μm cell strainer to form a lymph node homogenate. The homogenate was spun down at 1,500 RPM for 5 minutes at 4° C. to pellet the cells.

Previously, murine T cells were isolated from splenocytes of 6-8 week old pmel-1 mice [B6.Cg-Thy1^{a}/CyTg(TcraTcrb)8Rest/J] via a negative T cell isolation kit and retrovirally transduced with mCherry and rested prior to Lymph node culture.

The lymph node homogenate was cultured with the previously activated, transduced, and rested pmel T cells at a 1:1 ratio overnight for 24 hours to activate the T cells.

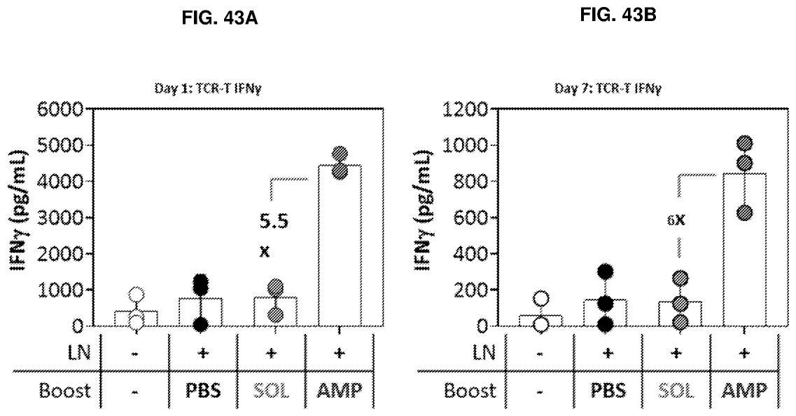

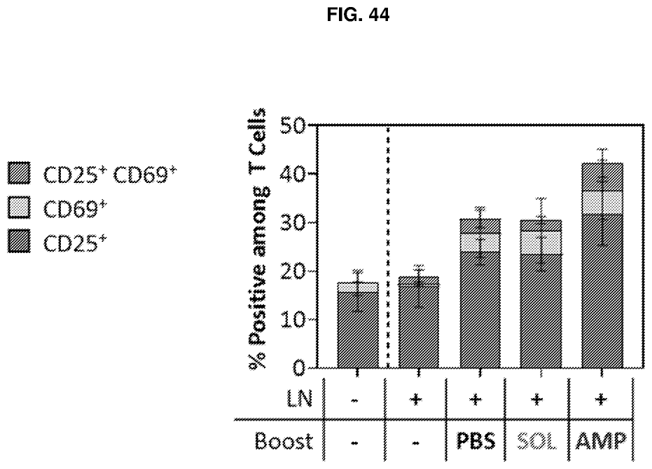

On Days 1, 3, and 6 after the co-culture was started, pmel T cells were counted for proliferation analysis and characterized by flow cytometry on a Cytoflex S flow cytometer (Beckman Coulter) after staining with murine anti-CD3, anti-CD4, anti-CD8, anti-Thy1.1, anti-CD25, and anti-CD69 antibodies (FIG. 44). On Days 1 and 6 after co-culture, Supernatant liquid was collected from TCR T Cell: Lymph Node cultures and analyzed by Luminex for secretion of INFg murine cytokines. These figures are generated from triplicate wells from 2 independent experiments (FIGS. 43A and 43B).

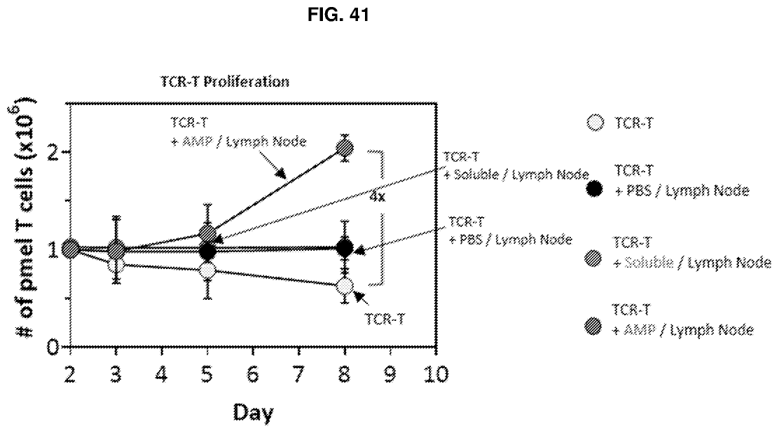

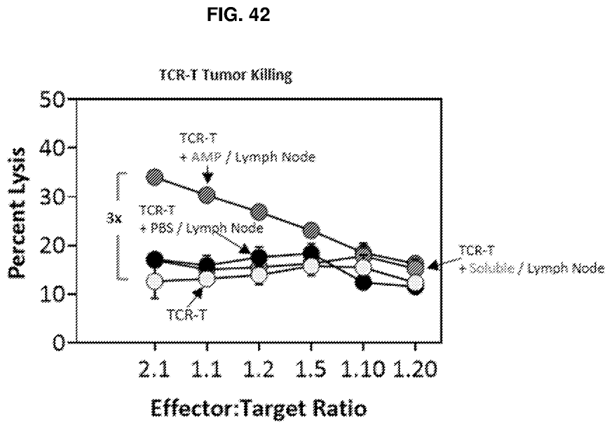

After overnight culture with lymph node homogenate, activated pmel T cells were counted (FIG. 41) and cultured at various Effector to Target Ratios with B16F10 target cells expressing luciferase gene to determine specific lysis (FIG. 42). 5×10⁴ target cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, One-Glo reagent (Promega) was added to each well. Emitted luminescence of each sample (sample signal) was detected by a Synergy H1 Hybrid plate reader (BioTek). Percent lysis was determined as [1−(sample signal/max signal)]×100. These figures are generated from triplicate wells from 2 independent experiments.

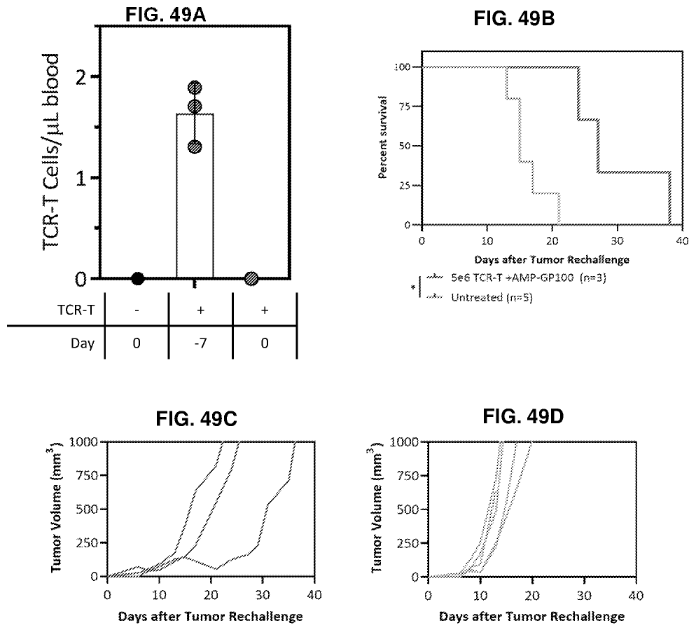

Mice which had previously rejected tumor following adoptive T cell transfer and 10 μg amphiphilic gp100 vaccine and a tumor naïve control group were challenged with a second 5×10⁵ dose of B16F10 melanoma tumor cells on day 75 post initial adoptive transfer. One week prior, the adoptively transferred cells were depleted by intraperitoneal injection of an anti-thy1.1 antibody. This antibody was continued weekly for the duration of the experiment. No subsequent adoptive transfer or vaccination was performed, indicating that the anti-tumor effect demonstrated was related to the endogenous antigen spread effect. Peripheral blood was collected prior to depletion and following depletion, which showed successful reduction of adoptively transferred T cells (FIG. 49A). Tumor volume and overall survival were recorded (FIGS. 49B, 49C, and 49D). Mice were euthanized when tumor growth led to a volume greater than 1,000 mm³ by V=(0.5ab² where a is the longest length and b is the shortest length. The investigator was blinded when assessing the outcome.

### Example 10. Amphiphile Boosting of KRAS Specific TCR T Cells

Human peripheral blood mononuclear cells were isolated from an HLA A*11:01 or HLA C*08:02 donor leukopack (StemExpress). Monocytes and T cells were further isolated by a negative monocyte or T cell isolation (StemCell) kit, respectively. Following negative bead selection, human T cells were subsequently activated with CD3/CD28 Dynabeads at a bead:cell ratio of 1:1 (Invitrogen). Cells were expanded in vitro by culturing in RPMI1640 supplemented with 10% heat-inactivated FBS, sodium pyruvate, 1% penicillin/streptomycin, 2-mercaptoethanol, and 50 IU/mL of recombinant human IL-2. 24 and 48 hours after initial expansion, T cells were spinoculated with viral supernatant collected from Phoenix-Ampho cells transfected with the TCR 701 KRAS G12D specific TCR T Cell construct, a TCR 700 KRAS G12V specific TCR T Cell, or an mCherry control construct. Human T cells were transduced by centrifugation on RetroNectin coated plates with retroviral supernatant from viral packaging cells and then left to rest for 6 days before use. Transduction efficiency was calculated by flow cytometric staining with a murine TCR beta antibody or by analyzing mCherry levels for the control T cells on a Cytoflex S flow cytometer (Beckman Coulter). Monocytes were matured according to manufacturer's instructions (StemCell).

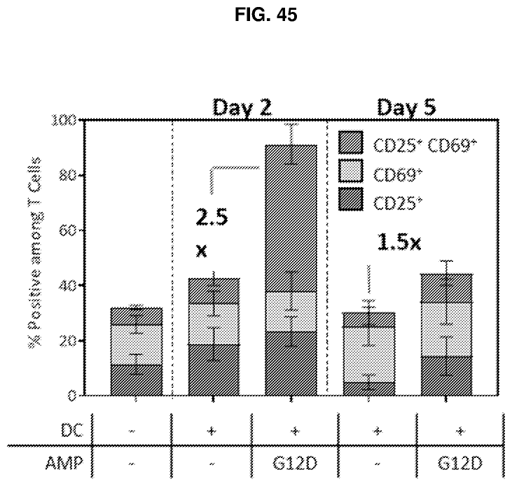

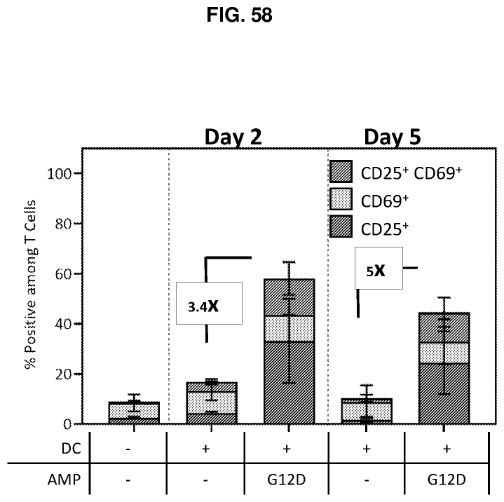

Five days after T cells were rested, mature human dendritic cells were labeled with PBS, soluble, or amp KRAS peptides overnight for 18 hours. The next day, the cells were washed and counted. Human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies (FIG. 45 and FIG. 58). Human TCR T cells were cultured with autologous dendritic cells at a 2:1 T Cell: Dendritic Cell ratio overnight for 18 hours.

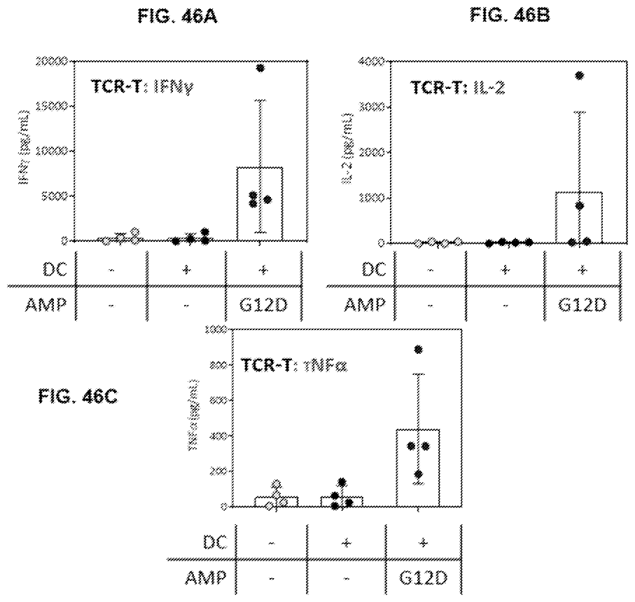

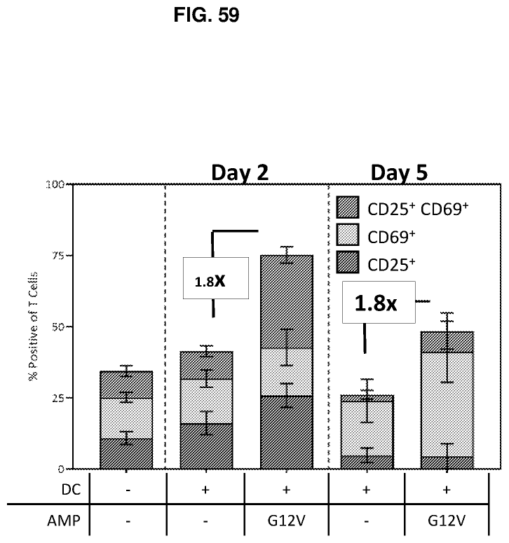

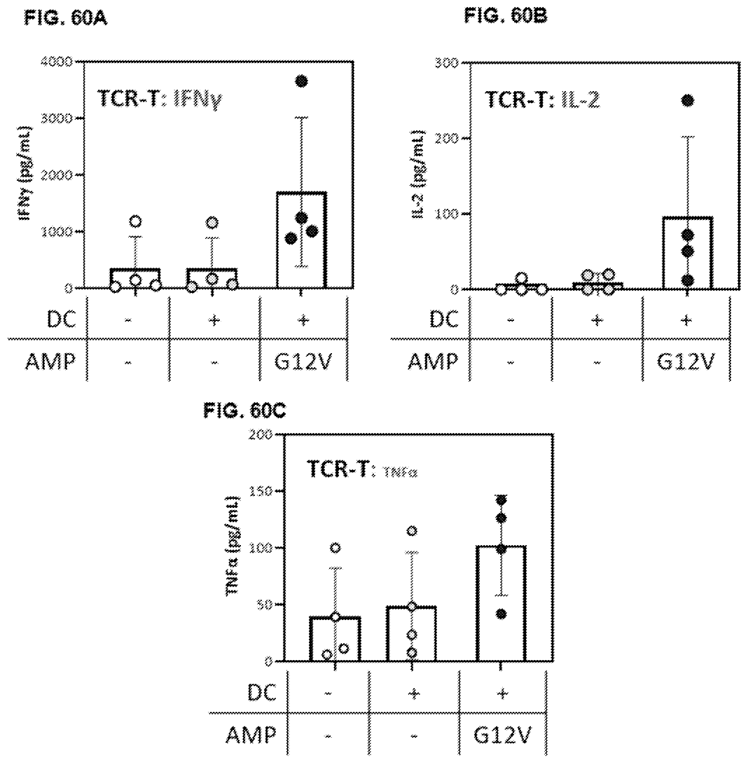

On Day 2 and Day 5 after the dendritic cells were labeled, human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies (FIG. 45 and FIG. 59). The human T cells were also analyzed for the amount of IFNγ, IL-2, and TNFαγ that was present (FIGS. 46A-46C and FIGS. 60A-60C). These data were generated from 4 independent experiments with two different human PBMC donors.

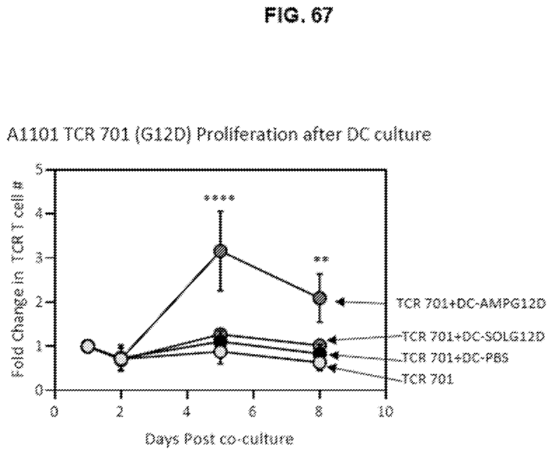

The cell cultures transfected with the TCR 701 KRAS G12D specific TCR T Cell construct, were counted on days 1, 2, 5, and 8 post co-culture (FIG. 67) and were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies to get the percentage of TCR T Cells in the culture. These figures are generated from 3 independent experiments with two different human PBMC donors. These results show that TCR 701 proliferation is specifically enhanced by G12D peptide labeling of autologous dendritic cells (DC).

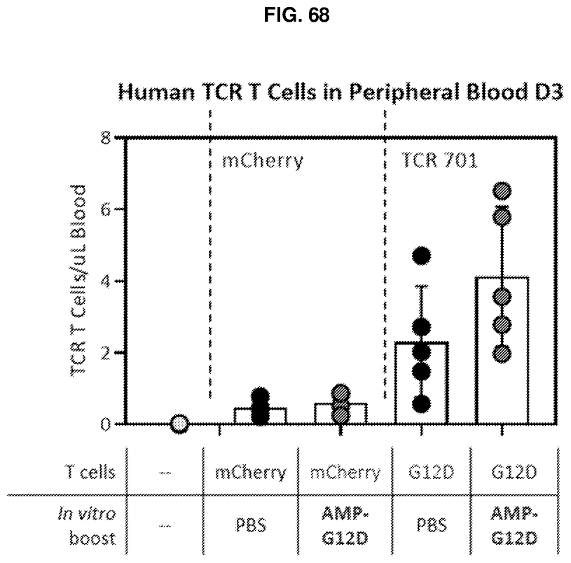

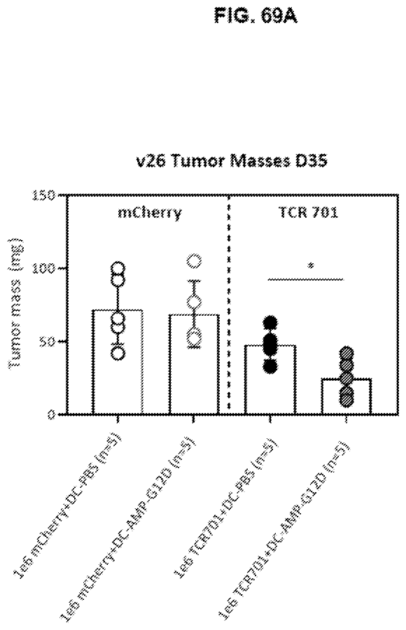

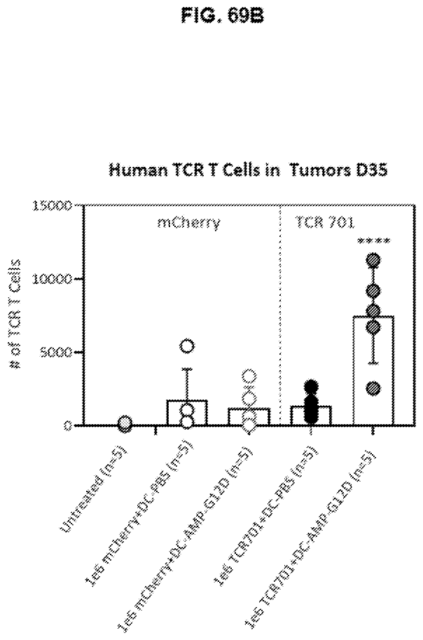

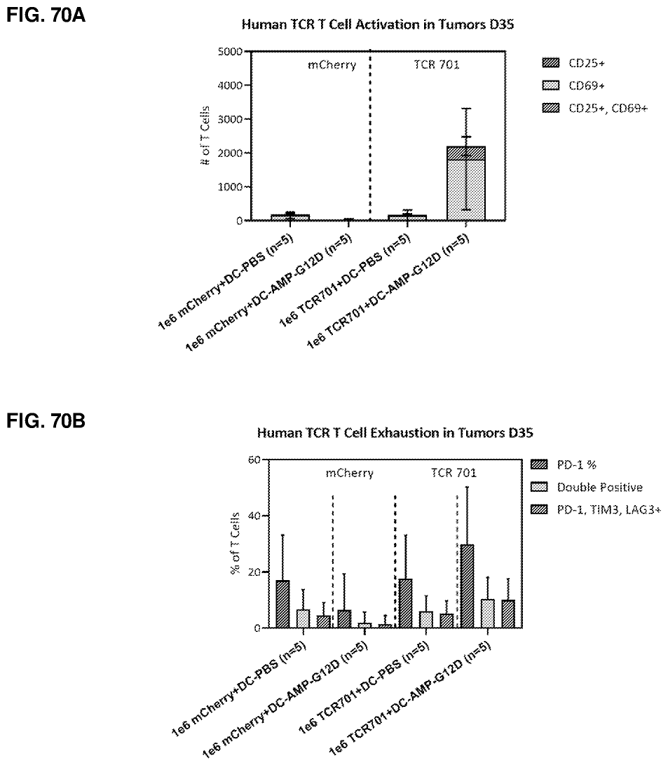

Following co-culture, human T cells were isolated by a negative bead selection, characterized by flow cytometry, counted and infused into 10 day Panc-1 (HLA A11+, KRAS G12D+) tumor bearing NSG mice, which has been previously described, see, e.g., Takakura et al. PLoS One. 2015 Dec. 7; 10(12); Kim et al. Oncol Rep. 2013 September; 30(3):1129-36; and Yu et al. Mol Cancer Ther, 2009 Jan. 1; 8(1). Mice were bled on day 3 to assess T cell engraftment (FIG. 68) and then euthanized on day 35 and tumors analyzed for T cell persistence (FIGS. 69A and 69B). 35 days after infusion of the T cells, the mice were euthanized and tumors were mechanically dissociated and analyzed by flow cytometry on a Cytoflex S flow cytometer (Beckman Coulter) after staining with murine anti-TCR beta, human anti-CD3, anti-CD4, anti-CD8, anti-CD25, anti-CD69, anti-PD-1, anti-TIM-3, and anti-LAG-3 antibodies (FIGS. 70A and 70B). These results show that TCR 701 is specifically activated by G1 2D peptide labeling of autologous dendritic cells (DC) and enhances T cell persistence in the NSG tumor model.

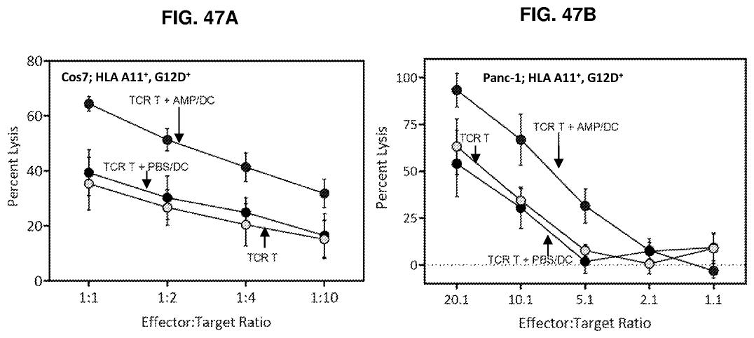

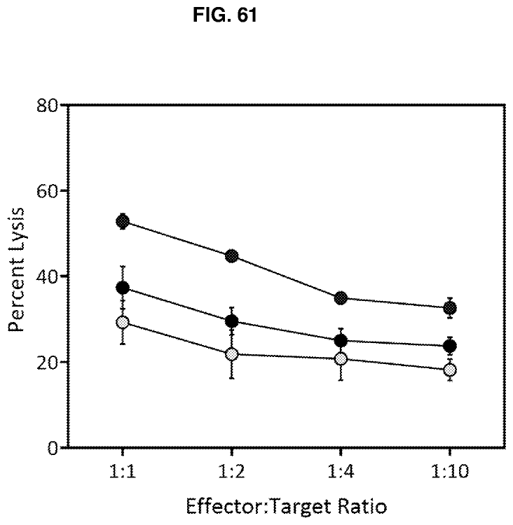

After overnight culture with labeled autologous human dendritic cells, activated HLA A*11:01 TCR T cells were counted and cultured at various Effector to Target Ratios with either Cos-7 target cells expressing luciferase gene, HLA A*11:01, and the KRAS G12D mutation of the KRAS G12D mutation, or a Panc-1 human derived tumor line that also expresses a luciferase gene, HLA A*11:01, and the KRAS G12D mutation KRAS G12D mutation to determine specific lysis (FIGS. 47A, 47B, and 61). 5×10⁴ target cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, One-Glo reagent (Promega) was added to each well. Emitted luminescence of each sample (sample signal) was detected by a Synergy H1 Hybrid plate reader (BioTek). Percent lysis was determined as [1−(sample signal/max signal)]×100. These figures are generated from 4 independent experiments with two different human PBMC donors.

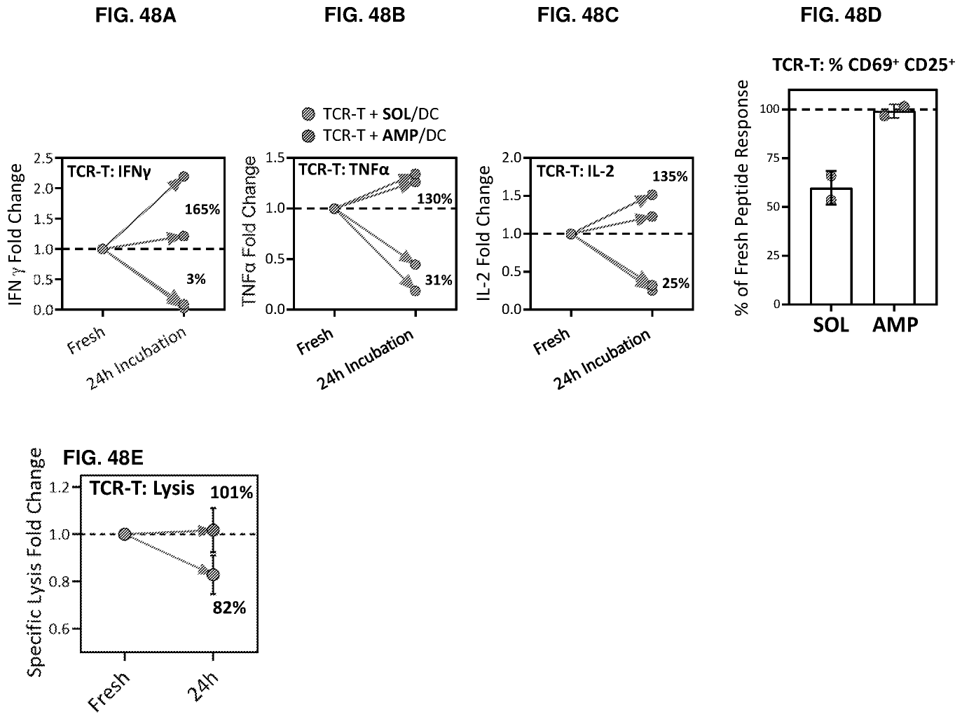

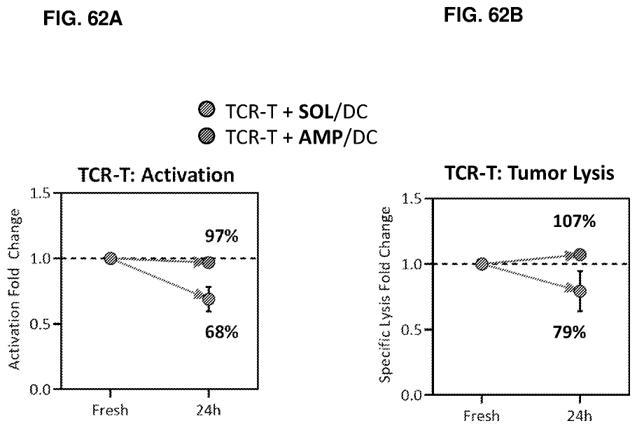

Four days after T cells were rested, Soluble or Amphiphile KRAS peptides were incubated overnight at 37 degrees Celsius in human serum to imitate in vivo conditions. Five days after T cells were rested, mature human dendritic cells were labeled with the overnight incubated soluble or amp KRAS peptides or freshly prepared soluble or AMP kras peptides overnight for 18 hours. The next day, the cells were washed and counted. Human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies (FIG. 48D). Human TCR T cells were cultured with autologous DCs at a 2:1 T Cell: Dendritic Cell ratio overnight for 18 hours. Supernatant fluid was collected from TCR T Cell: Dendritic Cell cultures and analyzed by Luminex for secretion of IL-2 or INFg human cytokines (FIGS. 48A-48C). Additionally, the fold change in activation and tumor lysis were measured (FIGS. 48E, 62A, and 62B). These figures are generated from 2 independent experiments with two different human PBMC donors.

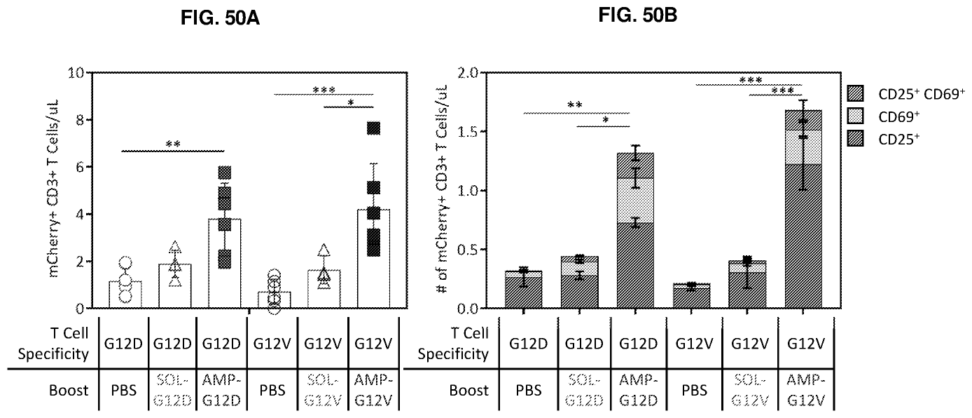

In another experiment, naïve, non-tumor-bearing 6-8 week old C57BL/6 HLA A1101 mice (Taconic) were randomized into different treatment cohorts. On day −1, mice were treated subcutaneously via tail base with PBS, soluble, or amphiphile KRAS peptide vaccine. This was followed by a tail vein intravenous injection of 3×10⁶ T cells previously isolated from splenocytes of 6-8 week old C57BL/6 HLA A1101 mice (Taconic) via a negative T cell isolation kit (StemCell) and retrovirally transduced with either a G12D KRAS specific TCR construct (TCR6) or a G12V KRAS specific TCR construct (TCR3) on day 0. Both constructs also contained mCherry so that transduction efficiency could be calculated and the KRAS specific TCR T cells could be identified in vivo. A subsequent booster dose of vaccine was given on days 3, 7, 10 and 14 via subcutaneous tail base injection. Peripheral blood was collected 15 days after T cell injection to analyze for T cells present in circulating blood by flow cytometry. The number of mCherry+ T cells that were CD3+ were measured (FIG. 50A) as well as the number of mCherry+CD3+ T cells that were CD25+, CD69+, or CD25+ and CD69+(FIG. 50B). On day 15, the data showed that amphiphilic vaccination led to greater number of KRAS specific TCR-T Cells in peripheral blood as determined by the number of mCherry+ T cells. Additionally, they were more activated from the amphiphile vaccination combination.

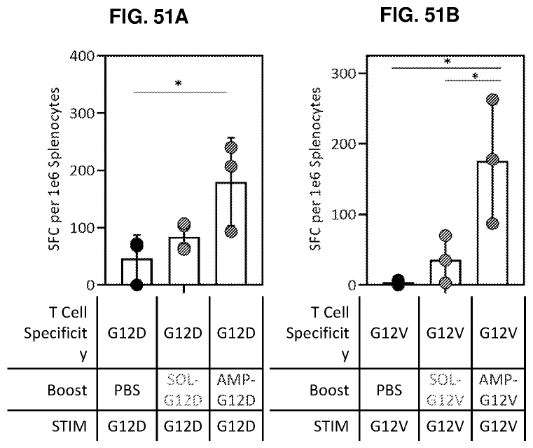

Mice were euthanized on day 15 post adoptive T cell transfer for analysis of spleens, lymph nodes, and lungs. Spleen tissue was processed and stimulated with KRAS mutant peptides in an ELISPOT assay overnight to determine the KRAS Specific T cell response within the spleen of the respective T cell groups (FIGS. 51A and 51B). On day 15, the amphiphile vaccination showed a greater number of KRAS specific TCR-T Cells in spleen as determined by the number of spot forming cells in an ELISPOT assay.

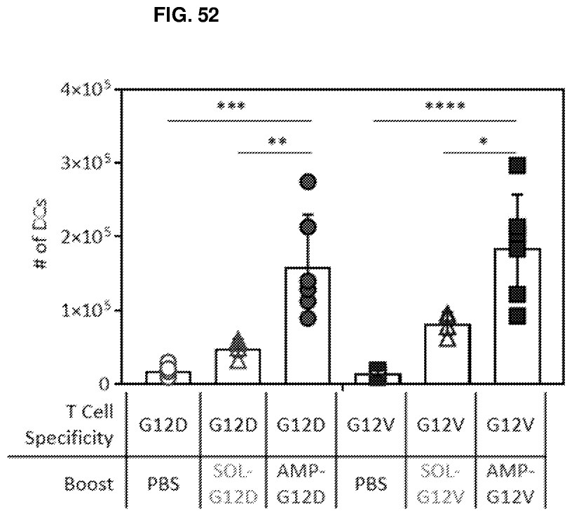

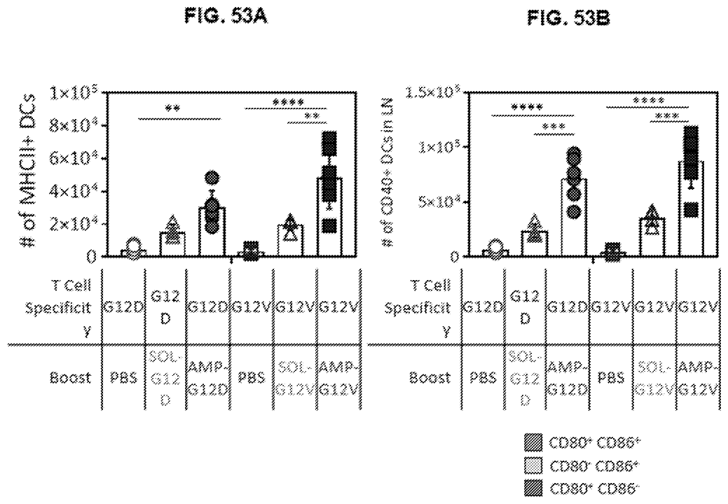

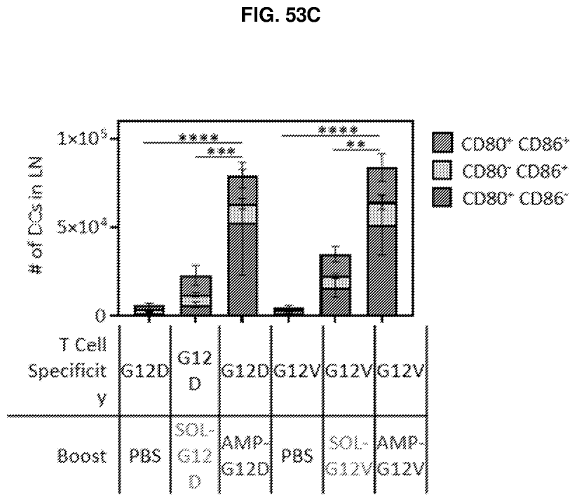

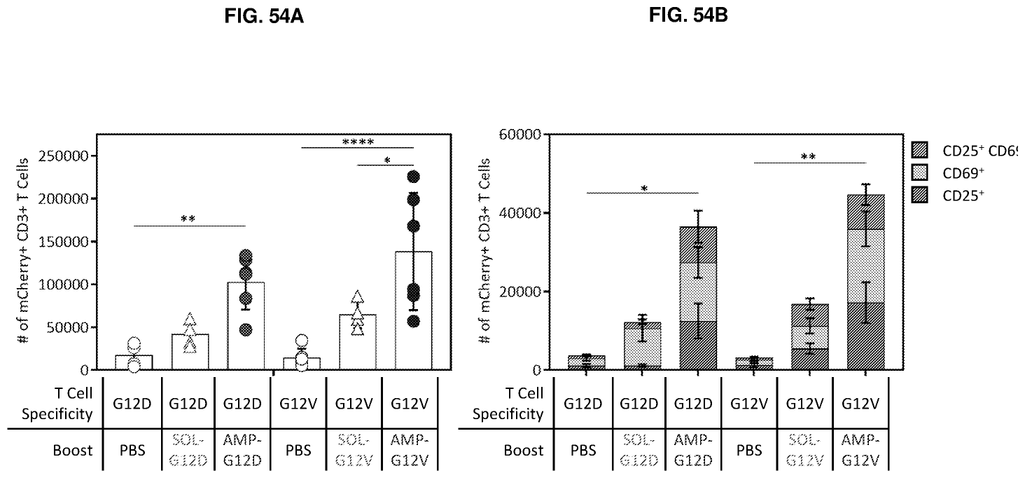

Lymph node tissue was processed and cells were counted followed by flow cytometry analysis to determine the number of dendritic cells present in the lymph nodes (FIG. 52). On day 15, the amphiphilic vaccine led to a greater number of dendritic cells in the lymph nodes. The tissue from the lymph nodes was also analyzed for the number of MHCII+ dendritic cells, CD40+ dendritic cells, and CD80+, CD86+, or CD86+ and CD80+ dendritic cells (FIGS. 53A-53C). On day 15, the amphiphilic vaccine led to a greater number of dendritic cells as well as a greater number of activated dendritic cells in the lymph nodes. Additionally, the lymph node tissue was analyzed for the number of mCherry+ and CD3+ T cells (FIG. 54A), and the number of mCherry+CD3+ T cells that were CD25+, CD69+, or CD25+ and CD69+(FIG. 54B). On day 15, the amphiphile vaccine and subsequent dendritic cell activation and licensing led to a greater number of KRAS specific TCR-T Cells in lymph nodes as determined by the number of mCherry+ T cells in lymph nodes of treated mice.

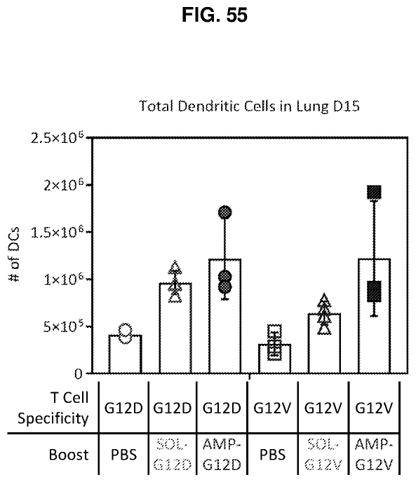

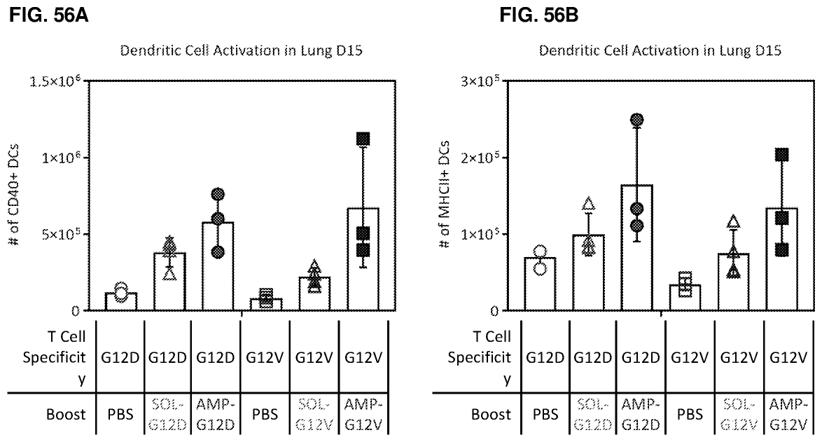

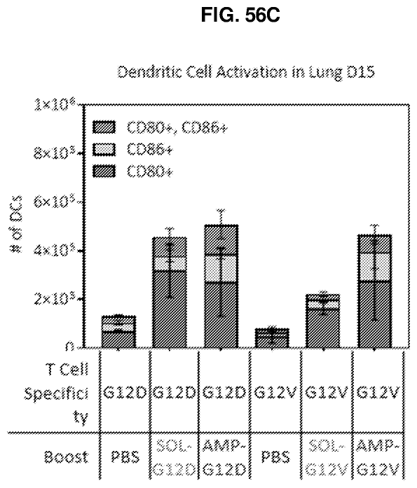

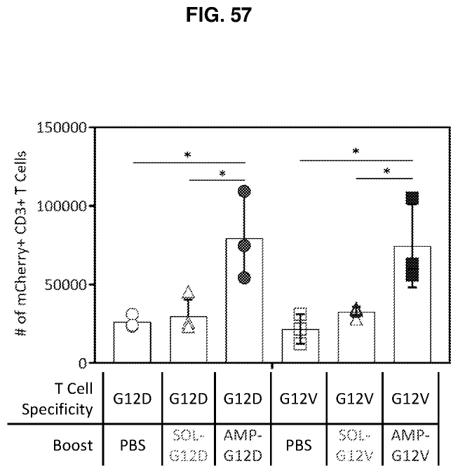

Lung tissue was processed and cells were counted followed by flow cytometry analysis to determine the number of dendritic cells present in the lungs of treated mice (FIG. 55). The tissue from the lungs was also analyzed for the number of MHCII+ dendritic cells, CD40+ dendritic cells, and CD80+, CD86+, or CD86+ and CD80+ dendritic cells (FIGS. 56A-56C). On day 15, the amphiphilic vaccine led to a greater number of dendritic cells as well as a greater number of activated dendritic cells in the lungs. Additionally, the lung tissue was analyzed for the number of mCherry+ and CD3+ T cells (FIG. 57), On day 15, the amphiphile vaccine and subsequent dendritic cell activation and licensing led to a greater number of KRAS specific TCR-T Cells in lungs as determined by the number of mCherry+ T cells in lungs of treated mice.

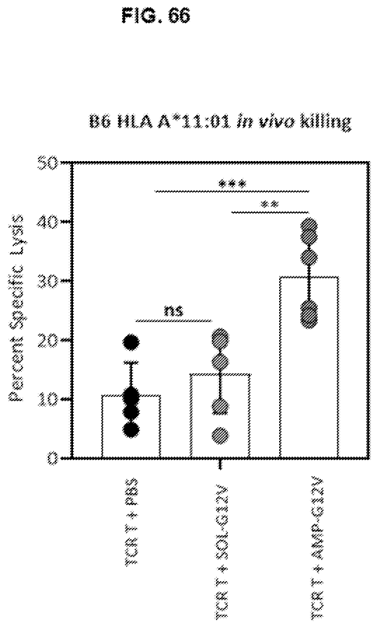

A separate cohort of C57BL/6 HLA A1101 mice were euthanized on day 14 and the splenocytes were harvested. The splenocytes were split into two groups. One group was pulsed with a G12V peptide and one was not. Both groups were then labeled with varying concentrations of fluorescent carboxyfluorescein succinimidyl ester (CFSE), washed 3 time, combined at a 1:1 ratio, and injected into the treatment cohort of mice. Mice were then euthanized on day 15 post adoptive T cell transfer, 1 day after the labeled splenocyte infusion, and the spleens were analyzed for the presence of the CFSE to determine the specific lysis of the KRAS G12V labeled splenocytes in an in vivo killing assay (FIG. 66). Enhanced in vivo killing was observed for splenocytes pulsed with G12V in groups given a TCR-T and AMP-G12V vaccination regimen.

### Example 11. Amphiphilic Boosting of E7 Specific TCR T Cells

Human peripheral blood mononuclear cells were isolated from an HLA A*02:01 donor leukopack (StemExpress). Monocytes and T cells were further isolated by a negative monocyte or T cell isolation (StemCell) kit, respectively. Following negative bead selection, human T cells were subsequently activated with CD3/CD28 Dynabeads at a bead:cell ratio of 1:1 (Invitrogen). Cells were expanded in vitro by culturing in RPMI1640 supplemented with 10% heat-inactivated FBS, sodium pyruvate, 1% penicillin/streptomycin, 2-mercaptoethanol, and 50 IU/mL of recombinant human IL-2. 24 and 48 hours after initial expansion, T cells were spinoculated with viral supernatant collected from Phoenix-Ampho cells transfected with the 1 G4, E7 specific TCR T Cell construct or an mCherry control construct. Human T cells were transduced by centrifugation on RetroNectin coated plates with retroviral supernatant from viral packaging cells and then left to rest for 6 days before use. Transduction efficiency was calculated by flow cytometric staining with a murine TCR beta antibody or by analyzing mCherry levels for the control T cells on a Cytoflex S flow cytometer (Beckman Coulter). Monocytes were matured according to manufacturer's instructions (StemCell).

Five days after T cells were rested, mature human dendritic cells were labeled with PBS, soluble, or amp E7 peptides overnight for 18 hours. The next day, the cells were washed and counted. Human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies. Human TCR T cells were cultured with autologous DCs at a 2:1 T Cell: Dendritic Cell ratio overnight for 18 hours.

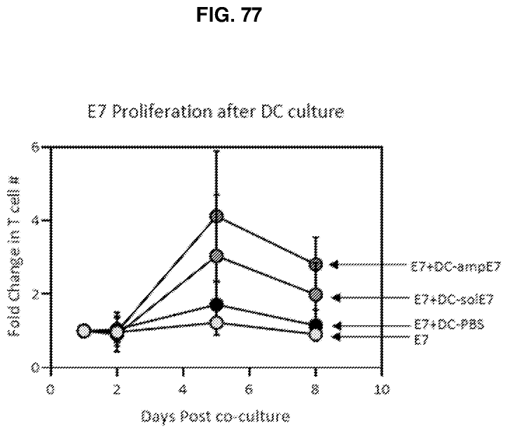

The cell cultures were counted on days 1, 2, 5, and 8 post co-culture (FIG. 77) and were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies to get the percentage of TCR T Cells in the culture. E7 specific TCT-T cells are specifically activated by amp-E7 peptide labeling of autologous dendritic cells to enhance specific tumor lysis.

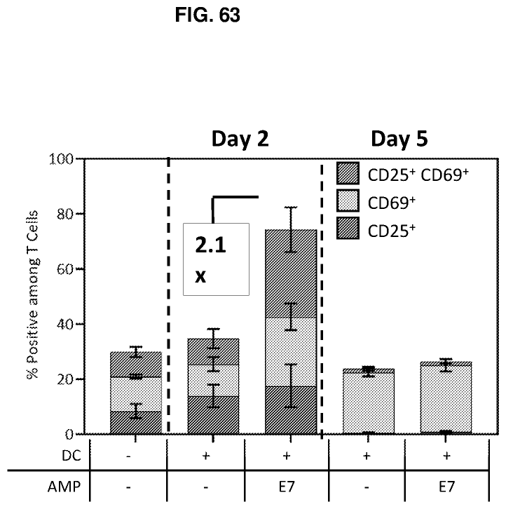

On Day 2 and D5 after the DCs were labeled, human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies (FIG. 63). These figures are generated from 4 independent experiments with two different human PBMC donors.

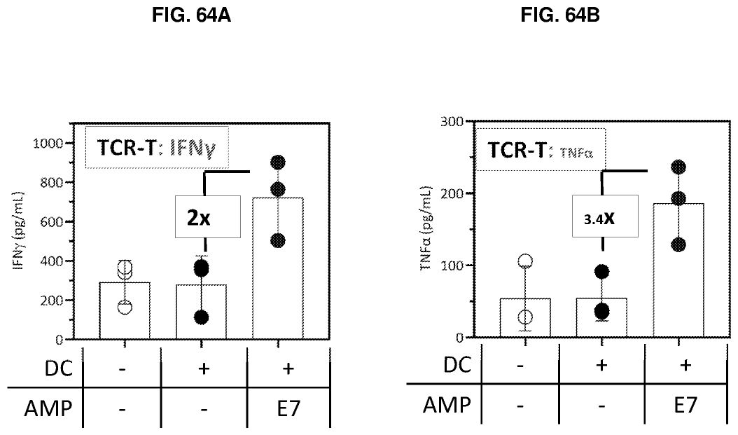

On Day 2 after the DCs were labeled, Supernatant liquid was collected from TCR T Cell: Dendritic Cell cultures and analyzed by Luminex for secretion of IL-2, TNFα or INFγ human cytokines (FIGS. 64A and 64B). These figures are generated from 4 independent experiments with two different human PBMC donors.

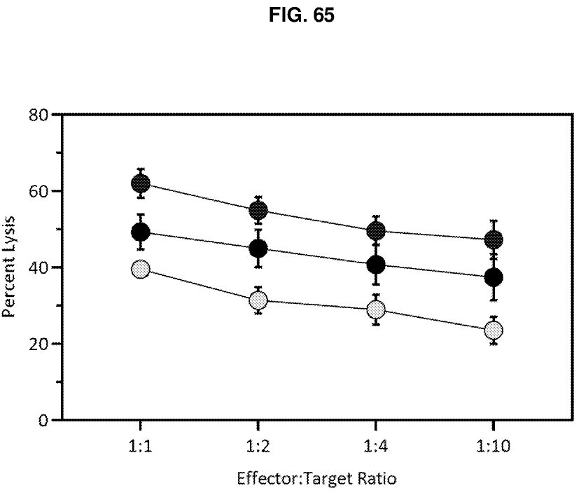

After overnight culture with labeled autologous human DCs, activated HLA A*02:01 TCR T cells were counted and cultured at various Effector to Target Ratios with a Ca Ski human derived tumor line that also expresses a luciferase gene, HLA A*02:01, and the HPV16 E7 epitope to determine specific lysis (FIG. 65). 5×10⁴ target cells expressing firefly luciferase were cocultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, One-Glo reagent (Promega) was added to each well. Emitted luminescence of each sample (sample signal) was detected by a Synergy H1 Hybrid plate reader (BioTek). Percent lysis was determined as [1−(sample signal/max signal)]×100. These figures are generated from 4 independent experiments with three different human PBMC donors.

### Example 12. Amphiphile Boosting of NY-ESO-1 Specific TCR T Cells

Human peripheral blood mononuclear cells were isolated from an HLA A*02:01 donor leukopack (StemExpress). Monocytes and T cells were further isolated by a negative monocyte or T cell isolation (StemCell) kit, respectively. Following negative bead selection, human T cells were subsequently activated with CD3/CD28 Dynabeads at a bead:cell ratio of 1:1 (Invitrogen). Cells were expanded in vitro by culturing in RPMI1640 supplemented with 10% heat-inactivated FBS, sodium pyruvate, 1% penicillin/streptomycin, 2-mercaptoethanol, and 50 IU/mL of recombinant human IL-2. 24 and 48 hours after initial expansion, T cells were spinoculated with viral supernatant collected from Phoenix-Ampho cells transfected with the 1 G4 TCR, NY-ESO-1 specific TCR T Cell construct or an mCherry or MART-1 targeted DMF5 TCR T control construct. Human T cells were transduced by centrifugation on RetroNectin coated plates with retroviral supernatant from viral packaging cells and then left to rest for 6 days before use. Transduction efficiency was calculated by flow cytometric staining with a murine TCR beta antibody or by analyzing mCherry levels on a Cytoflex S flow cytometer (Beckman Coulter). Monocytes were matured according to manufacturer's instructions (StemCell).

Five days after T cells were rested, mature human dendritic cells were labeled with PBS, soluble, or amp NY-ESO-1 peptides overnight for 18 hours. The next day, the cells were washed and counted. Human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies. Human TCR T cells were cultured with autologous DCs at a 2:1 T Cell: Dendritic Cell ratio overnight for 18 hours.

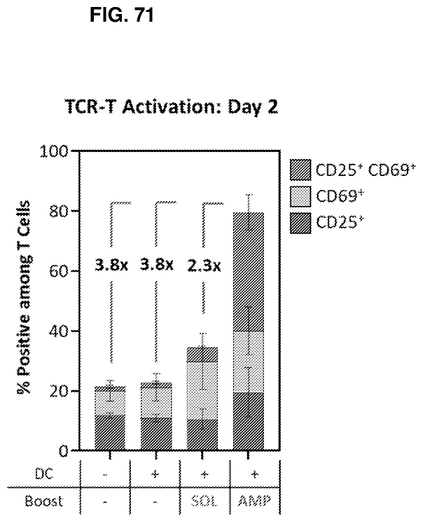

On Day 2 after the dendritic cells were labeled, human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies (FIG. 71). These figures are generated from 2 independent experiments with two different human PBMC donors. These results support that 1 G4 specifically activated NY-ESO-1 AMP-peptide labeling of autologous dendritic cells.

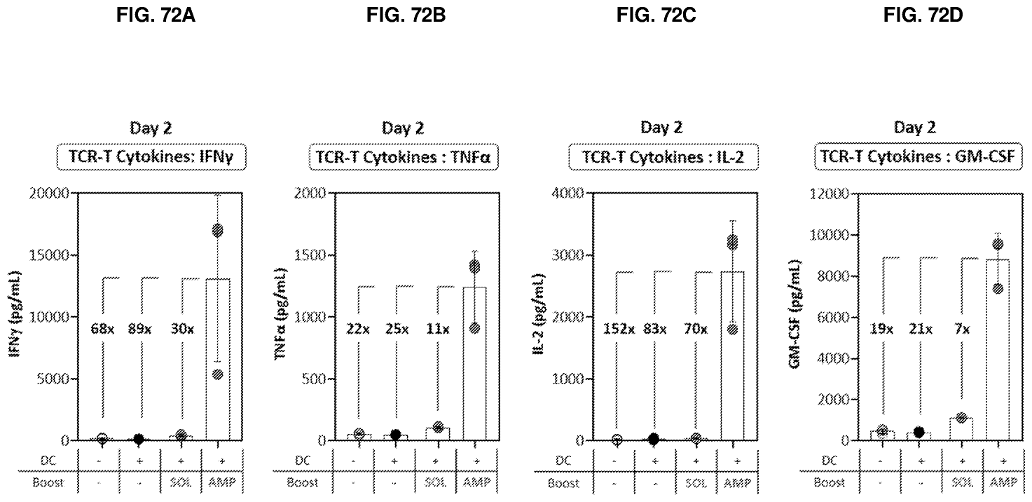

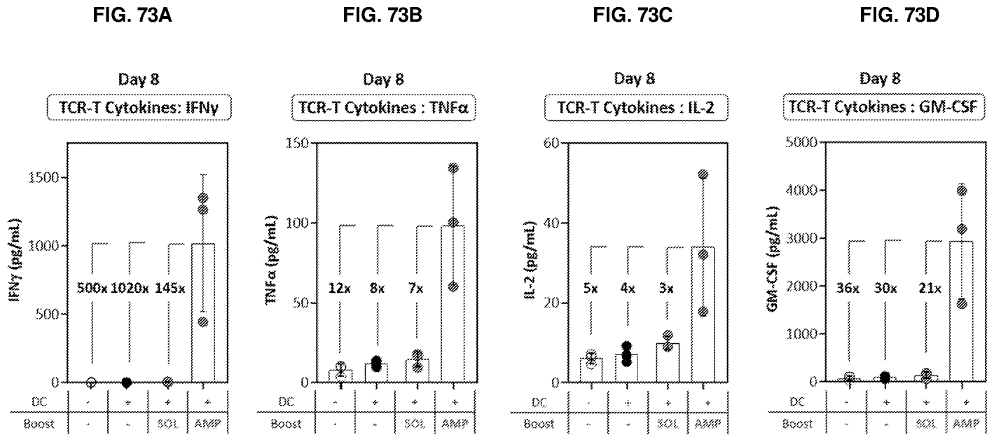

On Days 2 and 8 after the dendritic cells were labeled, supernatant liquid was collected from TCR T Cell: Dendritic Cell cultures and analyzed by Luminex for secretion of IL-2, TNFα, GM-CSF or INFγ human cytokines (FIGS. 72A-72D and FIGS. 73A-73D). These results support that 1 G4 is specifically activated by NY-ESO-1 AMP-peptide labeling of autologous dendritic cells.

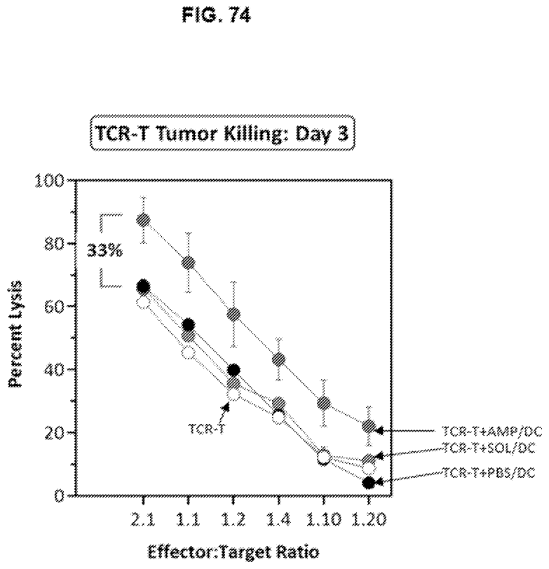

After overnight culture with labeled autologous human dendritic cells, activated HLA A*02:01 NY-ESO-1 specific 1 G4 TCR T cells were counted and cultured at various Effector to Target Ratios with an A375 human derived tumor line that also expresses a luciferase gene, HLA A*02:01, and the NY-ESO-1 tumor associated antigen to determine specific lysis. 5×10⁴ target cells expressing firefly luciferase were co-cultured with adoptively transferred T cells at various effector-to-target ratios in triplicates in black-walled 96-well plates in a total volume of 200 μL of cell media. Target cells alone were plated at the same cell density to determine the maximal luciferase expression as a reference (max signal). 24 hours later, One-Glo reagent (Promega) was added to each well. E mitted luminescence of each sample (sample signal) was detected by a Synergy H1 Hybrid plate reader (BioTek). Percent lysis was determined as [1−(sample signal/max signal)]×100 on day 3 (FIG. 74). These results support that 1 G4 is specifically activated by NY-ESO-1 AMP-peptide labeling of autologous dendritic cells.

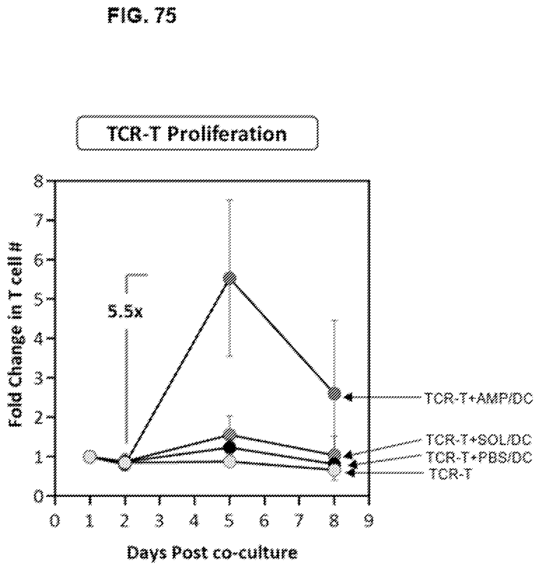

The cell cultures were counted on days 1, 2, 5, and 8 post co-culture (FIG. 75) and were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies to get the percentage of TCR T Cells in the culture. These results show that 1 G4 TCR T cell proliferation is specifically enhanced by AMP-NY-ESO-1 peptide labeling of autologous dendritic cells.

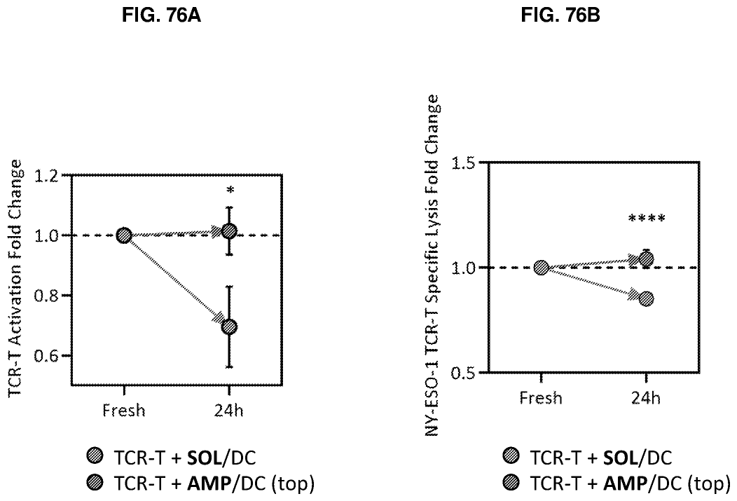

Four days after T cells were rested, soluble or amphiphile NY-ESO-1 peptides were incubated overnight at 37 degrees Celsius in human serum to imitate in vivo conditions. Five days after T cells were rested, mature human dendritic cells were labeled with the overnight incubated soluble or AMP NY-ESO-1 peptides or freshly prepared soluble or AMP NY-ESO-1 peptides overnight for 18 hours. The next day, the cells were washed and counted. Human T cells were characterized by flow cytometry on a Cytoflex S flow cytometer (Beckman Coulter) after staining with murine anti-TCR beta, human anti-CD3, anti-CD4, anti-CD8, anti-CD25, and anti-CD69 antibodies. Human TCR T cells were cultured with autologous dendritic cells at a 2:1 T Cell: Dendritic Cell ratio overnight for 18 hours. Activation was assayed by CD25 and CD69 (FIG. 76A). Expression and tumor lysis was determined by overnight 24 hour cytotoxicity assays as previously described (FIG. 76B). These results support that AMP peptides maintain stability and boost NY-ESO-1 specific TCT T cells in mock in vivo conditions. These figures were generated from 2 independent experiments with two different human PBMC donors.

### NUMBERED EMBODIMENTS

1. A method of stimulating an immune response to a target cell population or target tissue in a subject, the method comprising administering to the subject (1) an amphiphilic ligand conjugate comprising a lipid, a peptide, and, optionally, a linker, and (2) a T-cell receptor (TCR) modified immune cell, wherein the TCR binds the peptide of the amphiphilic ligand conjugate.

2. A method of stimulating an immune response to a target cell population or target tissue in a subject, the method comprising administering to the subject (1) an amphiphilic ligand conjugate comprising a lipid, a ligand for a mucosal-associated invariant T-cell (MAIT), and, optionally, a linker, and (2) a T-cell receptor (TCR) modified immune cell, wherein the TCR binds the ligand of the amphiphilic ligand conjugate.

3. The method of embodiment 1 or 2, further comprising administering an adjuvant to the subject.

4. The method of any one of embodiments 1-3, wherein the lipid of the amphiphilic ligand conjugate inserts into a cell membrane under physiological conditions, binds albumin under physiological conditions, or both.

5. The method of any one of embodiments 1-4, wherein the lipid of the amphiphilic ligand conjugate is a diacyl lipid.

6. The method of embodiment 5, wherein the diacyl lipid of the amphiphilic ligand conjugate comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.

7. The method of embodiment 6, wherein the lipid is 1,2-distearoyl-sn-glycero-3-phosphoethanolamine (DSPE).

8. The method of any one of embodiments 1-7, wherein the linker is selected from the group consisting of a hydrophilic polymer, a string of hydrophilic amino acids, a polysaccharide, and an oligonucleotide, or a combination thereof.

9. The method of embodiment 8, wherein the linker comprises “N” polyethylene glycol units, wherein N is between 24-50.

10. The method of embodiment 9, wherein the linker comprises PEG24-amido-PEG24.

11. The method of any one of embodiments 1 and 3-10, wherein the peptide is an antigen, or a fragment thereof.

12. The method of embodiment 11, wherein the antigen, or fragment thereof, is a tumor-associated antigen, or a fragment thereof.

13. The method of embodiment 11 or embodiment 12, wherein the antigen, or fragment thereof, comprises between 3 amino acids and 50 amino acids.

14. The method of embodiment 13, wherein the antigen comprises a fragment of the sequence of any one of SEQ ID NOs: 1-97 or 1125-1183, or comprises Ganglioside G2 or Ganglioside G3.

15. The method of any one of embodiments 1-14, wherein the peptide comprises an amino acid sequence of any one of SEQ ID NOs: 98-1123.

16. The method of embodiment 2, wherein the ligand for a MAIT cell is a small molecule metabolite ligand.

17. The method of embodiment 2, wherein the ligand for a MAIT cell is a valine-citrulline-p-aminobenzyl carbamate modified ligand.

18. The method of embodiment 17, wherein the valine-citrulline-p-aminobenzyl carbamate modified ligand is a valine-citrulline-p-aminobenzyl carbamate modified 5-amino-6-D-ribityl prodrug.

19. The method of embodiment 2, wherein the ligand for a MAIT cell is a riboflavin metabolite or a drug metabolite.

20. The method of embodiment 19, wherein the riboflavin metabolite is 5-(2-oxopropylideneamino)-6-d-ribitylaminouracil, 5-(2-oxoethylideneamino)-6-D-ribitylaminouracil, 6,7-dimethyl-8-D-ribityllumazine, 7-hydroxy-6-methyl-8-D-ribityllumazine, 6-hydroxymethyl-8-D-ribityl-lumazine, 6-(1H-indol-3-yl)-7-hydroxy-8-ribityllumazine, or 6-(2-carboxyethyl)-7-hydroxy8-ribityllumazine.

21. The method of embodiment 19, wherein the drug metabolite is benzbromarone, chloroxine, diclofenac, 5-hydroxy diclofenac, 4-hydroxy diclofenac, floxuridine, galangin, menadione sodium bisulfate, mercaptopurine, or tetrahydroxy-1,4-quinone hydrate.

22. The method of any one of embodiments 1-21, wherein the amphiphilic ligand conjugate is trafficked to a lymph node.

23. The method of embodiment 22 wherein the amphiphilic ligand conjugate is trafficked to an inguinal lymph node or an axillary lymph node.

24. The method of embodiment 22 or embodiment 23, wherein the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

25. The method of any one of embodiments 1-24, wherein the immune cell is a T cell, a B cell, a natural killer (NK) cell, a macrophage, a neutrophil, a dendritic cell, a mast cell, an eosinophil, or a basophil.

26. The method of embodiment 25, wherein the immune cell is a T cell.

27. The method of embodiment 2, wherein the immune cell is a human mucosal-associated T cell.

28. The method of any one of embodiments 1-27, wherein the immune response is an anti-tumor immune response.

29. The method of any one of embodiments 1-28, wherein the target cell population or the target tissue is a tumor cell population or a tumor tissue.

30. The method of any one of embodiments 1-29, wherein the method comprises reducing or decreasing the size of the tumor tissue or inhibiting growth of the tumor cell population or the tumor tissue in the subject.

31. The method of any one of embodiments 1-30, wherein the method comprises activating the immune cell, expanding the immune cell, and/or increasing proliferation of the immune cell, wherein the activating, expanding, and/or increasing proliferation is performed ex vivo or in vivo.

32. The method of any one of embodiments 1-31, wherein the subject has a disease, a disorder, or a condition associated with expression or elevated expression of the antigen.

33. The method of any one of embodiment 1-32, wherein the subject is lymphodepleted prior to the administration of the amphiphilic ligand conjugate and TCR modified immune cell.

34. The method of embodiment 33, wherein the lymphodepletion is by sublethal irradiation.

35. The method of any one of embodiments 1-34, wherein the subject is administered the amphiphilic ligand conjugate prior to receiving the immune cell comprising the TCR.

36. The method of any one of embodiments 1-34, wherein the subject is administered the amphiphilic ligand conjugate after receiving the immune cell comprising the TCR.

37. The method of any one of embodiments 1-34, wherein the amphiphilic ligand conjugate and the immune cell comprising the TCR are administered simultaneously.

38. The method of any one of embodiments 1-37, wherein the amphiphilic ligand conjugate and/or the TCR modified immune cell are administered in a composition comprising a pharmaceutically acceptable carrier.

39. The method of embodiment 38, wherein the composition further comprises an adjuvant.

40. The method of embodiment 3 or 39, wherein the adjuvant is an amphiphilic oligonucleotide conjugate comprising an immunostimulatory oligonucleotide conjugated to a lipid, with or without a linker.

41. A method of activating, proliferating, phenotypically maturing, or inducing acquisition of cytotoxic function of a TCR modified T-cell in vitro, comprising culturing the TCR modified T-cell in the presence of a dendritic cell comprising an amphiphilic ligand conjugate comprising a lipid, a peptide, and, optionally, a linker.

42. A method of activating, proliferating, phenotypically maturing, or inducing acquisition of cytotoxic function of a TCR modified T-cell in vitro, comprising culturing the TCR modified T-cell in the presence of a dendritic cell comprising an amphiphilic ligand conjugate comprising a lipid, a small metabolite ligand, and, optionally, a linker.

43. The method of embodiment 41 or embodiment 42, wherein the lipid of the amphiphilic ligand conjugate is a diacyl lipid.

44. Use of (1) an amphiphilic ligand conjugate comprising a lipid, a peptide, and, optionally, a linker, and (2) a T-cell receptor (TCR) modified immune cell in a method of stimulating an immune response to a target cell population or target tissue in a subject, the method comprising administering to the subject (1) an amphiphilic ligand conjugate comprising a lipid, a peptide, and, optionally, a linker, and (2) a T-cell receptor (TCR) modified immune cell, wherein the TCR binds the peptide of the amphiphilic ligand conjugate.

45. Use of (1) an amphiphilic ligand conjugate comprising a lipid, a ligand for a mucosal-associated invariant T-cell (MAIT), and, optionally, a linker, and (2) a T-cell receptor (TCR) modified immune cell in a method of stimulating an immune response to a target cell population or target tissue in a subject, the method comprising administering to the subject (1) an amphiphilic ligand conjugate comprising a lipid, a ligand for a mucosal-associated invariant T-cell (MAIT), and, optionally, a linker, and (2) a T-cell receptor (TCR) modified immune cell, wherein the TCR binds the ligand of the amphiphilic ligand conjugate.

46. The method of embodiment 11, wherein the fragment is an immunogenic fragment.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of stimulating an immune response to a target cell population or target tissue in a subject, the method comprising administering to the subject (1) an amphiphilic ligand conjugate comprising a lipid, a peptide, and, optionally, a linker, and (2) a T-cell receptor (TCR) modified immune cell, wherein the TCR binds the peptide of the amphiphilic ligand conjugate.

2. A method of stimulating an immune response to a target cell population or target tissue in a subject, the method comprising administering to the subject (1) an amphiphilic ligand conjugate comprising a lipid, a ligand for a mucosal-associated invariant T-cell (MAIT), and, optionally, a linker, and (2) a T-cell receptor (TCR) modified immune cell, wherein the TCR binds the ligand of the amphiphilic ligand conjugate.

3. The method of claim 1 or 2, further comprising administering an adjuvant to the subject.

4. The method of claim 1 or 2, wherein the lipid of the amphiphilic ligand conjugate inserts into a cell membrane under physiological conditions, binds albumin under physiological conditions, or both.

5. The method of claim 1 or 2, wherein the lipid of the amphiphilic ligand conjugate is a diacyl lipid.

6. The method of claim 5, wherein the diacyl lipid of the amphiphilic ligand conjugate comprises acyl chains comprising 12-30 hydrocarbon units, 14-25 hydrocarbon units, 16-20 hydrocarbon units, or 12, 13, 14, 15, 16, 17, 18, 19, 20, 21, 22, 23, 24, 25, 26, 27, 28, 29, or 30 hydrocarbon units.

7. The method of claim 6, wherein the lipid is 1,2-distearoyl-sn-glycero-3-phosphoethanolamine (DSPE).

8. The method of claim 1 or 2, wherein the linker is selected from the group consisting of a hydrophilic polymer, a string of hydrophilic amino acids, a polysaccharide, and an oligonucleotide, or a combination thereof.

9. The method of claim 8, wherein the linker comprises “N” polyethylene glycol units, wherein N is between 24-50.

10. The method of claim 9, wherein the linker comprises PEG24-amido-PEG24.

11. The method of claim 1 or 2, wherein the peptide is an antigen, or a fragment thereof.

12. The method of claim 11, wherein the antigen, or fragment thereof, is a tumor-associated antigen, or a fragment thereof.

13. The method of claim 11, wherein the antigen, or fragment thereof, comprises between 3 amino acids and 50 amino acids.

14. The method of claim 13, wherein the antigen comprises a fragment of the sequence of any one of SEQ ID NOs: 1-97 or 1125-1183, or comprises Ganglioside G2 or Ganglioside G3.

15. The method of claim 1, wherein the peptide comprises an amino acid sequence of any one of SEQ ID NOs: 98-1123.

16. The method of claim 2, wherein the ligand for a MAIT cell is a small molecule metabolite ligand.

17. The method of claim 2, wherein the ligand for a MAIT cell is a valine-citrulline-p-aminobenzyl carbamate modified ligand.

18. The method of claim 17, wherein the valine-citrulline-p-aminobenzyl carbamate modified ligand is a valine-citrulline-p-aminobenzyl carbamate modified 5-amino-6-D-ribityl prodrug.

19. The method of claim 2, wherein the ligand for a MAIT cell is a riboflavin metabolite or a drug metabolite.

20. The method of claim 19, wherein the riboflavin metabolite is 5-(2-oxopropylideneamino)-6-d-ribitylaminouracil, 5-(2-oxoethylideneamino)-6-D-ribitylaminouracil, 6,7-dimethyl-8-D-ribityllumazine, 7-hydroxy-6-methyl-8-D-ribityllumazine, 6-hydroxymethyl-8-D-ribityl-lumazine, 6-(1H-indol-3-yl)-7-hydroxy-8-ribityllumazine, or 6-(2-carboxyethyl)-7-hydroxy8-ribityllumazine.

21. The method of claim 19, wherein the drug metabolite is benzbromarone, chloroxine, diclofenac, 5-hydroxy diclofenac, 4-hydroxy diclofenac, floxuridine, galangin, menadione sodium bisulfate, mercaptopurine, or tetrahydroxy-1,4-quinone hydrate.

22. The method of claim 1 or 2, wherein the amphiphilic ligand conjugate is trafficked to a lymph node.

23. The method of claim 22 wherein the amphiphilic ligand conjugate is trafficked to an inguinal lymph node or an axillary lymph node.

24. The method of claim 22, wherein the amphiphilic ligand conjugate is retained in the lymph node for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, or at least 25 days.

25. The method of claim 1 or 2, wherein the immune cell is a T cell, a B cell, a natural killer (NK) cell, a macrophage, a neutrophil, a dendritic cell, a mast cell, an eosinophil, or a basophil.

26. The method of claim 25, wherein the immune cell is a T cell.

27. The method of claim 2, wherein the immune cell is a human mucosal-associated T cell.

28. The method of claim 1, wherein the immune response is an anti-tumor immune response.

29. The method of claim 1 or 2, wherein the target cell population or the target tissue is a tumor cell population or a tumor tissue.

30. The method of claim 1 or 2, wherein the method comprises reducing or decreasing the size of the tumor tissue or inhibiting growth of the tumor cell population or the tumor tissue in the subject.

31. The method of claim 1 or 2, wherein the method comprises activating the immune cell, expanding the immune cell, and/or increasing proliferation of the immune cell.

32. The method of claim 1 or 2, wherein the subject has a disease, a disorder, or a condition associated with expression or elevated expression of the antigen.

33. The method of claim 1 or 2, wherein the subject is lymphodepleted prior to the administration of the amphiphilic ligand conjugate and TCR modified immune cell.

34. The method of claim 33, wherein the lymphodepletion is by sublethal irradiation.

35. The method of claim 1 or 2, wherein the subject is administered the amphiphilic ligand conjugate prior to receiving the immune cell comprising the TCR.

36. The method of claim 1 or 2, wherein the subject is administered the amphiphilic ligand conjugate after receiving the immune cell comprising the TCR.

37. The method of claim 1 or 2, wherein the amphiphilic ligand conjugate and the immune cell comprising the TCR are administered simultaneously.

38. The method of claim 1 or 2, wherein the amphiphilic ligand conjugate and/or the TCR modified immune cell are administered in a composition comprising a pharmaceutically acceptable carrier.

39. The method of claim 38, wherein the composition further comprises an adjuvant.

40. The method of claim 3, wherein the adjuvant is an amphiphilic oligonucleotide conjugate comprising an immunostimulatory oligonucleotide conjugated to a lipid, with or without a linker.

41. A method of activating, proliferating, phenotypically maturing, or inducing acquisition of cytotoxic function of a TCR modified T-cell in vitro, comprising culturing the TCR modified T-cell in the presence of a dendritic cell comprising an amphiphilic ligand conjugate comprising a lipid, a peptide, and, optionally, a linker.

42. A method of activating, proliferating, phenotypically maturing, or inducing acquisition of cytotoxic function of a TCR modified T-cell in vitro, comprising culturing the TCR modified T-cell in the presence of a dendritic cell comprising an amphiphilic ligand conjugate comprising a lipid, a small metabolite ligand, and, optionally, a linker.

43. The method of claim 41, wherein the lipid of the amphiphilic ligand conjugate is a diacyl lipid.

44. The method of claim 42, wherein the lipid of the amphiphilic ligand conjugate is a diacyl lipid.
